SEQUENCE LISTING (76 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 5128 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 4 - DNA, 2331 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaaggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagc

SEQ ID NO: 5 - protein, 712 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDS

SEQ ID NO: 6 - DNA, 4182 bp, Homo sapiens
atttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 7 - protein, 638 aa, Homo sapiens
ISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 8 - DNA, 3206 bp, Mus sp.
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 9 - protein, 892 aa, Mus sp.
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 10 - DNA, 3149 bp, Drosophila melanogaster
catttgtatccgcttggccacgagctttggctgcacttggcaaacttaataaattaaacattgaatcctgcctattgcaacgataatataatctgatttagtgcattaagaacgacaagtagcgattataatagtagattttagcatttgagctaaatttatttcccaaccgcgtcttgggattgcgtatgcgtgagccagtacctgcatgtgtgtgtgttttggaatgtggccctgcacgaaattcaaatagtgaccatccttgagattttgcatactggcaagatggacgagcacacgttaaacgacctgttggagtgctccgtgtgtcttgagcgactggacaccacatcgaaggtgctgccatgccagcacaccttctgccgcaaatgcttgcaggacattgtggccagtcagcacaagttgcgatgcccggagtgccgcatcctggtctcttgcaaaattgatgagctgcctccaaacgtcttgctgatgcgaatcttagaaggcatgaaacaaaatgcagcagctggcaaaggagaagaaaagggagaggagactgaaacacagccggaaagggccaaacctcagccgccagcggaatcagtggccccgcctgacaaccaactactccagctgcagtcacatcagcaatctcatcagccggctcgtcacaagcaacgtcgatttctactcccccacgcctatgccctctttgacttcgcctccggtgaagccaccgatctaaagttcaagaaaggggatctgatactgatcaagcatcgcatcgacaacaactggtttgtgggtcaagcgaatggtcaggagggcacatttcccatcaactacgtcaaggtatcggttccgctgcccatgccgcagtgcattgccatgtatgactttaagatggggcccaacgacgaggagggatgcctcgaatttaagaaaagcactgtaatacaggtaatgcgccgagttgatcataattgggcagaaggacgaattggccagaccatcggaatctttccaatagcattcgttgagctgaatgcagcggccaaaaagctgttggacagcgggctacacacccatccattctgccatccaccgaagcaacaggggcagcgggcccttcctccggttccagttattgatcccacggtggtcacggaatccagttcgggatcctccaattccacgccgggcagcagcaattcaagctccacatccagctcgaataactgcagtccgaatcaccaaatctcactgccgaataccccccaacatgtagtagcttccggatcggcgtctgttcgtttccgtgacaagggagcaaaggagaaacgccactcactaaatgctttgctgggaggaggagctccattaagtctgctgcagaccaaccgccattcggctgaaattcttagcctgccccatgaactaagccgcttggaagtttccagctcaacagctctaaaacccacgtcagccccacagacatcgcgtgtacttaagaccactgttcagcagcagatgcaaccgaatttaccctggggatacttagccctgttcccatacaaaccacgccaaacggatgagctggaattaaaaaagggttgtgtttacattgtgaccgaacgatgtgtggacggttggttcaagggaaaaaactggttggacatcactggagtgttcccgggcaactacctgacgcccctgcgcgcccgcgaccagcagcagttaatgcatcaatggaaatatgttccccaaaatgcagacgcccagatggcacaagtacagcagcatccagttgcaccagatgtgcgactcaacaacatgctgtccatgcaaccgcctgatttgccacctcgtcagcagcaggctaccgccacgaccaccagttgctctgtgtggtcgaaaccagtggaggcgctgttcagcagaaaatcggagcccaagcctgaaactgccacagcttcgactacgagcagcagttcctctggagcagtgggacttatgaggagattaactcacatgaaaacacgctccaaatctccgggagcgtccttgcagcaagttccgaaagaagctattagcacaaatgtggaatttacaacaaacccatcagctaaattgcatccagtacatgtaagatccggctcgtgccccagtcagctgcagcacagtcaaccgctcaatgaaactccagcagccaagacagcggcacaacaacagcagttcctacccaagcagctgccttccgcttctacgaacagcgtttcgtacggatcgcaacgcgtgaaaggaagcaaggaacgtcctcacttgatttgcgcgagacaatcattagatgcagctacatttcgcagtatgtacaacaatgccgcgtcgccgccgccacctactacttccgtggccccagctgtctacgccggcggtcagcaacaggtgattcctggaggtggagcgcaatcccagttgcatgccaatatgattattgcacccagccatcggaagtcgcacagcctagatgcgagtcatgtgctgagtcccagcagcaatatgatcacggaggcggccattaaggccagcgccaccactaagtctccttactgcacgagggaaagtcgattccgctgcattgtgccgtatccaccaaacagtgacattgaactagagctacatttgggcgacattatctacgtccagcggaagcagaagaacggctggtataagggcacccatgcccgtacccacaaaaccgggctgttccccgcctcctttgttgaaccggattgttaggaaagttatggttcaaactagaatttattaagcgaaattccaaattacttgtctaaaaggattcaatcgtcggtctattcgggcttccaaatacgcaatctcatatttctcttttcaaaaaagaaaccgttttgtactcttccaatcgaatgggcagctcgccgttgtacttttttatacaatgcttgatcaaaataggctagccatgtaagacttagggaacagttacttaagccttagcgattagttagctagagaaataatctaaccgatccttgtgccctctacaaagttatttgtaatatacgatactcagtaataaaaaaaaaaaaaaaaaaaaaaaaaaa

SEQ ID NO: 11 - protein, 838 aa, Drosophila melanogaster
MDEHTLNDLLECSVCLERLDTTSKVLPCQHTFCRKCLQDIVASQHKLRCPECRILVSCKIDELPPNVLLMRILEGMKQNAAAGKGEEKGEETETQPERAKPQPPAESVAPPDNQLLQLQSHQQSHQPARHKQRRFLLPHAYALFDFASGEATDLKFKKGDLILIKHRIDNNWFVGQANGQEGTFPINYVKVSVPLPMPQCIAMYDFKMGPNDEEGCLEFKKSTVIQVMRRVDHNWAEGRIGQTIGIFPIAFVELNAAAKKLLDSGLHTHPFCHPPKQQGQRALPPVPVIDPTVVTESSSGSSNSTPGSSNSSSTSSSNNCSPNHQISLPNTPQHVVASGSASVRFRDKGAKEKRHSLNALLGGGAPLSLLQTNRHSAEILSLPHELSRLEVSSSTALKPTSAPQTSRVLKTTVQQQMQPNLPWGYLALFPYKPRQTDELELKKGCVYIVTERCVDGWFKGKNWLDITGVFPGNYLTPLRARDQQQLMHQWKYVPQNADAQMAQVQQHPVAPDVRLNNMLSMQPPDLPPRQQQATATTTSCSVWSKPVEALFSRKSEPKPETATASTTSSSSSGAVGLMRRLTHMKTRSKSPGASLQQVPKEAISTNVEFTTNPSAKLHPVHVRSGSCPSQLQHSQPLNETPAAKTAAQQQQFLPKQLPSASTNSVSYGSQRVKGSKERPHLICARQSLDAATFRSMYNNAASPPPPTTSVAPAVYAGGQQQVIPGGGAQSQLHANMIIAPSHRKSHSLDASHVLSPSSNMITEAAIKASATTKSPYCTRESRFRCIVPYPPNSDIELELHLGDIIYVQRKQKNGWYKGTHARTHKTGLFPASFVEPDC

SEQ ID NO: 12 - DNA, 18 bp, Artificial Sequence
  Description of Artificial Sequence Primer
cttgccttgccagcatac

SEQ ID NO: 13 - DNA, 18 bp, Artificial Sequence
  Description of Artificial Sequence Primer
ctgccagcattccttcag

SEQ ID NO: 14 - DNA, 21 bp, Artificial Sequence
  Description of Artificial Sequence Target sequence
aacagaggccttggaaacctg

SEQ ID NO: 15 - DNA, 21 bp, Artificial Sequence
  Description of Combined DNA/RNA Molecule siRNA
ttcagaggccuuggaaaccug

SEQ ID NO: 16 - DNA, 21 bp, Artificial Sequence
  Description of Combined DNA/RNA Molecule siRNA
ttcagguuuccaaggccucug

SEQ ID NO: 17 - DNA, 21 bp, Artificial Sequence
  Description of Artificial Sequence Target sequence
aaagagcctggagaccttaaa

SEQ ID NO: 18 - DNA, 21 bp, Artificial Sequence
  Description of Combined DNA/RNA Molecule siRNA
ttagagccuggagaccuuaaa

SEQ ID NO: 19 - DNA, 21 bp, Artificial Sequence
  Description of Combined DNA/RNA Molecule siRNA
ttuuuaaggucuccaggcucu

SEQ ID NO: 20 - DNA, 21 bp, Artificial Sequence
  Description of Artificial Sequence Target sequence
aaggattggtatgtgactctg

SEQ ID NO: 21 - DNA, 21 bp, Artificial Sequence
  Description of Combined DNA/RNA Molecule siRNA
ttggauugguaugugacucug

SEQ ID NO: 22 - DNA, 21 bp, Artificial Sequence
  Description of Combined DNA/RNA Molecule siRNA
ttcagagucacauaccaaucc

SEQ ID NO: 23 - DNA, 21 bp, Artificial Sequence
  Description of Artificial Sequence Target sequence
aagctggattatctcctgttg

SEQ ID NO: 24 - DNA, 21 bp, Artificial Sequence
  Description of Combined DNA/RNA Molecule siRNA
ttgcuggauuaucuccuguug

SEQ ID NO: 25 - DNA, 21 bp, Artificial Sequence
  Description of Combined DNA/RNA Molecule siRNA
ttcaacaggagauaauccagc

SEQ ID NO: 26 - protein, 41 aa, Artificial Sequence
  Description of Artificial Sequence RING domain
CPVCLERLDASAKVLPCQHTFCKRCLLGIVGSRNELRCPEC

SEQ ID NO: 27 - protein, 56 aa, Artificial Sequence
  Description of Artificial Sequence SH3 domain
PCAKALYNYEGKEPGDLKFSKGDIIILRRQVDENWYHGEVNGIHGFFPTNFVQIIK

SEQ ID NO: 28 - protein, 60 aa, Artificial Sequence
  Description of Artificial Sequence SH3 domain
PQCKALYDFEVKDKEADKDCLPFAKDDVLTVIRRVDENWAEGMLADKIGIFPISYVEFNS

SEQ ID NO: 29 - protein, 58 aa, Artificial Sequence
  Description of Artificial Sequence SH3 domain
SVYVAIYPYTPRKEDELELRKGEMFLVFERCQDGWFKGTSMHTSKIGVFPGNYVAPVT

SEQ ID NO: 30 - protein, 57 aa, Artificial Sequence
  Description of Artificial Sequence SH3 domain
ERHRVVVSYPPQSEAELELKEGDIVFVHKKREDGWFKGTLQRNGKTGLFPGSFVENI

SEQ ID NO: 31 - DNA, 121 bp, Artificial Sequence
  Description of Artificial Sequence RING domain
tgtccggtgtgtctagagcgccttgatgcttctgcgaaggtcttgccttgccagcatacgttttgcaagcgatgtttgctggggatcgtaggttctcgaaatgaactcagatgtcccgagt

SEQ ID NO: 32 - DNA, 165 bp, Artificial Sequence
  Description of Artificial Sequence SH3 domain
ccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattatt

SEQ ID NO: 33 - DNA, 177 bp, Artificial Sequence
  Description of Artificial Sequence SH3 domain
cctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaac

SEQ ID NO: 34 - DNA, 171 bp, Artificial Sequence
  Description of Artificial Sequence SH3 domain
agtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtc

SEQ ID NO: 35 - DNA, 169 bp, Artificial Sequence
  Description of Artificial Sequence SH3 domain
gaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaaca

SEQ ID NO: 36 - DNA, 21 bp, Artificial Sequence
  Description of Artificial Sequence Target sequence
aagtccaaaggttccggagac

SEQ ID NO: 37 - DNA, 1502 bp, Homo sapiens
agagacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - DNA, 2217 bp, Homo sapiens
gctgtgtggcccaggcttttctcaaactcctgagggcaagcgatcctcccacctcagcctcctgagtagctgggactacaggcatgtgccactagacctggctctaaagacatatatgacacacgaaaccatttatttttcatttcacaatgtttattcacatatatggtattagtattctaatgtagtgatgcactctaaatttgcattatatttcctagaacatctgaacagagcataggaaattccctattttgccattatcagttctaacaaaaatcttaaaagcactttatcatttcatttccctgcactgtaatttttttaaatgatcaaaaacagtatcataccaaggcttacttatattggaatactattttagaaagttgtgggctgggttgtatttataaatcttgttggtcagatgtctgcaatgagtaaatttagcaccattatcaggaagctttctcaccaatgacaacttcattggaagattttaatgaaagtgtagcatactctagggaaaaaatatgaatattttagcatctatgtattgaaaattatgttgaataaatgtcagactattttttacataacgttgcttctgtttaattttgtcacgttcagaggtggggggtaggagatgtaagcccttgacagcaaaataattccttttgcttgatttcagacagttgcatcagctcctttgttctgtgttcatgttacacttatttaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgacaaaaaaaaaaaaaaaaaa

SEQ ID NO: 39 - DNA, 1684 bp, Homo sapiens
ggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 40 - DNA, 1878 bp, Homo sapiens
ccacgcgtccgggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaa

SEQ ID NO: 41 - DNA, 1864 bp, Homo sapiens
aacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaaaaaaaaaaaaaaaaaa

SEQ ID NO: 42 - DNA, 1871 bp, Homo sapiens
gaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaaaaaaaaaaaaaaaaaaaa

SEQ ID NO: 43 - DNA, 1865 bp, Homo sapiens
agagacgtgaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 44 - DNA, 1884 bp, Homo sapiens
gacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 45 - DNA, 1860 bp, Homo sapiens
cgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtgtttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtaagatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 46 - DNA, 1884 bp, Homo sapiens; identical to SEQ ID NO: 44

SEQ ID NO: 47 - protein, 232 aa, Homo sapiens
MESETEPEPVTLLVKSPNQRHRDLELSGDRGWSVGHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 48 - protein, 209 aa, Homo sapiens
MQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 49 - protein, 356 aa, Homo sapiens
GHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 50 - protein, 391 aa, Homo sapiens
MESETEPEPVTLLVKSPNQRHRDLELSGDRGWSVGHLKAHLSRVYPERPRPEDQRLIYSGKLLLDHQCLRDLLPKQEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 51 - protein, 206 aa, Human immunodeficiency virus
MGGKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 52 - DNA, 21 bp, Artificial Sequence
  Description of Combined DNA/RNA Molecule siRNA
gggaaguucuucggaaccutt

SEQ ID NO: 53 - DNA, 21 bp, Artificial Sequence
  Description of Combined DNA/RNA Molecule siRNA
ttcccuucaagaagccuugga

SEQ ID NO: 54 - protein, 90 aa, Artificial Sequence
  Description of Artificial Sequence Consensus sequence
CXXCXXXXXXXXXXXXXXXXXXXXCXHXXXXXCXXCXXXXXXXXXXXXXXXXXXXXXXXXXXXXXXXXXXXXXXXXXXXXXXXXXXCXXC

SEQ ID NO: 55 - protein, 7 aa, Artificial Sequence
  Description of Artificial Sequence Consensus sequence
RXXPXXP

SEQ ID NO: 56 - protein, 4 aa, Artificial Sequence
  Description of Artificial Sequence Motif
PXAP

SEQ ID NO: 57 - protein, 5 aa, Artificial Sequence
  Description of Artificial Sequence Motif
PFRDY

SEQ ID NO: 58 - protein, 7 aa, Artificial Sequence
  Description of Artificial Sequence Motif
RPEPTAP

SEQ ID NO: 59 - protein, 7 aa, Artificial Sequence
  Description of Artificial Sequence Motif
RQGPKEP

SEQ ID NO: 60 - protein, 9 aa, Artificial Sequence
  Description of Artificial Sequence Motif
RQGPKEPFR

SEQ ID NO: 61 - protein, 9 aa, Artificial Sequence
  Description of Artificial Sequence Motif
RPEPTAPEE

SEQ ID NO: 62 - protein, 7 aa, Artificial Sequence
  Description of Artificial Sequence Motif
RPLPVAP

SEQ ID NO: 63 - protein, 7 aa, Artificial Sequence
  Description of Artificial Sequence Motif
RXXPXAP

SEQ ID NO: 64 - protein, 4 aa, Artificial Sequence
  Description of Artificial Sequence Motif
PTAP

SEQ ID NO: 65 - DNA, 53 bp, Artificial Sequence
  Description of Artificial Sequence Scrambled human POSH oligonucleotide
cacacactgccgtcaactgttcaagagacagttgacggcagtgtgtgtttttt

SEQ ID NO: 66 - DNA, 61 bp, Artificial Sequence
  Description of Artificial Sequence Scrambled human POSH oligonucleotide
aattaaaaaacacacactgccgtcaactgtctcttgaacagttgacggcagtgtgtgggcc

SEQ ID NO: 67 - DNA, 50 bp, Artificial Sequence
  Description of Artificial Sequence Oligonucleotide encoding RNAi against human POSH
aacagaggccttggaaacctggaagcttgcaggtttccaaggcctctgtt

SEQ ID NO: 68 - DNA, 54 bp, Artificial Sequence
  Description of Artificial Sequence Oligonucleotide encoding RNAi against human POSH
gatcaacagaggccttggaaacctgcaagcttccaggtttccaaggcctctgtt

SEQ ID NO: 69 - DNA, 29 bp, Artificial Sequence
  Description of Artificial Sequence Primer
ggcccactagtcaaggtcgggcaggaaga

SEQ ID NO: 70 - DNA, 48 bp, Artificial Sequence
  Description of Artificial Sequence Primer
gccgaattcaaaaaggatccggcgatatccggtgtttcgtcctttcca

SEQ ID NO: 71 - protein, 836 aa, Artificial Sequence
  Description of Artificial Sequence POSH fragment
RTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 72 - DNA, 1857 bp, Rattus sp.
aagacaccaagtgtcgttgtggggtcgcagacggctgcgtcgccgcccgttcggcatccctgagcgcagtcgagcctccagcgccgcagacatggagcccgagccacagcccgagccggtcacgctgctggtgaagagccccaatcagcgccaccgcgacttggagctgagtggcgaccgcggttggagtgtgagtcgcctcaaggcccacctgagccgagtctaccccgaacgcccgcgcccagaggaccagaggttaatttattctgggaagctgctgttggatcaccaatgtctccaagacttgcttccaaagcaggaaaagcgacatgttttgcacctcgtgtgcaatgtgaggagtccctcaaaaaagccagaagccagcacaaagggtgctgagtccacagagcagccggacaacactagtcaggcacagtatcctggggattcctcaagcgatggcttacgggaaagggaagtccttcggaaccttcctccctctggatgggagaacgtctctaggcctgaagccgtccagcagactttccaaggcctcgggcccggcttctctggctacaccacctacgggtggctgcagctctcctggttccagcagatctatgcaagacagtactacatgcaatacttggctgccactgctgcttcaggagcttttggccctacaccaagtgcacaagaaatacctgtggtctctacaccggctcccgcccctatacacaaccagtttccggcagaaaaccagccggccaatcagaatgcagccgctcaagcggttgttaatcccggagccaatcagaacttgcggatgaatgcacaaggcggccctctggtggaagaagatgatgagataaaccgagactggttggattggacctactcagcagcgacattttccgttttcctcagcattctttacttctactcctccctgagcagattcctcatggtcatgggcgccaccgtagtcatgtacctgcaccacgtcgggtggtttccattcagacagaggccagttcagaacttcccagatgacggtccccctcaggaagctgccaaccaggaccccaacaataacctccagggaggtttggaccctgaaatggaagaccccaaccgcctccccgtaggccgtgaagtgctggaccctgagcataccagcccctcgttcatgagcacagcatggctagtcttcaagactttctttgcctctcttcttccggaaggcccaccagccctagcaaactgatggcccctgtgctctgttgctggaggctttcacagcttggactggatcgtcccctggcgtggactcgagagagtcattgaaaacccacaggatgacgatgtgcttctgtgccaagcaaaagcacaaactaagacatgaagccgtggtacaaactgaacagggcccctcatgtcgttattctgaagagctttaatgtatactgtatgtagtctcataggcactgtaaacagaaggcccagggtcgcatgttctgcctgagcacctccccagacgtgtgtgcatgtgtgccgtacatggaagtcatagacgtgtgtgcatgtgtgctctacatggaagtcatagatgcagaaacggttctgctggttcgatttgattcctgttggaatgttgcaattacactaagtgtactactttatataatcagtgacttgctagacatgttagcaggacttttctaggagagacttattgtatcattgctttttaaaacgcagtgcttacttactgagggcggcgacttggcacaggtaaagcctttgccgggttttctgttcaataaagttttgctatgaacgacaaaaaaaaaaaaa

SEQ ID NO: 73 - protein, 391 aa, Rattus sp.
MEPEPQPEPVTLLVKSPNQRHRDLELSGDRGWSVSRLKAHLSRVYPERPRPEDQRLIYSGKLLLDHQCLQDLLPKQEKRHVLHLVCNVRSPSKKPEASTKGAESTEQPDNTSQAQYPGDSSSDGLREREVLRNLPPSGWENVSRPEAVQQTFQGLGPGFSGYTTYGWLQLSWFQQIYARQYYMQYLAATAASGAFGPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGPPQEAANQDPNNNLQGGLDPEMEDPNRLPVGREVLDPEHTSPSFMSTAWLVFKTFFASLLPEGPPALAN

SEQ ID NO: 74 - DNA, 1871 bp, Mus sp.
aaagacgccaagtgtcgttgtgtggtctcagacggctgcgtcgccgcccgttcggcatccctgagcgcagtcgagccgccagcgacgcagacatggagcccgagccacagcccgagccggtcacgctgctggtgaagagtcccaatcagcgccaccgcgacttggagctgagtggcgaccgcagttggagtgtgagtcgcctcaaggcccacctgagccgagtctaccccgagcgcccgcgtccagaggaccagaggttaatttattctgggaagctgctgttggatcaccagtgtctccaagatttgcttccaaagcaggaaaagcgacatgttttgcaccttgtgtgcaatgtgaagaatccctccaaaatgccagaaaccagcacaaagggtgctgaatccacagagcagccggacaactctaatcagacacagcatcctggggactcctcaagtgatggtttacggcaaagagaagttcttcggaacctttctccctccggatgggagaacatctctaggcctgaggctgtccagcagactttccaaggcctggggcctggcttctctggctacacaacgtatgggtggctgcagctctcctggttccagcagatctatgcaaggcagtactacatgcaatacttagctgccactgctgcatcaggaacttttgtcccgacaccaagtgcacaagagatacctgtggtctctacacctgctccggctcctatacacaaccagtttccggcagaaaaccagccggccaatcagaatgcagctgctcaagcggttgtcaatcccggagccaatcagaacttgcggatgaatgcacaaggtggccccctggtggaggaagatgatgagataaaccgagactggttggattggacctattccgcagcgacgttttctgttttcctcagcatcctttacttctactcctcgctgagcagatttctcatggtcatgggtgccactgtagtcatgtacctgcaccacgtcgggtggtttccgttcagacagaggccagttcagaacttcccggatgatggtggtcctcgagatgctgccaaccaggaccccaacaataacctccagggaggtatggacccagaaatggaagaccccaaccgcctccccccagaccgcgaagtgctggaccctgagcacaccagcccctcgtttatgagcacagcatggctagtcttcaagactttctttgcctctcttcttccagaaggcccaccagccctagccaactgatggcccttgtgctctgtcgctggtggctttgacagctcggactggatcgtctggctccggctccttttcctcccctggcgtggactcgacagagtcattgaaaacccacaggatgacatgtgcttctgtgccaagcaaaagcacaaactaagacatgaagccgtggtacaaactgaacagggcccctcatgtcgttattctgaagagctttaatgtatactgtatgtagtttcataggcactgtaagcagaaggcccagggtcgcatgttctgcctgagcacctccccagatgtgtgtgcatgtgtgctgtacatggaagtcatagacgtgtgtgcatgtgtgctctacatggaagtcatagatgcagaaacggttctgctggttcgatttgattcctgttggaatgttcaaattacactaagtgtactactttatataatcagtgaattgctagacatgttagcaggacttttctaggagagacttatgtataattgctttttaaaatgcagtgctttcctttaaaccgagggtggcgacttggcagaggtaaaacctttgccgagttttctgttcaataaagttttgctatgaatgactgt

SEQ ID NO: 75 - protein, 391 aa, Mus sp.
MEPEPQPEPVTLLVKSPNQRHRDLELSGDRSWSVSRLKAHLSRVYPERPRPEDQRLIYSGKLLLDHQCLQDLLPKQEKRHVLHLVCNVKNPSKMPETSTKGAESTEQPDNSNQTQHPGDSSSDGLRQREVLRNLSPSGWENISRPEAVQQTFQGLGPGFSGYTTYGWLQLSWFQQIYARQYYMQYLAATAASGTFVPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGGPRDAANQDPNNNLQGGMDPEMEDPNRLPPDREVLDPEHTSPSFMSTAWLVFKTFFASLLPEGPPALAN

SEQ ID NO: 76 - protein, 90 aa, Artificial Sequence
  Description of Artificial Sequence Consensus sequence
CXXCXXXXXXXXXXXXXXXXXXXXCXHXXXXXHXXCXXXXXXXXXXXXXXXXXXXXXXXXXXXXXXXXXXXXXXXXXXXXXXXXXXCXXC

(19) United States
(12) Patent Application Publication
(10) Pub. No.: US 2005/0214751 A1
(43) Pub. Date: Sep. 29, 2005

(54) Inhibition of viral maturation, methods and compositions related thereto

(75) Inventors: Yuval Reiss, Kiriat (IL); Iris Alroy, Ness-Ziona (IL)

Correspondence Address: FISH & NEAVE IP GROUP;ROPES & GRAY LLP, ONE INTERNATIONAL PLACE, BOSTON, MA, 02110-2624, US

(73) Assignees: Proteologics, Inc., Rehovot (IL); Proteologics, Ltd., Rehovot (IL)

(21) Appl. No.: 11/070,332

(22) Filed: Mar. 2, 2005

Related U.S. Application Data
(60) Provisional application No. 60/549,568, filed on Mar. 2, 2004.
(60) Provisional application No. 60/589,261, filed on Jul. 19, 2004.

(51) Int. Cl. C12Q 1/70

(52) U.S. Cl. 435/5

(57) ABSTRACT

The application relates to methods of inhibiting a Nef-mediated process in a cell infected with HIV. The application also provides methods and compositions for treating POSH-associated and HERPUD1-associated diseases such as viral disorders.

### RELATED APPLICATIONS

This application claims the benefit of priority of U.S. Provisional Application No. 60/549,568 entitled “Inhibition of Viral Maturation, Methods and Compositions Related Thereto” by Yuval Reiss and Iris Alroy, filed Mar. 2, 2004 and the benefit of priority of U.S. Provisional Application No. 60/589,261 entitled “Inhibition of Viral Maturation, Methods and Compositions Related Thereto” by Yuval Reiss and Iris Alroy, filed Jul. 19, 2004. The specifications of the referenced applications are incorporated herein by reference in their entirety.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However the discovery of relevant gene or protein targets is often difficult and time consuming.

The vesicular trafficking systems are the major pathways for the distribution of proteins among cell organelles, the plasma membrane and the extracellular medium. A central vesicular trafficking system involves the manipulation and passage of nascent proteins from the endoplasmic reticulum to the Golgi complex as well as the trafficking of protein complexes, including protein-nucleic acid complexes (e.g., protein-RNA, protein-DNA). The Golgi complex represents a major processing and sorting compartment for proteins destined for secretion or delivery to the cell surface or to lysosomes. A key component of this vesicular trafficking pathway is the trans-Golgi network (“TGN”). The Golgi complex is made up of multiple membrane-bound, flattened cisternae, and the TGN comprises the most distal of these. The individual cistemae or pairs of adjacent cisternae of the Golgi complex contain distinct sets of proteins. For example, the oligosaccharide-modification enzyme, galactosyltransferase, is selectively found within the trans-Golgi compartments. Additionally, the lipid composition changes from one side of the Golgi stack of cistemae to the other.

Manipulation and transport of proteins by the TGN involves proteins targeted to the TGN. In certain instances, the polypeptide ubiquitin is involved in these TGN trafficking events. For example, the transport of amino acid permeases, which are involved in the transport of amino acids into cells from the extracellular environment, is mediated by the TGN. The transport of amino acid permeases to either the plasma membrane or to the lysosome is determined in the TGN, and ubiquitination of these permeases has been implicated in their targeted transport from the TGN to the lysosome for degradation.

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, DNA repair, and intracellular transport. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and generally also requires auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin may then be transferred to a specific cysteine on one of several E2 enzymes. Finally, these E2 enzymes donate ubiquitin to protein substrates, typically with the assistance of an E3 protein, also known as a ubiquitin enzyme. In certain instances, substrates are recognized directly by the ubiquitin-conjugated E2 enzyme.

It is also known that the ubiquitin system plays a role in a wide range of cellular processes including cell cycle progression, apoptosis, and turnover of many membrane receptors. In viral infections, the ubiquitin system is involved not only with assembly, budding and release, but also with repression of host proteins such as p53, which may lead to a viral-induced neoplasm. The HIV Vpu protein interacts with an E3 protein that regulates IκB degradation and is thought to promote apoptosis of infected cells by indirectly inhibiting NF-κKB activity (Bour et al. (2001) J Exp Med 194:1299-311; U.S. Pat. No. 5,932,425). The ubiquitin system regulates protein function by both mono-ubiquitination and poly-ubiquitination, and poly-ubiquitination is primarily associated with protein degradation.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Viral maturation involves the proteolytic processing of the Gag proteins, organization of viral proteins and RNA to form a ribonucleoparticle, and the activity of various host proteins. It is believed that cellular machineries for exo/endocytosis and for ubiquitin conjugation may be involved in the maturation. In particular, the assembly, budding and subsequent release of retroid viruses, RNA viruses and envelope viruses, such as various retroviruses, rhabdoviruses, lentiviruses, and filoviruses may involve the Gag polyprotein. After its synthesis, Gag is targeted to the plasma membrane where it induces budding of nascent virus particles.

The role of ubiquitin in virus assembly was suggested by Dunigan et al. (1988, Virology 165, 310, Meyers et al. 1991, Virology 180, 602), who observed that mature virus particles were enriched in unconjugated ubiquitin. More recently, it was shown that proteasome inhibitors suppress the release of HIV-1, HIV-2 and virus-like particles derived from SIV and RSV Gag. Also, inhibitors affect Gag processing and maturation into infectious particles (Schubert et al 2000, PNAS 97, 13057, Harty et al. 2000, PNAS 97, 13871, Strack et al. 2000, PNAS 97, 13063, Patnaik et al. 2000, PNAS 97, 13069).

It would be beneficial to identify proteins involved in one or more of these processes for use in, among other things, drug screening methods.

### SUMMARY

The present application relates to an isolated, purified, or recombinant complex comprising a HERPUD1 polypeptide and a Nef polypeptide. In certain embodiments, the HERPUD1 polypeptide interacts directly with the Nef polypeptide. In certain further embodiments, the HERPUD1 polypeptide is a human HERPUD1 polypeptide.

In certain embodiments, the application relates to an isolated, purified, or recombinant complex comprising a HERPUD1 polypeptide and a Nef polypeptide, wherein the HERPUD1 polypeptide comprises an amino acid sequence that is at least 90% identical to an amino acid sequence selected from among SEQ ID NOS: 47-50 or any naturally occurring HERPUD1 amino acid sequence, and wherein the Nef polypeptide comprises an amino acid sequence that is at least 90% identical to a naturally occurring Nef amino acid sequence. In certain embodiments, the application relates to an isolated, purified, or recombinant complex comprising a HERPUD1 polypeptide and a Nef polypeptide, wherein the HERPUD1 polypeptide comprises an amino acid sequence that is at least 91%, 92%, 93%, 94%, 95%, 96%, 97%, 98%, or 99% identical to an amino acid sequence selected from among SEQ ID NOS: 47-50 or any naturally occurring HERPUD1 polypeptide, and wherein the Nef polypeptide comprises an amino acid sequence that is at least 91%, 92%, 93%, 94%, 95%, 96%, 97%, 98%, or 99% identical to a naturally occurring Nef polypeptide amino acid sequence. The HERPUD1 polypeptide can comprise a full length protein, or it can comprise a fragment of, for instance, at least 5, 10, 20, 50, 100, 150, 200, or more amino acids in length. The Nef polypeptide can comprise a full length protein, or it can comprise a fragment of, for instance, at least 5, 10, 20, 50, 100, 150, 200, or more amino acids in length.

In certain embodiments, the application relates to an isolated, purified, or recombinant complex comprising a HERPUD1 polypeptide and a Nef polypeptide, wherein the HERPUD1 polypeptide is encoded by a nucleic acid sequence that is at least 90% identical to a nucleic acid sequence selected from among SEQ ID NOS: 37-46 or any naturally occurring HERPUD1 nucleic acid sequence or a sequence complementary thereto, and wherein the Nef polypeptide is encoded by a nucleic acid sequence that is at least 90% identical to a naturally occurring Nef nucleic acid sequence or a sequence complementary thereto. In certain embodiments, the application relates to an isolated, purified, or recombinant complex comprising a HERPUD1 polypeptide and a Nef polypeptide, wherein the HERPUD1 polypeptide is encoded by a nucleic acid sequence that is at least 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of any of SEQ ID NOS: 37-46 or any naturally occurring HERPUD1 nucleic acid or a sequence complementary thereto, and wherein the Nef polypeptide is encoded by a nucleic acid sequence that is at least 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of a naturally occurring Nef nucleic acid sequence, or a sequence complementary thereto.

In certain embodiments, the application relates to an isolated, purified, or recombinant complex comprising a HERPUD1 polypeptide and a Nef polypeptide, wherein the HERPUD1 polypeptide is encoded by a nucleic acid sequence that hybridizes under stringent conditions to a nucleic acid sequence selected from among SEQ ID NOS: 37-46 or any naturally occurring HERPUD1 nucleic acid sequence, and wherein the Nef polypeptide is encoded by a nucleic acid sequence that hybridizes under stringent conditions to a naturally occurring Nef nucleic acid sequence. In certain embodiments, the application relates to an isolated, purified, or recombinant complex comprising a HERPUD1 polypeptide and a Nef polypeptide, wherein the HERPUD1 polypeptide is encoded by a nucleic acid sequence that hybridizes under stringent conditions to a nucleic acid sequence that is at least 90%, 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of any of SEQ ID NOS: 37-46 or any naturally occurring HERPUD1 nucleic acid or a sequence complementary thereto, and wherein the Nef polypeptide is encoded by a nucleic acid sequence that hybridizes under stringent conditions to a nucleic acid sequence that is at least 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of a naturally occurring Nef nucleic acid sequence or a sequence complementary thereto.

In other embodiments, the subject application relates to an in vitro reaction system comprising a HERPUD1 polypeptide and a Nef polypeptide. An in vitro reaction system includes any in vitro system suitable for carrying out an in vitro analysis of a HERPUD1 and/or Nef polypeptide. Examples of in vitro reaction systems include test tubes, microtiter plates, and Petri dishes, optionally comprising one or more aqueous solutions (e.g., buffers).

In additional embodiments, the application provides a method for identifying an agent that modulates (inhibits or potentiates) an activity of a HERPUD1 polypeptide, the method comprising identifying an agent that modulates (inhibits or potentiates) a complex comprising a HERPUD1 polypeptide and a Nef polypeptide, wherein an agent that modulates the complex comprising a HERPUD1 polypeptide and a Nef polypeptide is an agent that modulates an activity of the HERPUD1 polypeptide.

In further embodiments, the application provides a method of identifying an antiviral agent, comprising (a) identifying a test agent that modulates (inhibits or potentiates) a complex comprising a HERPUD1 polypeptide and a Nef polypeptide; and (b) evaluating the effect of the test agent on a function of a virus, wherein an agent that inhibits a pro-infective or pro-replicative function of a virus is an antiviral agent.

In other embodiments, the application provides a method of identifying an agent that modulates a Nef-mediated process, comprising identifying an agent that modulates HERPUD1 and testing the effect of the agent on a Nef-mediated process. In yet other embodiments, the application provides a method of evaluating an agent that modulates a Nef-mediated process, comprising providing an agent that modulates HERPUD1 and testing the effect of the agent on a Nef-mediated process. The effect of an agent on a Nef-mediated process may be tested by contacting a cell infected with HIV with the agent and measuring the effect of the agent on a Nef-mediated process. For example, the effect of an agent on a Nef-mediated process can be tested by testing its effect on the down-regulation of CD4 receptors; down-regulation of surface MHC class I molecules; enhancement of infectivity of HIV; and/or T cell activation in a cell infected with HIV. Examples of agents include an antibody, a small molecule, an RNAi construct, and an antisense construct. In certain embodiments, an agent inhibits a HERPUD1 activity. In other embodiments, an agent decreases the level of HERPUD1 polypeptide in a cell, for example, the level of HERPUD1 polypeptide in a cell infected with HIV. In other aspects, a HERPUD1 antagonist (e.g., an RNAi construct comprising SEQ ID NO: 52 or SEQ ID NO: 53) causes a decrease in the amount of Nef polypeptide in the cell.

In yet other embodiments, the application relates to a method of inhibiting viral replication in a cell, comprising contacting the cell with an agent that modulates (inhibits or potentiates) an interaction between a HERPUD1 polypeptide and a Nef polypeptide. In certain embodiments, the viral infection is caused by a human immunodeficiency virus, such as HIV-1.

In certain embodiments, the application relates to a method of inhibiting a viral infection in a subject in need thereof, comprising administering to the subject an agent that modulates (inhibits or potentiates) an interaction between a HERPUD1 polypeptide and a Nef polypeptide. In certain embodiments, the viral infection is caused by a human immunodeficiency virus. In certain further embodiments, the human immunodeficiency virus is HIV-1.

In certain embodiments, the methods of the subject invention employ a HERPUD1 polypeptide that is at least 90% identical to an amino acid sequence selected from among SEQ ID NOS: 47-50 or any naturally occurring HERPUD1 polypeptide. In certain embodiments, the HERPUD1 polypeptide comprises an amino acid sequence that is at least 91%, 92%, 93%, 94%, 95%, 96%, 97%, 98%, or 99% identical to an amino acid sequence selected from among SEQ ID NOS: 47-50 or any naturally occurring HERPUD1 polypeptide. The HERPUD1 polypeptide can comprise a full length protein, or it can comprise a fragment of, for instance, at least 5, 10, 20, 50, 100, 150, 200, or more amino acids in length.

In certain embodiments, the methods of the subject invention employ a HERPUD1 polypeptide that is encoded by a nucleic acid sequence that is at least 90% identical to a nucleic acid sequence selected from among SEQ ID NOS: 37-46 or any naturally occurring HERPUD1 nucleic acid. In certain embodiments, the HERPUD1 polypeptide is encoded by a nucleic acid sequence that is at least 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of any of SEQ ID NOS: 37-46 or any naturally occurring HERPUD1 nucleic acid, or a sequence complementary thereto.

In certain embodiments, the methods of the subject invention employ a HERPUD1 polypeptide that is encoded by a nucleic acid sequence that hybridizes under stringent conditions to a nucleic acid sequence selected from among SEQ ID NOS: 37-46 or any naturally occurring HERPUD1 nucleic acid. In certain embodiments, the HERPUD1 polypeptide is encoded by a nucleic acid sequence that hybridizes under stringent conditions to a nucleic acid sequence that is at least 90%, 91%, 92%, 93%, 94%, 95%, 96%, 97%, 98%, or 99% identical to a to a sequence corresponding to at least about 12, at least about 15, at least about 25, at least about 40, at least about 100, or at least about 300 consecutive nucleotides, up to the full length of any of SEQ ID NOS: 37-46 or any naturally occurring HERPUD1 nucleic acid or a sequence complementary thereto.

In certain embodiments, the methods of the subject invention employ a Nef polypeptide that is at least 90% identical to an amino acid sequence of a naturally occurring Nef polypeptide. In certain embodiments, the Nef polypeptide comprises an amino acid sequence that is at least 91%, 92%, 93%, 94%, 95%, 96%, 97%, 98%, or 99% identical to an amino acid sequence of a naturally occurring Nef polypeptide. The Nef polypeptide can comprise a full length protein, or it can comprise a fragment of, for instance, at least 5, 10, 20, 50, 100, 150, 200, or more amino acids in length. An example of a Nef polypeptide according to the subject application is depicted in SEQ ID NO: 51 and in the Examples.

In certain embodiments, the methods of the subject invention employ a Nef polypeptide that is encoded by a nucleic acid sequence that is at least 90% identical to a nucleic acid sequence selected from a naturally occurring Nef nucleic acid. In certain embodiments, the Nef polypeptide is encoded by a nucleic acid sequence that is at least 91%, 92%, 93%, 94%, 95%, 96%, 97%, 98%, or 99% identical to a nucleic acid sequence that is at least 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of a naturally occurring Nef nucleic acid sequence, or a sequence complementary thereto.

In certain embodiments, the methods of the subject invention employ a Nef polypeptide that is encoded by a nucleic acid sequence that hybridizes under stringent conditions to a nucleic acid sequence of a naturally occurring Nef nucleic acid. In certain embodiments, the Nef polypeptide is encoded by a nucleic acid sequence that hybridizes to a nucleic acid sequence that is at least 90%, 91%, 92%, 93%, 94%, 95%, 96%, 97%, 98%, or 99% identical to a sequence corresponding to at least about 12, at least about 15, at least about 25, at least about 40, at least about 100, or at least about 300 consecutive nucleotides, up to the full length of a naturally occurring Nef nucleic acid sequence, or a sequence complementary thereto.

Described herein are methods of inhibiting a Nef-mediated process in a cell infected with HIV. In preferred embodiments, the present application relates to a method of inhibiting a Nef-mediated process in a cell infected with HIV, comprising contacting the cell with a POSH antagonist, whereby a Nef-mediated process is inhibited in the cell.

In certain embodiments, a POSH antagonist of the application inhibits a POSH activity, for example, inhibits the ubiquitin ligase activity of a POSH polypeptide. In additional embodiments, a POSH antagonist decreases the level of POSH polypeptide in a cell, for example, the level of POSH polypeptide in a cell infected with HIV. In additional aspects, the application relates to POSH antagonists that inhibit one or more Nef-mediated processes. Exemplary Nef-mediated processes include the down modulation of CD4 receptors; the down modulation of MHC class I molecules; enhancement of infectivity of HIV; and T cell activation. In further embodiments, the MHC class I molecules are selected from among HLA-A and HLA-B.

In certain embodiments, a POSH antagonist of the application causes a decrease in the amount of Nef polypeptide in a cell infected with HIV. In certain embodiments, a POSH antagonist causes a decrease in the amount of Nef polypeptide that is membrane localized in a cell infected with HIV.

In certain embodiments, the methods of the application employ a cell infected with HIV, wherein the cell is situated in a subject that is infected with HIV. In further embodiments, the cell situated in the subject is contacted with a POSH antagonist by administration of the POSH antagonist to the subject. In other embodiments, the methods of the application employ a cell infected with HIV, wherein the cell is a cultured cell.

In further aspects, the application relates to measuring the effect of a POSH antagonist on a Nef-mediated process.

The present application also provides methods of inhibiting the progression of AIDS in a subject infected with HIV. In one embodiment, the application provides a method of inhibiting the progression of AIDS in a subject infected with HIV, comprising administering to the subject a POSH antagonist, whereby the progression of AIDS is inhibited in the subject. In certain aspects, the application relates to inhibiting the progression of AIDS in a subject infected with HIV, comprising administering a POSH antagonist, wherein the POSH antagonist inhibits a Nef-mediated process. In further embodiments, the application relates to inhibiting the progression of AIDS in a subject infected with HIV, comprising administering a POSH antagonist, wherein the progression of AIDS is inhibited by inhibiting a decline in CD4⁺ T-cell counts. In certain embodiments, inhibition results in no decline in CD4⁺ T-cell counts in the subject infected with HIV. AIDS is characterized by immunodeficiency, including a markedly reduced CD4⁺ T-cell count. In certain embodiments, the progression of AIDS in a subject infected with HIV is inhibited by mitigating the immunodeficiency in the subject by inhibiting a decline in CD4⁺ T-cell counts. Preferably, there is no decline in CD4⁺ T-cell counts.

In certain embodiments, the application relates to the use of a POSH antagonist for making a medicament for inhibiting a Nef-mediated process in an HIV infected cell.

In certain aspects, the application relates to agents that may be used in any of the various methods for affecting a Nef-mediated process. In certain embodiments, an agent is a POSH antagonist that is an RNAi construct, an antisense construct, an antibody or a small molecule. Optionally, an RNAi or antisense construct inhibits expression of a POSH polypeptide. In certain embodiments, an agent is a HERPUD1 antagonist that is an RNAi construct, an antisense construct, an antibody or a small molecule. Optionally, an RNAi or antisense construct inhibits expression of a HERPUD1 polypeptide.

In certain embodiments, a Nef-mediated process is inhibited in a subject infected with HIV by administration of a small molecule. Examples of small molecules include:

Compound CAS 27430-18-8:

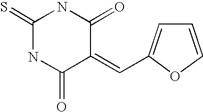

Compound CAS 1631-29-4:

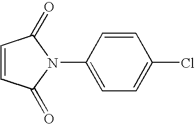

Compound CAS 503065-65-4:

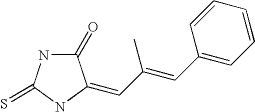

Compound CAS 414908-08:

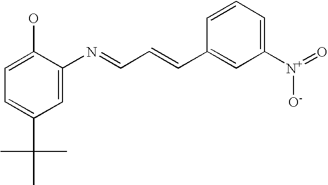

Compound CAS 415703-60-5:

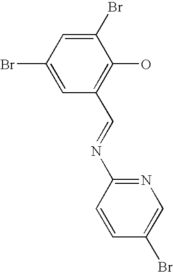

Compound CAS 77367-94-3:

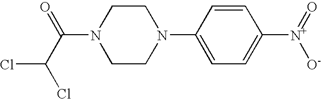

Compound CAS 154184-27-7:

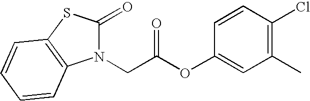

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1 shows human POSH coding sequence (SEQ ID NO:1).

FIG. 2 shows human POSH amino acid sequence (SEQ ID NO:2).

FIG. 3 shows human POSH cDNA sequence (SEQ ID NO:3).

FIG. 4 shows 5′ cDNA fragment of human POSH (public gi:10432611; SEQ ID NO:4).

FIG. 5 shows N terminus protein fragment of hPOSH (public gi:10432612; SEQ ID NO:5).

FIG. 6 shows 3′ mRNA fragment of hPOSH (public gi:7959248; SEQ ID NO:6).

FIG. 7 shows C terminus protein fragment of hPOSH (public gi:7959249; SEQ ID NO:7).

FIG. 8 shows human POSH full mRNA, annotated sequence.

FIG. 9 shows domain analysis of human POSH.

FIG. 10 is a diagram of human POSH nucleic acids. The diagram shows the full-length POSH gene and the position of regions amplified by RT-PCR or targeted by siRNA used in FIG. 11.

FIG. 11 shows effect of knockdown of POSH mRNA by siRNA duplexes. HeLa SS-6 cells were transfected with siRNA against Lamin A/C (lanes 1, 2) or POSH (lanes 3-10). POSH siRNA was directed against the coding region (153—lanes 3, 4; 155—lanes 5, 6) or the 3′UTR (157—lanes 7, 8; 159—lanes 9, 10). Cells were harvested 24 hours post-transfection, RNA extracted, and POSH mRNA levels compared by RT-PCR of a discrete sequence in the coding region of the POSH gene (see FIG. 10). GAPDH is used an RT-PCR control in each reaction.

FIG. 12 shows that POSH affects the release of VLP from cells. A) Phosphohimages of SDS-PAGE gels of immunoprecipitations of ³⁵S pulse-chase labeled Gag proteins are presented for cell and viral lysates from transfected HeLa cells that were either untreated or treated with POSH RNAi (50 nM for 48 hours). The time during the chase period (1, 2, 3, 4, and 5 hours after the pulse) are presented from left to right for each image.

FIG. 13 shows release of VLP from cells at steady state. Hela cells were transfected with an HIV-encoding plasmid and siRNA. Lanes 1, 3 and 4 were transfected with wild-type HIV-encoding plasmid. Lane 2 was transfected with an HIV-encoding plasmid which contains a point mutation in p6 (PTAP to ATAP). Control siRNA (lamin A/C) was transfected to cells in lanes 1 and 2. siRNA to Tsg101 was transfected in lane 4 and siRNA to POSH in lane 3.

FIG. 14 shows mouse POSH mRNA sequence (public gi:10946921; SEQ ID NO: 8).

FIG. 15 shows mouse POSH Protein sequence (Public gi:10946922; SEQ ID NO: 9).

FIG. 16 shows Drosophila melanogaster POSH mRNA sequence (public gi:17737480; SEQ ID NO:10).

FIG. 17 shows Drosophila melanogaster POSH protein sequence (public gi:17737481; SEQ ID NO:11).

FIG. 18 shows POSH domain analysis.

FIG. 19 shows that human POSH has ubiquitin ligase activity.

FIG. 20 shows that human POSH co-immunoprecipitates with RAC 1.

FIG. 21 shows effect of hPOSH on Gag-EGFP intracellular distribution.

FIG. 22 shows intracellular distribution of HIV-1 Nef in hPOSH-depleted cells.

FIG. 23 shows intracellular distribution of Src in hPOSH-depleted cells.

FIG. 24 shows intracellular distribution of Rapsyn in hPOSH-depleted cells.

FIG. 25 shows that knock-down of human POSH entraps HIV virus particles in intracellular vesicles. HIV virus release was analyzed by electron microscopy following siRNA and full-length HIV plasmid transfection. Mature viruses were secreted by cells transfected with HIV plasmid and non-relevant siRNA (control, bottom panel). Knockdown of Tsg101 protein resulted in a budding defect, the viruses that were released had an immature phenotype (top panel). Knockdown of hPOSH levels resulted in accumulation of viruses inside the cell in intracellular vesicles (middle panel).

FIG. 26 shows that phosphorylation of hPOSH regulates binding of GTP-loaded Rac-1. Bacterially expressed hPOSH (1 μg) (POSH) or GST (1 μg) (NS) were phosphorylated. Subsequently, GTPγS loaded or unloaded recombinant Rac-1 (0.2 μg) was added to hPOSH or GST. Bound racl was isolated as described in materials and methods and samples separated by SDS-PAGE on a 12% gel and immunobloted with anti-Rac-1. Input is 0.25 μg of Rac-1.

FIG. 27 shows that Nef protein levels (assessed by immunofluorescence) are undetectable in cell lines expressing RNAi directed against HPOSH while other viral proteins are expressed, for example env.

FIG. 28 shows that Nef protein levels (assessed by immunoblot) are undetectable in cell lines expressing RNAi directed against hPOSH.

FIG. 29 shows that siRNA-mediated reduction in HERPUD1 expression reduces HIV maturation.

FIG. 30 shows that endogenous Herp levels are reduced in H153 cells. H153 (POSH-RNAi) and H187 (control RNAi) cells were transfected with a plasmid encoding Flag-ubiquitin. Total cell lysates (A) or Flag-immunoprecipitated material (B) were separated on 10% SDS-PAGE and immunoblotted with anti-Herp antibodies.

FIG. 31 shows that exogenous Herp levels and its ubiquitination are reduced in POSH-depleted cells. H153 and H187 cells were co-transfected with Herp or control plasmids and a plasmid encoding Flag-ubiquitin (indicated above the figure). Total (A) and flag-immunoprecipitated material (B) were separated on 10% SDS-PAGE and immunoblotted with anti-Herp antibodies.

FIG. 32. HERPUD1 associates with HIV-1 Nef. Hela SS6 cells were transfected with plasmids encoding HERPUDI-Flag or Nef-Myc. Twenty-four hours post transfection cells were lysed and subjected to immunoprecipitation with anti-Flag antibodies. Cell lysates and immunoprecipitated material were separated by SDS-PAGE and immunoblotted with anti-Flag and anti-Myc antibodies (as indicated) to detect HERPUD1 or Nef, respectively.

FIG. 33. HERP depletion reduces Nef protein levels. Hela SS6 cells were transfected with siRNA directed against HERP and with a plasmid encoding HIV proviral genome (pNLenv-1). Twenty four hours post-HIV transfection, Nef protein levels were determined by immunoblot with anti-Nef specific antibodies.

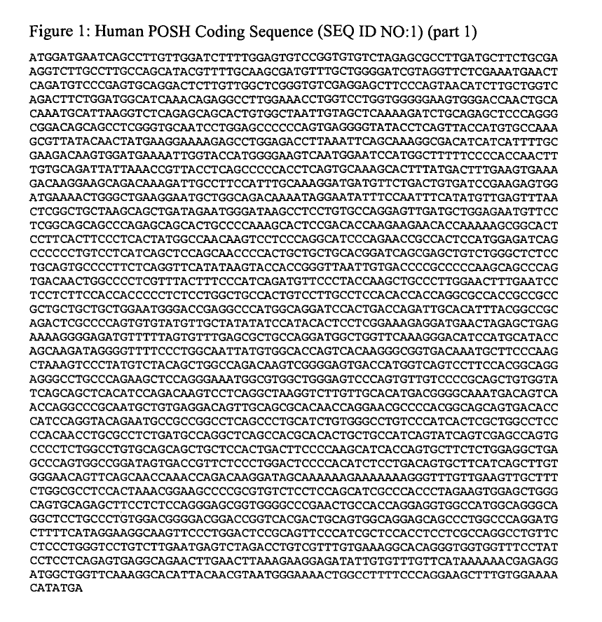

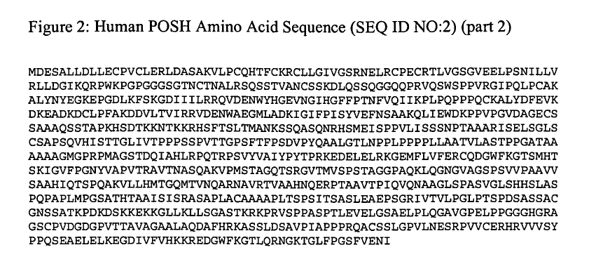

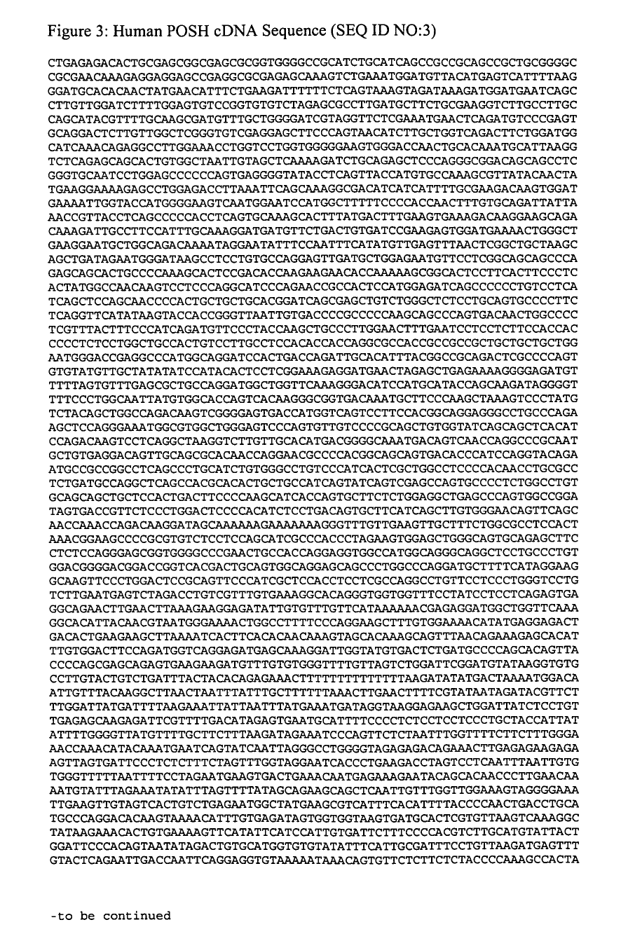

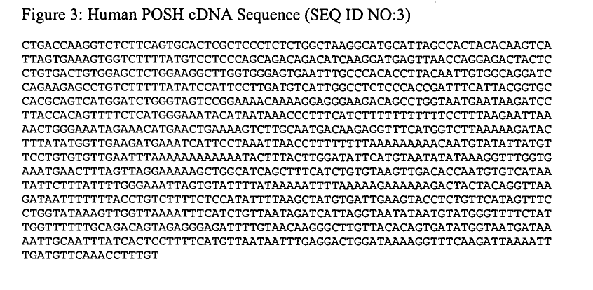

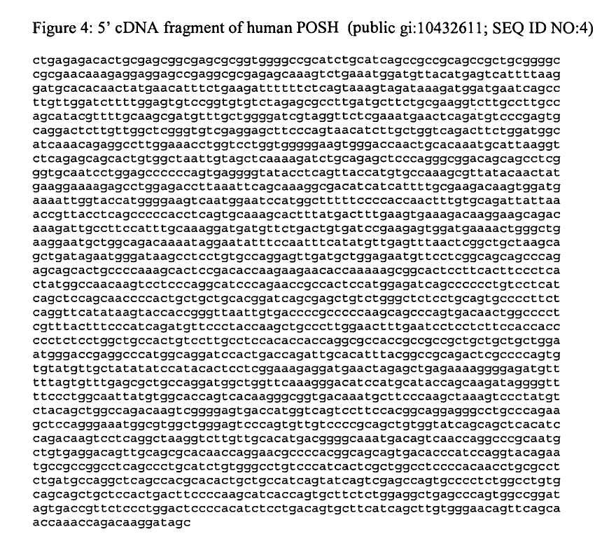

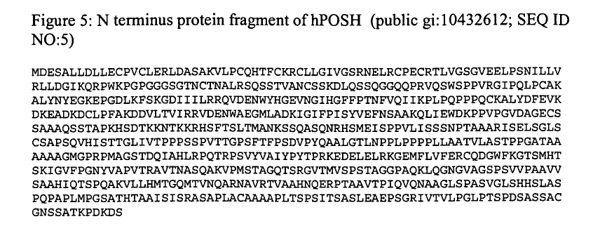

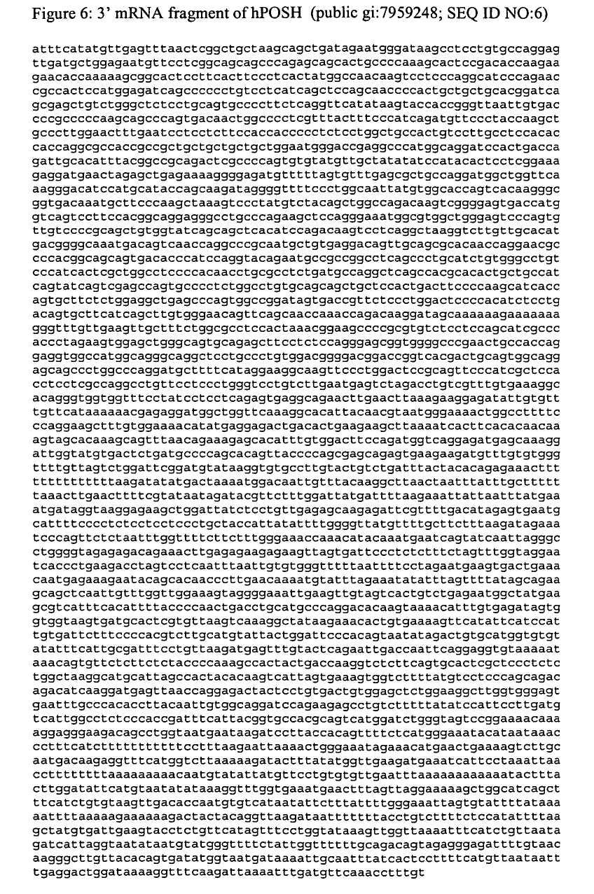

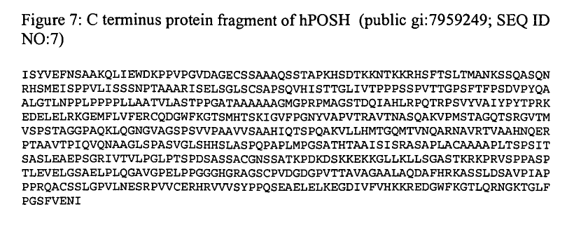

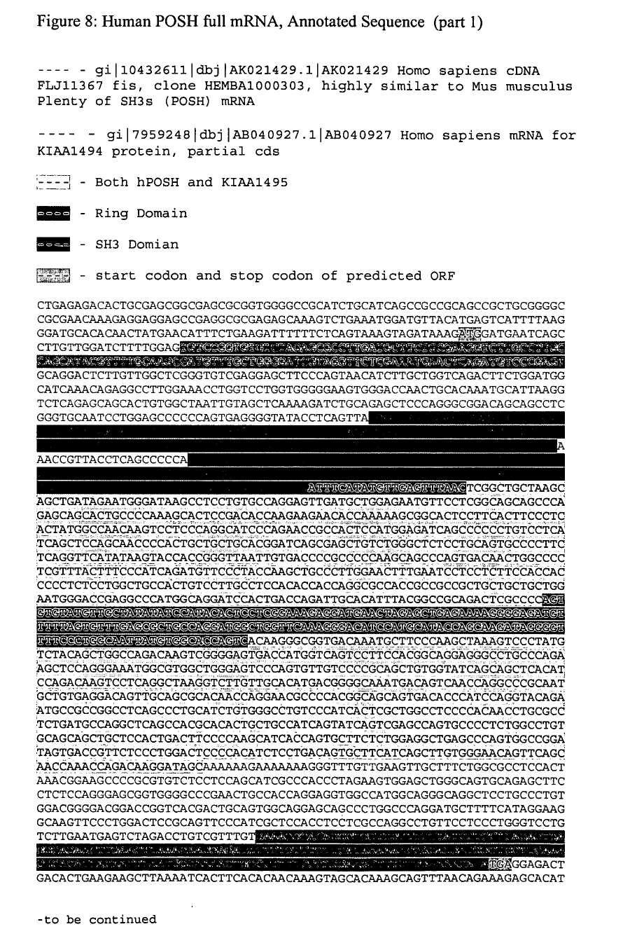

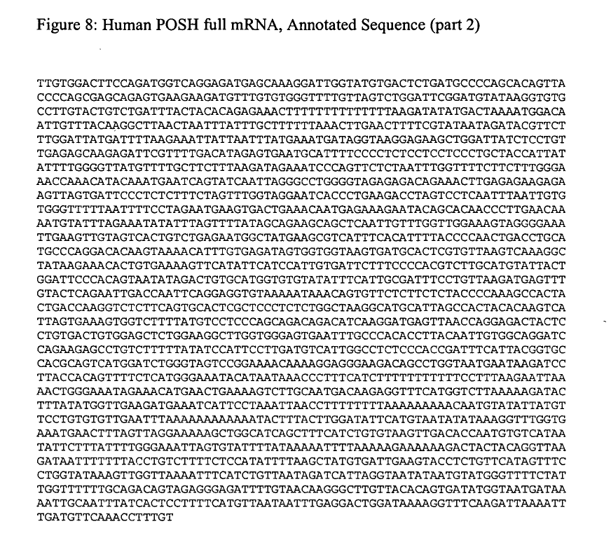

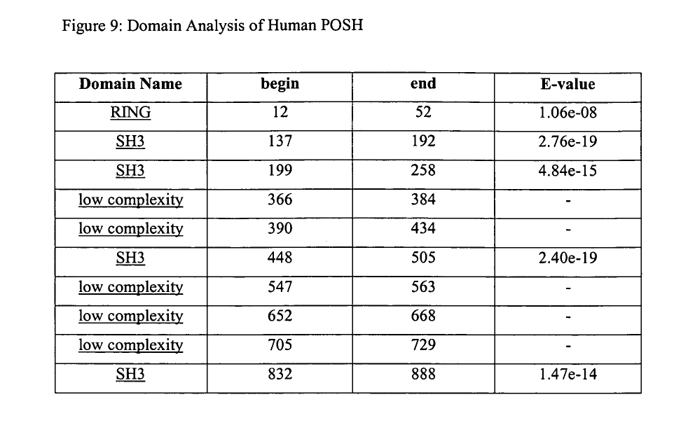

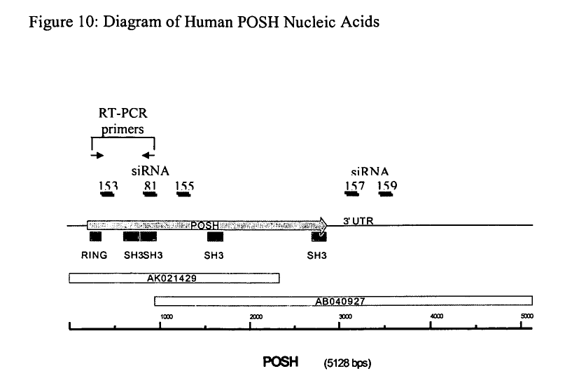

### DETAILED DESCRIPTION OF THE INVENTION

1. Definitions

The term “binding” refers to a direct association between two molecules, due to, for example, covalent, electrostatic, hydrophobic, ionic and/or hydrogen-bond interactions under physiological conditions.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “envelope virus” as used herein refers to any virus that uses cellular membrane and/or any organelle membrane in the viral release process.

A “HERPUD1-associated protein” or “HERPUD1-AP” refers to a protein capable of interacting with and/or binding to a HERPUD1 polypeptide. Generally, the HERPUD1-AP may interact directly or indirectly with the HERPUD1 polypeptide. Preferred HERPUD1-APs of the application include Nef and POSH.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present application may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the application. BLAST protein searches can be performed with the XBLAST program, score=50, wordlength=3 to obtain amino acid sequences homologous to protein molecules of the application. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

An “in vitro reaction system” refers to any in vitro system suitable for carrying out an in vitro analysis of one or more polypeptides. Examples of in vitro reaction systems include test tubes, microtiter plates, and Petri dishes, optionally comprising one or more aqueous solutions (e.g., buffers).

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

Lentiviruses include primate lentiviruses, e.g., human immunodeficiency virus types 1 and 2 (HIV-1/HIV-2); simian immunodeficiency virus (SIV) from Chimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (BIV); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M3843 1, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and U11820; 5)BIV. M32690; 6) E1AV: M16575, M87581 and U01866; 6) Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779.

The term “maturation” as used herein refers to the production, post-translational processing, assembly and/or release of proteins that form a viral particle. Accordingly, this includes the processing of viral proteins leading to the pinching off of nascent virion from the cell membrane.

As used herein, a “Nef-mediated process” is a cellular process involving Nef that impacts replication and maturation of a virus, such as, for example HIV (e.g., HIV-1, HIV-2) or SIV. A Nef-mediated process includes a process in which Nef interacts with host cell proteins and facilitates the production of infectious virions from the host cell. For example, a Nef-mediated process includes the down-regulation of CD4 levels from the cell surface. A Nef-mediated process additionally includes the down-modulation of cell surface major histocompatibility (MHC) class I molecules. Furthermore, Nef-mediated processes include T cell activation and enhancement of viral infectivity (e.g., HIV-1, HIV-2, or SIV viral infectivity).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

A “POSH nucleic acid” is a nucleic acid comprising a sequence as represented in any of SEQ ID Nos:1, 3, 4, 6, 8, and 10 as well as any of the variants described herein.

A “POSH polypeptide” or “POSH protein” is a polypeptide comprising a sequence as represented in any of SEQ ID Nos: 2, 5, 7, 9 and 11 as well as any of the variations described herein.

A “POSH-associated protein” or “POSH-AP” refers to a protein capable of interacting with and/or binding to a POSH polypeptide. Generally, the POSH-AP may interact directly or indirectly with the POSH polypeptide. A preferred POSH-AP of the application includes Nef. Other preferred POSH-APs include HERPUD1, PACS-1, HLA-A, and HLA-B.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present application which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Certain RING domains are represented as amino acid sequences that are at least 80% identical to amino acids 12-52 of SEQ ID NO: 2 and is set forth in SEQ ID NO: 26. Preferred RING domains are 85%, 90%, 95%, 98% and, most preferably, 100% identical to the amino acid sequence of SEQ ID NO: 26. Preferred RING domains of the application bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an RNAi construct. Phosphorothioate is a particularly common modification to the backbone of an RNAi construct. RNAi constructs include short hairpin RNA (shRNA) constructs.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the application.

An “SH3” or “Src Homology 3” domain is a protein domain of generally about 60 amino acid residues first identified as a conserved sequence in the non-catalytic part of several cytoplasmic protein tyrosine kinases (e.g., Src, Abl, Lck). SH3 domains mediate assembly of specific protein complexes via binding to proline-rich peptides. Exemplary SH3 domains are represented by amino acids 137-192, 199-258, 448-505 and 832-888 of SEQ ID NO:2 and are set forth in SEQ ID Nos: 27-30. In certain embodiments, an SH3 domain interacts with a consensus sequence of RXaaXaaPXaaX6P (where X6, as defined in table 1 below, is a hydrophobic amino acid). In certain embodiments, an SH3 domain interacts with one or more of the following sequences: P(T/S)AP, PFRDY, RPEPTAP, RQGPKEP, RQGPKEPFR, RPEPTAPEE and RPLPVAP.

As used herein, the term “specifically hybridizes” refers to the ability of a nucleic acid probe/primer of the application to hybridize to at least 12, 15, 20, 25, 30, 35, 40, 45, 50 or 100 consecutive nucleotides of a target gene sequence such as a POSH nucleic acid sequence or a HERPUD1 nucleic acid sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

A “virion” is a complete viral particle; nucleic acid and capsid (and a lipid envelope in some viruses. A “viral particle” may be incomplete, as when produced by a cell transfected with a defective virus (e.g., an HIV virus-like particle system).

TABLE 1

Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.embl-heidelberg.de/SMART_DATA/alignments/consensus/grouping.html.

2. Overview

In certain aspects, the application relates to the discovery of novel associations between HERPUD1 proteins and Nef proteins, and related methods and compositions. In certain aspects, the application relates to the discovery that a HERPUD1 polypeptide interacts with one or more Nef polypeptides. Accordingly, the application provides complexes comprising HERPUD1 and Nef. HERPUD1 is synonymous with Herp, and the terms are used interchangeably herein.

In certain embodiments, the application relates to the discovery of novel associations between POSH proteins and HERPUD1 proteins, and related methods and compositions. In further embodiments, the application relates to novel associations among certain disease states, POSH nucleic acids and proteins, and HERPUD1 nucleic acids and proteins.

In certain aspects, by identifying proteins associated with POSH, and particularly human POSH, the present application provides a conceptual link between the POSH-APs and cellular processes and disorders associated with POSH-APs, and POSH itself. Accordingly, in certain embodiments of the disclosure, agents that modulate a POSH-AP, such as HERPUD1, may now be used to modulate POSH functions and disorders associated with POSH function, such as viral disorders. Additionally, test agents may be screened for an effect on a POSH-AP, such as HERPUD1, and then further tested for an effect on a POSH function or a disorder associated with POSH function. Likewise, in certain embodiments of the disclosure, agents that modulate POSH may now be used to modulate a POSH-AP, such as HERPUDI, functions and disorders associated with POSH-AP function, such as disorders associated with HERPUD1 function, including HERPUD1-associated viral disorders . Additionally, test agents may be screened for an effect on HERPUD1 and then further tested for effect on a POSH-AP function or a disorder associated with POSH-AP function. In further aspects, the application provides nucleic acid agents (e.g., RNAi probes, antisense nucleic acids), antibody-related agents, small molecules and other agents that affect POSH function, and the use of same in modulating POSH and/or POSH-AP activity.

In certain aspects, the application relates to the discovery that a POSH polypeptide interacts with one or more HERPUD1 polypeptides. Accordingly, the application provides complexes comprising POSH and HERPUD1. In one aspect, the application relates to the discovery that POSH binds directly with HERPUD1. This interaction was identified by Applicants in a yeast 2-hybrid assay.

In certain aspects, the application relates to the discovery that a POSH polypeptide interacts with HERPUD1, a “homocysteine-inducible, endoplasmic reticulum stress-inducible, ubiquitin-like domain member 1” protein. In part, the present application relates to the discovery that the POSH-AP, HERPUD1, is involved in the maturation of an envelope virus, such as HIV.

HERPUD1 polypeptides are involved in JNK-mediated apoptosis, particularly in vascular endothelial cells, including cells that are exposed to high levels of homocysteine. HERPUD1 polypeptides are involved in the Unfolded Protein Response, a cellular response to the presence of unfolded proteins in the endoplasmic reticulum. HERPUD1 polypeptides are involved in the regulation of sterol biosynthesis. Accordingly, certain POSH polypeptides are involved in the Unfolded Protein Response and sterol biosynthesis.

In other aspects, certain HERPUD1 polypeptides enhance presenilin-mediated amyloid beta-protein generation. For example, HERPUD1 polypeptides, when overexpressed in cells, increase the level of amyloid beta generation, and it has been observed that HERPUD1 polypeptides interact with the presenilin proteins, presenilin-1 and presenilin-2. (See Sai, X. et al (2002) J. Biol. Chem. 277:12915-12920). The accumulation of amyloid beta is one hallmark of Alzheimer's disease. Accordingly, POSH polypeptides may be involved in the pathogenesis of Alzheimer's disease. At sites such as late intracellular compartment sites including the trans-Golgi network, certain mutant presenilin-2 polypeptides up-regulate production of amyloid beta peptides ending at position 42 (Aβ42). (See Iwata, H. et al (2001) J. Biol. Chem. 276: 21678-21685). Furthermore, elevated homocysteine levels have been found to be a risk factor associated with Alzheimer's disease and cerebral vascular disease.

The term HERPUD1 is used herein to refer as well to various naturally occurring HERPUD1 homologs, as well as functionally similar variants and fragments that retain at least 80%, 90%, 95%, or 99% sequence identity to a naturally occurring HERPUD1. The term specifically includes human HERPUD1 nucleic acid and amino acid sequences and the sequences presented in the Examples.

As described herein, POSH and HERPUD1 are involved in viral maturation, including the production, post-translational processing, assembly and/or release of proteins in a viral particle. Accordingly, viral infections may be ameliorated by inhibiting an activity of HERPUD1 and/or POSH (e.g., inhibition of POSH ubiquitin ligase activity), and in preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

In certain embodiments, the present application relates to the inhibition of a Nef-mediated process in a cell infected with HIV. In certain aspects, the application relates to a method of inhibiting a Nef-mediated process in a cell infected with HIV, comprising contacting the cell with a POSH antagonist, whereby a Nef-mediated process is inhibited in the cell. In certain further embodiments, the application relates to the inhibition of viral maturation by modulation of an activity associated with a Nef polypeptide, for example, by decreasing or preventing the expression of a POSH polypeptide and/or a HERPUD1 polypeptide.

Nef is a protein encoded by the nefaccessory gene of the HIV-1, HIV-2, and simian immunodeficiency virus (SIV). It is a 27 kDa, N-terminally myristoylated regulatory factor that is expressed at high levels shortly after HIV-1, HIV-2 and SIV viral infection. Nef is a major determinant of virus pathogenicity; it is important for achieving high viral loads and is important for viral replication and pathogenicity in humans in vivo (Deacon, N J et al (1995) Science 270:988-991; Kestler, H W III et al (1991) Cell 65:651-662; Mariani, R et al (1996) J Virol 70:7752-7764; Premkumar, D R et al (1996) 12:337-345; Lowe, S L et al (1996) 109:209-220).

Nef has been implicated in various cellular processes that impact HIV-1 viral replication and maturation, herein referred to as “Nef-mediated processes”. A Nef-mediated process includes the down-regulation of CD4 levels from the cell surface. A Nef-mediated process additionally includes the down-modulation of cell surface major histocompatibility (MHC) class I molecules. Furthermore, Nef-mediated processes include T cell activation and enhancement of HIV-1 viral infectivity.

Nef contributes to the ability of HIV to avoid detection by the host immune system. Nef does not have any enzymatic activity; it carries out its functions through protein-protein interactions with its targets and effector molecules, which are often part of trafficking or signaling pathways.

By down-regulating CD4, which is the primary receptor for HIV, Nef prevents super-infection of the host cell. Additionally, by decreasing CD4 expression at the cell surface, Nef enables the production of fully infectious virions from the host cell (Curr Biol (1999) 9:622-631). CD4 down-modulation by Nef involves endocytosis of CD4 followed by degradation via the endosomal/lysosomal pathway. This is accomplished by interactions between Nef and elements of the host cell endocytic machinery. For example, Nef facilitates connection of the cytoplasmic domain of CD4 with clathrin-coated pits at the plasma membrane (see, for example, J Cell Biol (1997) 139:37-47; J Virol (2001) 75:2488-2492; J Mol Biol (1994) 241:136-142). In certain embodiments of the application, inhibition of POSH (e.g., inhibition of the expression of POSH polypeptides) inhibits the Nef-mediated process of CD4 down-regulation. Accordingly, in certain embodiments of the application, POSH inhibition results in an increase in CD4 expression at the surface of a cell infected with HIV.

Nef also contributes to the ability of HIV to avoid detection by the immune system in that it is involved in the down-regulation of MHC class I molecules from the cell surface. Cells lacking surface MHC class I molecules are usually destroyed by natural killer (NK) cells. However, Nef mediates the down-regulation of the MHC class I molecules, HLA-A and HLA-B, but not that of HLA-C and HLA-E, which bind inhibitory receptors on NK cells. Nef-mediated down-modulation of MHC class I molecules has been reported to involve the diversion of these molecules from the cell surface towards endosomes and the trans-Golgi network (TGN). Furthermore, Nef induces the accumulation of HLA in the TGN. This effect has been shown to be blocked by inhibition of PI-3 kinase activity. Additionally, it has been shown that Nef interacts directly with phosphofurin acidic cluster sorting protein 1 (PACS-1). PACS-1 retrieves furin to the TGN. Further, Nef has been demonstrated to activate the GTPase, ARF6 after interacting with PACS-1. Nef interaction with ARF6 involves PI-3 kinase and the guanine nucleotide exchange factor, ARNO, and results in the activation of clathrin-independent routing of MHC class I molecules from the cell surface to an endosomal compartment followed by their retrieval to and trapping in the TGN. (See, for example, Virology (2001) 282:267-277; J Virol (2000) 74:9256-9266; EMBO J (2001) 20:2191-2201; Nature Cell Biol (2000) 2:163-167; Cell (2002) 111:853-866).

In certain embodiments of the application, inhibition of POSH (e.g., inhibition of the expression of POSH polypeptides) inhibits the Nef-mediated process of MHC class I molecule down-regulation. Accordingly, in certain embodiments of the application, POSH inhibition results in an increase in MHC class I molecule expression at the surface of a cell infected with HIV. In further embodiments, POSH inhibition, for example by use of a POSH antagonist that contacts a cell infected with HIV, results in an increase in the expression of HLA at the cell surface. In certain further embodiments, the HLA molecules are HLA-A and/or HLA-B.

POSH polypeptides have been shown to bind directly to the POSH-APs PACS-1, HLA-A, and HLA-B in a 2-hybrid assay. PACS-1 has been shown to bind to HIV Nef and is involved in the Nef-mediated process of HLA down-modulation from the surface of a cell infected with HIV. Accordingly, POSH may interact with Nef through its association with PACS-1. In certain aspects, POSH inhibition results in inhibition of PACS-1 activity, including inhibition of PACS-1 interaction with Nef and PACS-1 activity associated with Nef-mediated down-modulation of MHC class I molecules. In additional aspects, the application relates to inhibition of the down-regulation of HLA-A and HLA-B by Nef. In certain embodiments, POSH may interact with Nef through its interaction with HLA-A. In certain embodiments, POSH may interact with Nef through its interaction with HLA-B. In further embodiments of the application, POSH inhibition results in inhibition of HLA-A and/or HLA-B interaction with Nef.

To the extent that Nef down-modulates CD4 and MHC class I molecules, in certain embodiments, by inhibiting POSH or HERPUD1, CD4 and MHC class I molecule cell surface levels are accordingly increased.

For examples of CD4 downregulation assays, see Mariani, R et al. J. Virol. 70(11):7752-7764 (1996) and Chen, B K et al. J. Virol. 70(9):6044-6053 (1996). For examples of MHC class I internalization and recycling assays, see Blagoveshchenskaya, A D et al. Cell 111:853-866 (2002).

Another Nef-mediated process inhibited by methods of the present application is T cell activation. Nef has been implicated in T cell activation, for instance, in the production of IL-2. Its expression has been linked to the up-regulation of genes whose products are known to activate the HIV long terminal repeat (LTR), which contains enhancer and promoter sequences (J Virol (1999) 6094-6099; Immunity (2001) 14:763-777). Accordingly, T cell activation can be assessed by monitoring transcriptional changes in T cells (see e.g., Simmons, A et al. Immunity 14:763-777 (2001)). Additionally, Nef has been shown to form a complex with the cellular serine/threonine kinase p21-activated kinase 2 (Pak2) and to mediate Pak2 activation. Paks have been implicated in T cell activation. Accordingly, a Nef-mediated process includes Pak2 activation. (See, for example, Curr Biol (1999) 9:1407-1410; J Virol (2000) 74:11081-11087). In certain embodiments, inhibition of POSH or HERPUD1 (e.g., POSH or HERPUD1 polypeptide expression) results in inhibition of Pak2 activation. Nef has also been associated with nuclear factor of activated T cell (NFAT) transcriptional activity (J Virol (2001) 75:3034-3037). Additionally, Nef may associate with known activators of Paks, such as the Rho family GTPases, CDC42 and Rac 1, through its interaction with the guanine nucleotide exchange factor, Vav (or Vav2) (Mol Cell (1999) 3:729-739) or Pix (J Virol (1999) 73:9899-9907). In certain embodiments, POSH associates with the GTPase, Rac1. Accordingly, in certain aspects, POSH may interact with Nef through its association with Rac1. In certain embodiments, a POSH antagonist employed in the methods of the present application disrupts an interaction between POSH and Rac1. In further embodiments, this disruption results in an inhibition of the Nef-mediated process of Pak2 activation.

Nef-mediated processes further include enhancement of HIV infectivity. Single round infectivity assays demonstrate a Nef-dependent increase in productive target cell infection (see, for example, J Virol (1994) 68:2906-2914); J Virol (1995) 69:579-584). Regions in the C-terminus of Nef have been implicated in infectivity enhancement, and it is thought that this region of Nef plays a role in connecting Nef to the host cell trafficking machinery (J Virol (2000) 74:9836-9844; Virology (2000) 271:9-17; Mol Biol Cell (2001) 12:463-473). In certain embodiments of the present application, inhibition of the Nef-mediated process of enhancement of HIV-1 infectivity is accomplished by inhibiting a POSH polypeptide, such as, for example, by inhibiting the expression of POSH polypeptides in a cell infected with HIV or by contacting a cell infected with HIV with a POSH antagonist. In certain embodiments of the present application, inhibition of the Nef-mediated process of enhancement of HIV-1 infectivity is accomplished by inhibiting a HERPUD1 polypeptide, such as, for example, by inhibiting the expression of HERPUD1 polypeptides in a cell infected with HIV or by contacting a cell infected with HIV with a HERPUD1 antagonist.

Another Nef-mediated process relates to Nef involvement in protecting HIV-infected host cells from apoptosis, thereby allowing the HIV replicative cycle to go to completion. Nef has been shown to inhibit the apoptosis signal-regulating kinase 1 (ASK1) as well as the pro-apoptotic proteins BAD and p53 (see, for example, Nature (2001) 410:834-838; Nature Med (2001) 7:1217-1224; J Virol (2002) 76:2692-2702). Accordingly, in certain embodiments of the present application, a POSH antagonist induces apoptosis in a cell infected with HIV.

Nef has been shown to interact with SH3 domains, such as, for example, the SH3 domain of Vav, a CDC42/Rac guanine nucleotide exchange factor (Mol Cell (1999) 3:729-739) or the SH3 domain of Hck, a myeloid lineage-specific tyrosine kinase (Nature (1997) 385:650-653). Accordingly, Nef may interact with POSH SH3 domains.

Impaired Nef function has been associated with a lack of disease progression to AIDS (acquired immunodeficiency syndrome) in subjects infected with HIV. The progression of AIDS is characterized by a markedly reduced circulating CD4⁺ T-cell count. Furthermore, immunodeficiency is associated with AIDS, leading to opportunistic infections and tumors. Deletion in the Nef open reading frame, however, has been associated with a lack of disease progression in vivo. For example, infection of Rhesus monkeys with nef-deleted simian-immunodeficiency virus (SIV) did not lead to the development of AIDS-like disease and resulted in long-term immunity against pathogenic SIV (Daniel, M D et al (1992) Science 258:1938-1941; Kestler, H W III et al (1991) Cell 65:651-662). Analysis of HIV-1 positive human patients, which did not develop AIDS for 10-14 years, revealed mutations or deletions in the Nef coding sequence (Deacon, N J et al (1995) Science 270:988-991; Kestler, H W III et al (1991) Cell 65:651-662; Mariani, R et al (1996) J Virol 70:7752-7764; Premkumar, D R et al (1996) 12:337-345). Moreover, analysis of HIV-1 isolates from other asymptomatic patients found in the AIDS database revealed that they too harbor the same mutation in the Nef gene (Premkumar, D R et al (1996) 12:337-345). Importantly, patients that harbor Nef-mutated HIV-1 do not develop AIDS; Nef thus blocks HIV-1 pathogenicity.

Nef nucleic acid and the corresponding amino acid sequence encoded thereby are described in Chen, B K et al (1996) J Virol 70:6044-6053. Additional Nef nucleic acids and amino acids are referred to in Luo, T et al (1997) J Virol 71:9524-9530 (e.g, the Nef of HIV-1 isolate SF2 and the Nef of HIV-1 isolate 233) and Foster, J L et al (2001) J Virol 75:1672-1680. Nef nucleic acid and the corresponding amino acid sequence encoded thereby are also described by the SIV Nef described in Kestler, H W, III. et al (1991) Cell 65:651-662. The term Nef is used herein to refer as well to Nef of other HIV-1 isolates, as well as Nef of HIV-2 and other SIV isolates. The term Nef is used herein to refer to various naturally occurring Nef homologs, as well as functionally similar variants and fragments that retain at least 80%, 90%, 95%, or 99% sequence identity to a naturally occurring Nef. An example of a Nef polypeptide in accordance with the subject application is depicted in SEQ ID NO: 51.

In certain aspects, the application relates to modulation of a POSH process that is associated with the processing of Nef polypeptides. In certain embodiments, the application relates to the inhibition of a Nef-mediated process by inhibiting the membrane localization of Nef by inhibiting a POSH activity (e.g., ubiquitin ligase activity) or expression of POSH polypeptides in a cell infected with HIV. In certain embodiments, the application relates to the inhibition of a Nef-mediated process by inhibiting the expression of Nef polypeptides in a cell infected with HIV by inhibiting a POSH activity or expression of POSH polypeptides in the cell. The processing of Nef polypeptides includes the membrane localization of Nef, such as for example, the localization of Nef to membrane compartments of the host cell, including the perinuclear region (e.g., the TGN) and the plasma membrane. In further embodiments, processing of Nef polypeptides includes the proper myristoylation of the amino terminus of Nef. In certain embodiments, the application relates to the modulation of a POSH polypeptide, such as, for example, inhibition of the expression of a POSH polypeptide.

POSH intersects with and regulates a wide range of key cellular functions that may be manipulated by affecting the level of and/or activity of POSH polypeptides or POSH-AP polypeptides. Many features of POSH, and particularly human POSH, are described in PCT patent publications WO03/095971A2 (application no. WO2002US0036366) and WO03/078601A2 (application no. WO2003US0008194) the teachings of which are incorporated by reference herein.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself. POSH interacts with a large number of proteins and participates in a host of different biological processes. As demonstrated in this disclosure, POSH associates with a number of different proteins in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks. In addition, Applicants have demonstrated that POSH is necessary for proper secretion, localization or processing of a variety of proteins, including phospholipase D, HIV Gag, HIV Nef, Rapsyn and Src. Many of these proteins are myristoylated, indicating that POSH plays a general role in the processing and proper localization of myristoylated proteins. N-myristoylation is an acylation process, which results in covalent attachment of myristate, a 14-carbon saturated fatty acid to the N-terminal glycine of proteins (Farazi et al., J. Biol. Chem. 276: 39501-04 (2001)). N-myristoylation occurs co-translationaly and promotes weak and reversible protein-membrane interaction. Myristoylated proteins are found both in the cytoplasm and associated with membrane. Membrane association is dependent on protein configuration, i.e., surface accessibility of the myristoyl group may be regulated by protein modifications, such as phosphorylation, ubiquitination, etc. Modulation of intracellular transport of myristoylated proteins (e.g., Nef) in the application includes effects on transport and localization of these modified proteins. In preferred embodiments of the application, the application relates to the modulation of intracellular transport of the Nef.

As described herein, POSH and POSH-APs such as HERPUD1 are involved in viral maturation, including the production, post-translational processing, assembly and/or release of proteins in a viral particle. Accordingly, viral infections may be ameliorated by inhibiting an activity of POSH (e.g., ubiquitin ligase activity or target protein interaction) or a POSH-AP, such as HERPUD1 (e.g., associating with Nef), and in preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

As described in WO03/095971A2 (application no. WO2002US0036366) and WO03/078601A2 (application no. WO2003US0008194), POSH polypeptides function as E3 enzymes in the ubiquitination system. Accordingly, downregulation or upregulation of POSH ubiquitin ligase activity can be used to manipulate biological processes that are affected by protein ubiquitination. Modulation of POSH ubiquitin ligase activity may be used to affect POSH-APs and related biological processes, and likewise, modulation of POSH-APs may be used to affect POSH ubiquitin ligase activity and related processes. Downregulation or upregulation may be achieved at any stage of POSH formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity). As a further example, small molecule inhibitors of POSH ubiquitin ligase activity are provided herein. As another example, POSH activity may be increased by causing increased expression of POSH or an active portion thereof. POSH, and POSH-APs that modulate POSH ubiquitin ligase activity may participate in biological processes including, for example, one or more of the various stages of a viral lifecycle, such as viral entry into a cell, production of viral proteins, assembly of viral proteins and release of viral particles from the cell. POSH may participate in diseases characterized by excessive or inappropriate ubiquitination and/or protein degradation.

3. Methods and Compositions for Treatment of Viral-Related Disorders

Methods and compositions of the application for treatment of viral-related disorders that are associated with a POSH and/or HERPUD1 process contemplate inhibiting or preventing the onset or progression of the viral-related disorder, such as inhibiting or preventing the progression of AIDS in a subject infected with HIV. The methods and compositions of the application include means to screen for and identify therapeutic targets and/or drugs for the treatment of one or more viral-related disorders associated with a POSH and/or HERPUD1 process, such as HIV infection. Viral disorders associated with POSH and/or HERPUD1 processes include any viral disorder associated with a virus discussed above (e.g., HIV-1, HIV-2, or SIV).

In a further aspect, the invention provides methods and compositions for treatment of viral disorders, and particularly disorders caused by retroid viruses, RNA viruses and/or envelop viruses, including but not limited to retroviruses, rhabdoviruses, lentiviruses, and filoviruses. Preferred therapeutics of the invention function by disrupting the biological activity of a POSH and/or HERPUD1 protein that is involved in viral maturation. In a futher aspect, preferred therapeutics of the invention function by disrupting the biological activity of a complex comprising a POSH and/or HERPUD1 protein in viral maturation.

In view of the teachings herein, one of skill in the art will understand that the methods and compositions of the invention are applicable to a wide range of viruses such as for example retroid viruses, RNA viruses, and envelop viruses. In a preferred embodiment, the present invention is applicable to retroid viruses. In a more preferred embodiment, the present invention is further applicable to retroviruses (retroviridae). In another more preferred embodiment, the present invention is applicable to lentivirus, including primate lentivirus group. In another preferred embodiment, the present invention is applicable to flavivirus (flaviviridae), e.g., West Nile virus. In most preferred embodiments, the present invention is applicable to Human Immunodeficiency virus (HIV), Human Immunodeficiency virus type-1 (HIV-1), Hepatitis B Virus (HBV), Human T-cell Leukemia Virus (HTLV), West Nile virus.

While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus (EIAV), the C-type retrovirus which infects pigs, the Avian Leukosis Sarcoma Virus (ALSV), the Feline Leukemia Virus (FeLV), the Feline Aids Virus, the Bovine Leukemia Virus (BLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-II), Human Immunodeficiency virus type-2 (HIV-2) and Human Immunodeficiency virus type-1 (HIV-1).

The method and compositions of the present invention are further applicable to RNA viruses, including ssRNA negative-strand viruses and ssRNA positive-strand viruses. The ssRNA positive-strand viruses include Hepatitis C Virus (HCV) and flavivirus (e.g., West Nile virus). In a preferred embodiment, the present invention is applicable to mononegavirales, including filoviruses. Filoviruses further include Ebola viruses and Marburg viruses.

Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

4. POSH Antagonists

In certain aspects, the invention relates to methods employing compounds that affect a Nef-mediated process. In preferred embodiments of the application, the compounds are POSH antagonists. As described herein, a POSH antagonist is an agent that inhibits a POSH activity. In certain embodiments, a POSH antagonist may inhibit a POSH activity by inhibiting expression of the POSH polypeptide. In certain embodiments, a POSH antagonist may inhibit the ubiquitin ligase activity of a POSH polypeptide. In other preferred embodiments, the compounds inhibit a HERPUD1 activity by inhibiting expression of a HERPUD1 polypeptide. In other embodiments, the compounds may disrupt or render irreversible a complex comprising one or more of a HERPUD1 and a HERPUD1-AP, such as, for example, a HERPUD1-AP selected from among a Nef polypeptide and a POSH polypeptide. A HERPUD1 antagonist may be, for example, an RNAi construct, an antisense construct, an antibody, or a small molecule.

In certain further embodiments, a POSH antagonist may disrupt or render irreversible a complex comprising one or more of a POSH and a POSH-AP, such as, for example, a POSH-AP selected from among a Nef polypeptide, a PACS-1 polypeptide, an HLA-A polypeptide, and an HLA-B polypeptide. In certain additional embodiments, a POSH antagonist interferes with the interaction between POSH and a POSH-AP polypeptide, for example a POSH antagonist may disrupt or render irreversible the interaction between a POSH polypeptide and POSH-AP polypeptide such as another POSH polypeptide (as in the case of a POSH dimer, a heterodimer of two different POSH, homomultimers and heteromultimers); Cbl-b; an MSTP028; a HERPUD1; a GTPase (eg. Rac, Rac1, Rho, Ras); an E2 enzyme; ubiquitin; PAK1, PAK2, PAK family, Vav, Cdc42, PI3K (e.g., p85 or p110), a Gag, particularly an HIV Gag (e.g., p160); Vpu; as well as, in certain embodiments, proteins known to be associated with clathrin-coated vesicles and/or proteins involved in the protein sorting pathway. A POSH antagonist may be, for example, an RNAi construct, an antisense construct, an antibody, or a small molecule.

In certain embodiments, a small molecule is employed, such as a small molecule selected from among:

Compound CAS 27430-18-8:

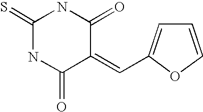

Compound CAS 1631-29-4:

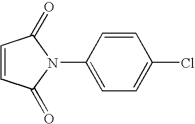

Compound CAS 503065-65-4:

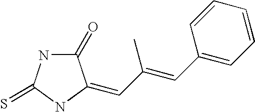

Compound CAS 414908-08:

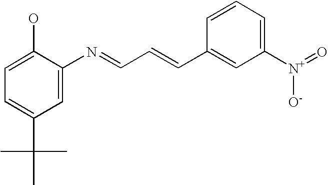

Compound CAS 415703-60-5:

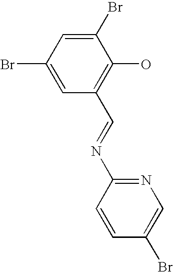

Compound CAS 77367-94-3:

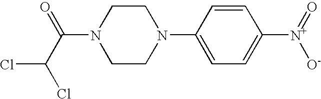

Compound CAS 154184-27-7:

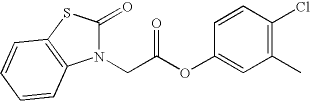

In certain embodiments, compounds useful in the instant compositions and methods include heteroarylmethylene-dihydro-2,4,6-pyrimidinetriones and their thione analogs. Preferred heteroaryl moieties include 5-membered rings such as thienyl, furyl, pyrrolyl, oxazolyl, thiazolyl, and imidazolyl moieties.

In certain embodiments, compounds useful in the instant compositions and methods include N-arylmaleimides, especially N-phenylmaleimides, in which the phenyl group may be substituted or unsubstituted.

In certain embodiments, compounds useful in the instant compositions and methods include arylallylidene-2,4-imidazolidinediones and their thione analogs. Preferred aryl groups are phenyl groups, and both the aryl and allylidene portions of the molecule may be substituted or unsubstituted.

In certain embodiments, compounds useful in the instant compositions and methods include substituted distyryl compounds and aza analogs thereof such as substituted 1,4-diphenylazabutadiene compounds.

In certain other embodiments, compounds useful in the instant compositions and methods include substituted styrenes and aza analogs thereof, such as 1,2-diphenylazaethylenes and 1-phenyl-2-pyridyl-azaethelenes.

In yet other embodiments, compounds useful in the instant compositions and methods include N-aryl-N′-acylpiperazines. In such compounds, the aryl ring, the acyl substituent, and/or the piperazine ring may be substituted or unsubstituted.

In additional embodiments, compounds useful in the instant compositions and methods include aryl esters of (2-oxo-benzooxazol-3-yl)-acetic acid, and analogs thereof in which one or more oxygen atoms are replaced by sulfur atoms.

Optionally, a small molecule inhibits the ubiquitin ligase activity of a POSH polypeptide.

In certain embodiments, the present application relates to the use of a POSH antagonist to inhibit a Nef-mediated process. In certain embodiments, a POSH antagonist causes a decrease in the amount of Nef polypeptide in an HIV infected cell. In certain embodiments, a POSH antagonist causes a descrease in the amount of Nef polypeptide that is membrane localized (e.g., localized to the perinuclear region) in an HIV infected cell.

The application further relates to measuring the effect of a POSH antagonist on a Nef-mediated process. This includes measuring the effect of different dosing regimens of a POSH antagonist on a Nef-mediated process, such as, for example, measuring the effect of a given dose of a POSH antagonist on the CD4⁺ T cell count in a subject infected with HIV. In certain embodiments, a POSH antagonist inhibits the progression of AIDS in a subject infected with HIV by inhibiting a decline in CD4⁺ T cell counts. In certain embodiments of the present application, a POSH antagonist increases CD4⁺ T cell counts in a subject diagnosed with AIDS, for example, in a subject infected with HIV that has progressed to AIDS, for example, in a subject manifesting the clinical symptoms of AIDS, such as markedly reduced CD4⁺ T cell counts. In certain embodiments, a POSH antagonist increases CD4 cell surface expression in a cell infected with HIV. In certain embodiments, a POSH antagonist increases MHC class I molecule expression, such as for example, HLA-A and HLA-B expression, at the cell surface in a cell infected with HIV. In certain embodiments, a POSH antagonist inhibits T cell activation. In certain additional embodiments, a POSH antagonist inhibits HIV infectivity.

A POSH antagonist may inhibit a Nef-mediated process by contacting a cell infected with HIV in a subject in need thereof, wherein a Nef-mediated process is inhibited in the subject. A POSH antagonist may be administered to one or more subjects infected with HIV.

The POSH antagonists useful in the methods of the present application may be administered to any subject in need thereof, for example, to a subject infected with HIV.

5. RNA Interference, Ribozymes, Antisense and DNA Enzyme

In certain aspects, the invention relates to RNAi, ribozyme, antisense and other nucleic acid-related methods and compositions for manipulating (typically decreasing) a POSH and/or HERPUD1 protein activity. Exemplary RNAi and ribozyme molecules may comprise a sequence as shown in any of SEQ ID NOS: 15, 16, 18, 19, 21, 22, 24, 25, 52 and 53.

Certain embodiments of the invention make use of materials and methods for effecting knockdown of one or more protein genes, such as POSH or HERPUD1, by means of RNA interference (RNAi), using an RNAi construct. RNAi is a process of sequence-specific post-transcriptional gene repression which can occur in eukaryotic cells. In general, this process involves degradation of an mRNA of a particular sequence induced by duplex containing an antisense RNA that hybridizes to the target sequence. An RNAi construct may, for example, be a double stranded RNA (dsRNA), a DNA:RNA hybrid or a hairpin RNA comprising a short duplex region. In general, the sense portion of the duplex is amenable to modifications, while the antisense portion should generally be unmondified RNA or mostly unmodified RNA. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the niRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of a gene in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discemable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. Nos. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art—e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the protein gene product following sufficient time for turnover of the endogenous pool after new protein synthesis is repressed, reverse transcriptase polymerase chain reaction and Northern blot analysis to determine the level of existing protein target mRNA.

Further compositions, methods and applications of RNAi technology are provided in U.S. Pat. Nos. 6,278,039, 5,723,750 and 5,244,805, which are incorporated herein by reference.

Ribozyme molecules designed to catalytically cleave protein (e.g., POSH, HERPUD1) mRNA transcripts can also be used to prevent translation of subject protein (e.g., POSH, HERPUD1) mRNAs and/or expression of protein (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a POSH or HERPUD1 protein mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target protein mRNA, such as an mRNA of a sequence represented in any of SEQ ID NOS: 1, 3, 4, 6, 8, or 10 or 37-46. In addition, ribozymes possess highly specific endoribonuclease activity, which autocatalytically cleaves the target sense mRNA. The present invention extends to ribozymes which hybridize to a sense mRNA encoding a POSH or HERPUD1 protein gene such as a therapeutic drug target candidate gene, thereby hybridizing to the sense mRNA and cleaving it, such that it is no longer capable of being translated to synthesize a functional polypeptide product.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a POSH or HERPUD1 protein nucleic acid, such as a nucleic acid of any of SEQ ID NOS: 1, 3, 4, 6, 8, or 10 or 37-46. In a long target RNA chain, significant numbers of target sites are not accessible to the ribozynie because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (seeMilner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

A further aspect of the invention relates to the use of the isolated “antisense” nucleic acids to inhibit expression, e.g., by inhibiting transcription and/or translation of a subject POSH protein nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a POSH or HERPUD1 polypeptide. Alternatively, the antisense construct is an oligonucleotide probe, which is generated ex vivo and which, when introduced into the cell causes inhibition of expression by hybridizing with the mRNA and/or genomic sequences of a POSH or HERPUD1 protein nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

With respect to antisense DNA, oligodeoxyribonucleotides derived from the translation initiation site, e.g., between the −10 and +10 regions of the POSH or HERPUD1 protein gene, are preferred. Antisense approaches involve the design of oligonucleotides (either DNA or RNA) that are complementary to mRNA encoding the target polypeptide (e.g., POSH, HERPUD1). The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a target protein (e.g., POSH, HERPUD1) mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, protein gene expression, e.g., POSH protein gene expression or HERPUD1 protein gene expression, can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine- rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential POSH or HERPUD1 protein sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of a POSH or HERPUD1 protein gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

7. Exemplarv Nucleic Acids and Expression Vectors

In certain aspects, the methods of the invention provide nucleic acids encoding POSH polypeptides. In certain aspects, the methods of the invention provide nucleic acids encoding POSH polypeptides, such as, for example, SEQ ID NOS: 2, 5, 7, 9, 11, 26, 27, 28, 29 and 30. Nucleic acids of the invention are further understood to include nucleic acids that comprise variants of SEQ ID NOS: 1, 3, 4, 6, 8, 10, 31, 32, 33, 34, and 35. Variant nucleotide sequences include sequences that differ by one or more nucleotide substitutions, additions or deletions, such as allelic variants and will, therefore, include coding sequences that differ from the nucleotide sequence of the coding sequence designated in SEQ ID NOS: 1, 3, 4, 6, 8 10, 31, 32, 33, 34, and 35, e.g., due to the degeneracy of the genetic code. In other embodiments, variants will also include sequences that will hybridize under highly stringent conditions to a nucleotide sequence of a coding sequence designated in any of SEQ ID Nos:1, 3, 4, 6, 8 10, 31, 32, 33, 34, and 35. Preferred nucleic acids of the invention are human POSH sequences, including, for example, any of SEQ ID Nos: 1, 3, 4, 6, 31, 32, 33, 34, 35 and variants thereof and nucleic acids encoding an amino acid sequence selected from among SEQ ID Nos: 2, 5, 7, 26, 27, 28, 29 and 30.

In certain aspects, the methods of the invention provide nucleic acids encoding HERPUD1 polypeptides. In certain aspects, the methods of the invention provide nucleic acids encoding HERPUD1 polypeptides, such as, for example, SEQ ID NOS: 37-46 and those found in the Examples. Nucleic acids of the invention are further understood to include nucleic acids that comprise variants of SEQ ID NOS: 37-46. Variant nucleotide sequences include sequences that differ by one or more nucleotide substitutions, additions or deletions, such as allelic variants and will, therefore, include coding sequences that differ from the nucleotide sequence of the coding sequences designated in SEQ ID NOS: 37-46, e.g., due to the degeneracy of the genetic code. In other embodiments, variants will also include sequences that will hybridize under highly stringent conditions to a nucleotide sequence of a coding sequence designated in any of SEQ ID NOS: 37-46. Preferred nucleic acids of the invention are human HERPUD1 sequences, including, for example, any of SEQ ID NOS: 37-46 and variants thereof and nucleic acids encoding an amino acid sequence selected from among SEQ ID NOS: 47-50.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from SEQ ID NOS:1, 3, 4, 6, 8, 10, 31, 32, 33, 34, 35, and 37-46 due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

Optionally, a POSH or HERPUD1 nucleic acid of the invention will genetically complement a partial or complete POSH or HERPUD1 protein loss of function phenotype in a cell. For example, a POSH nucleic acid of the invention may be expressed in a cell in which endogenous POSH has been reduced by RNAi, and the introduced POSH nucleic acid will mitigate a phenotype resulting from the RNAi. An exemplary POSH loss of function phenotype is a decrease in virus-like particle production in a cell transfected with a viral vector, optionally an HIV vector. In certain embodiments, a POSH nucleic acid, when expressed at an effective level in a cell, induces apoptosis.

Another aspect of the invention relates to POSH and HERPUD1 nucleic acids that are used for antisense, RNAi or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the subject POSH polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present invention can be delivered, for example, as an expression plasmid which, when transcribed in the cell, produces RNA which is complementary to at least a unique portion of the cellular mRNA which encodes a POSH or HERPUD1 protein. Alternatively, the construct is an oligonucleotide which is generated ex vivo and which, when introduced into the cell causes inhibition of expression by hybridizing with the mRNA and/or genomic sequences encoding a POSH or HERPUD1 protein. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, the modified oligomers of the invention are useful in therapeutic, diagnostic, and research contexts. In therapeutic applications, the oligomers are utilized in a manner appropriate for nucleic acid therapy in general.

In addition to use in therapy, the oligomers of the invention may be used as diagnostic reagents to detect the presence or absence of the POSH or HERPUD1 DNA or RNA sequences to which they specifically bind, such as for determining the level of expression of a gene of the invention or for determining whether a gene of the invention contains a genetic lesion.

In another aspect of the invention, the subject nucleic acid is provided in an expression vector comprising a nucleotide sequence encoding a subject POSH or HERPUD1 protein and operably linked to at least one regulatory sequence. Regulatory sequences are art-recognized and are selected to direct expression of the POSH or HERPUD1 protein. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding POSH or HERPUD1 protein. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda , the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject proteins in cells propagated in culture, e.g., to produce proteins or polypeptides, including fusion proteins or polypeptides, for purification.

This invention also pertains to a host cell transfected with a recombinant gene including a coding sequence for one or more of the subject proteins. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject proteins (e.g., POSH, HERPUD1, Nef). For example, a host cell transfected with an expression vector encoding a polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In one embodiment, the subject protein is a fusion protein containing a domain which facilitates its purification, such as a POSH protein-GST fusion protein, -intein fusion protein, -cellulose binding domain fusion protein, -polyhistidine fusion protein, etc.

A nucleotide sequence encoding a subject protein, such as POSH, HERPUD1, and/or Nef, can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant nucleic acid can be produced by ligating the cloned gene (e.g., POSH, HERPUD1, Nef), or a portion thereof, into a vector suitable for expression in either prokaryotic cells, eukaryotic cells, or both. Expression vehicles for production of a recombinant protein include plasmids and other vectors. For instance, suitable vectors for the expression of a POSH protein include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a POSH protein. For example, the VP6 capsid protein of rotavirus can be used as an immunologic carrier protein for portions of polypeptide, either in the monomeric form or in the form of a viral particle. The nucleic acid sequences corresponding to the portion of the POSH protein to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a POSH protein and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a protein, such as a POSH or HERPUD1 protein, is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a protein can also be expressed and presented by bacterial cells.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified protein (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

TABLE 2
Exemplary POSH nucleic acids

| | | Accession |
|---|---|---|
| Sequence Name | Organism | Number |
| cDNA FLJ11367 fis, | Homo sapiens | AK021429 |
| clone HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 3
Exemplary POSH polypeptides

| | | Accession |
|---|---|---|
| Sequence Name | Organism | Number |
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

In addition the following Tables provide the nucleic acid sequence and related SEQ ID NOs for domains of human POSH protein and a summary of sequence identification numbers used for POSH in this application.

TABLE 4

Nucleic Acid Sequences and related SEQ ID NOs for domains
in human POSH

| Name of the | | SEQ ID |
|---|---|---|
| sequence | Sequence | NO. |
| RING domain | TGTCCGGTGTGTCTAGAGCGCCTTGATGCTTCTGCGAAGGTCT | 31 |
| | TGCCTTGCCAGCATACGTTTTGCAAGCGATGTTTGCT | |
| | GGGGATCGTAGGTTCTCGAAATGAACTCAGATGTCCCGAGT | |
| 1^{st} SH₃ | CCATGTGCCAAAGCGTTATACAACTATGAAGGAAAAGAGCCTG | 32 |
| domain | GAGACCTTAAATTCAGCAAAGGCGACATCATCATTTT | |
| | GCGAAGACAAGTGGATGAAAATTGGTACCATGGGGAAGTCAAT | |
| | GGAATCCATGGCTTTTTCCCCACCAACTTTGTGCAGA | |
| | TTATT | |
| 2^{nd} SH₃ | CCTCAGTGCAAAGCACTTTATGACTTTGAAGTGAAAGACAAGG | 33 |
| domain | AAGCAGACAAAGATTGCCTTCCATTTGCAAAGGATGA | |
| | TGTTCTGACTGTGATCCGAAGAGTGGATGAAAACTGGGCTGAA | |
| | GGAATGCTGGCAGACAAAATAGGAATATTTCCAATTT | |
| | CATATGTTGAGTTTAAC | |
| 3^{rd} SH₃ | AGTGTGTATGTTGCTATATATCCATACACTCCTCGGAAAGAGG | 34 |
| domain | ATGAACTAGAGCTGAGAAAAGGGGAGATGTTTTTAGT | |
| | GTTTGAGCGCTGCCAGGATGGCTGGTTCAAAGGGACATCCATG | |
| | CATACCAGCAAGATAGGGGTTTTCCCTGGCAATTATG | |
| | TGGCACCAGTC | |
| 4^{th} _{SH}₃ | GAAAGGCACAGGGTGGTGGTTTCCTATCCTCCTCAGAGTGAGG | 35 |
| domain | CAGAACTTGAACTTAAAGAAGGAGATATTGTGTTTGT | |
| | TCATAAAAAACGAGAGGATGGCTGGTTCAAAGGCACATTACAA | |
| | CGTAATGGGAAAACTGGCCTTTTCCCAGGAAGCTTTG | |
| | TGGAAAACA | |

TABLE 5

Summary of Sequence Identification Numbers

| | Sequence Identi- |
|---|---|
| | fication Number |
| Sequence Information | (SEQ ID NO) |
| Human POSH Coding Sequence | SEQ ID No: 1 |
| Human POSH Amino Acid Sequence | SEQ ID No: 2 |
| Human POSH cDNA Sequence | SEQ ID NO: 3 |
| 5′ cDNA Fragment of Human POSH | SEQ ID No: 4 |
| N-terminus Protein Fragment of | SEQ ID No: 5 |
| Human POSH | |
| 3′ mRNA Fragment of Human POSH | SEQ ID No: 6 |
| C-terminus Protein Fragment of | SEQ ID No: 7 |
| Human POSH | |
| Mouse POSH mRNA Sequence | SEQ ID No: 8 |
| Mouse POSH Protein Sequence | SEQ ID No: 9 |
| Drosophila melanogaster POSH | SEQ ID No: 10 |
| mRNA Sequence | |
| Drosophila melanogaster POSH | SEQ ID No: 11 |
| Protein Sequence | |
| Human POSH RING Domain Amino | SEQ ID No: 26 |
| Acid Sequence | |
| Human POSH 1^{st} SH₃ Domain Amino | SEQ ID No: 27 |
| Acid Sequence | |
| Human POSH 2^{nd} SH₃ Domain Amino | SEQ ID No: 28 |
| Acid Sequence | |
| Human POSH 3^{rd} SH₃ Domain Amino | SEQ ID No: 29 |
| Acid Sequence | |
| Human POSH 4^{th} SH₃ Domain Amino | SEQ ID No: 30 |
| Acid Sequence | |
| Human POSH RING Domain Nucleic | SEQ ID No: 31 |
| Acid Sequence | |
| Human POSH 1^{st} SH₃ Domain Nucleic | SEQ ID No: 32 |
| Acid Sequence | |
| Human POSH 2^{nd} SH₃ Domain Nucleic | SEQ ID No: 33 |
| Acid Sequence | |
| Human POSH 3^{rd} SH₃ Domain Nucleic | SEQ ID No: 34 |
| Acid Sequence | |
| Human POSH 4^{th} SH₃ Domain Nucleic | SEQ ID No: 35 |
| Acid Sequence | |

9. Exemplary Polypeptides

In certain aspects, the methods of the invention also make available isolated and/or purified forms of the subject POSH proteins, which are isolated from, or otherwise substantially free of, other intracellular proteins which might normally be associated with the protein or a particular complex including the protein. In certain embodiments, POSH polypeptides have an amino acid sequence that is at least 60% identical to an amino acid sequence as set forth in any of SEQ ID Nos: 2, 5, 7, 9, 11, 26, 27, 28, 29 and 30. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOS: 2, 5, 7, 9, 11, 26, 27, 28, 29 and 30.

Optionally, a POSH protein of the invention will function in place of a respective endogenous POSH protein, for example by mitigating a respective partial or complete POSH protein loss of function phenotype in a cell. For example, a POSH protein of the invention may be produced in a cell in which endogenous POSH protein has been reduced by RNAi, and the introduced POSH polypeptide will mitigate a phenotype resulting from the RNAi. An exemplary POSH protein loss of function phenotype is a decrease in virus-like particle production in a cell transfected with a viral vector, optionally an HIV vector.

In certain aspects, the methods of the invention also make available isolated and/or purified forms of the subject HERPUD1 proteins, which are isolated from, or otherwise substantially free of, other intracellular proteins which might normally be associated with the protein or a particular complex including the protein. In certain embodiments, HERPUD1 polypeptides have an amino acid sequence that is at least 60% identical to an amino acid sequence as set forth in any of SEQ ID NOS: 47-50. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOS: 47-50.

Optionally, a HERPUD1 protein of the invention will function in place of a respective endogenous HERPUD1 protein, for example by mitigating a respective partial or complete HERPUD1 protein loss of function phenotype in a cell. For example, a HERPUD1 protein of the invention may be produced in a cell in which endogenous HERPUD1 protein has been reduced by RNAi, and the introduced HERPUD1 polypeptide will mitigate a phenotype resulting from the RNAi.

In another aspect, the invention provides polypeptides that are agonists or antagonists of a subject protein, such as agonists or antagonist of POSH or HERPUD1. Variants and fragments of a subject protein, such as a POSH protein, may have a hyperactive or constitutive activity, or, alternatively, act to prevent the subject protein from performing one or more functions. For example, a truncated form lacking one or more domains may have a dominant negative effect.

Another aspect of the invention relates to polypeptides derived from a full-length subject protein, such as fragments derived from a full-length POSH, HERPUD1, and/or Nef protein. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, such as by microinjection assays.

It is also possible to modify the structure of the subject POSH or HERPUD1 proteins for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the POSH or HERPUD1 proteins described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a tlireonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur -containing=cysteine and methionine. (see, for example, Biochemistry, 2nd ed., Ed. by L. Stryer, W. H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a POSH protein can be assessed, e.g., for their ability to bind to another polypeptide, e.g., another POSH protein or another protein involved in viral maturation, cell transformation, or cell proliferation. Polypeptides in which more than one replacement has taken place can readily be tested in the same manner.

This invention further contemplates a method of generating sets of combinatorial mutants of the subject proteins, as well as truncation mutants, and is especially useful for identifying potential variant sequences (e.g., homologs) that are functional in binding to a subject protein, such as POSH or HERPUD1. The purpose of screening such combinatorial libraries is to generate, for example, POSH protein homologs which can act as either agonists or antagonist, or alternatively, which possess novel activities all together. Combinatorially-derived homologs can be generated which have a selective potency relative to a naturally occurring POSH protein. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the POSH protein of interest. Such homologs, and the genes which encode them, can be utilized to alter POSH protein levels by modulating the half-life of the protein. For instance, a short half-life can give rise to more transient biological effects and, when part of an inducible expression system, can allow tighter control of recombinant POSH protein levels within the cell. As above, such proteins, and particularly their recombinant nucleic acid constructs, can be used in gene therapy protocols.

In similar fashion, POSH protein homologs can be generated by the present combinatorial approach to act as antagonists, in that they are able to interfere with the ability of the corresponding wild-type protein to function.

In a representative embodiment of this method, the amino acid sequences for a population of POSH protein homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential POSH protein sequences. For instance, a mixture of synthetic oligonucleotides can be enzymatically ligated into gene sequences such that the degenerate set of potential POSH protein nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential POSH protein sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos: 5,223,409, 5,198,346, and 5,096,815).

Alternatively, other forms of mutagenesis can be utilized to generate a combinatorial library. For example, POSH protein homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of POSH protein.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of POSH protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind, e.g., a POSH protein, is detected in a “panning assay”. For instance, a library of POSH protein variants can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which binds the POSH polypeptide, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject proteins (e.g., POSH, HERPUD1) to generate mimetics, e.g., peptide or non-peptide agents, which are able to mimic binding of the authentic protein to another cellular partner. Such mutagenic techniques as described above, as well as the thioredoxin system, are also particularly useful for mapping the determinants of a subject protein which participate in protein-protein interactions involved in, for example, binding of proteins involved in viral maturation to each other. To illustrate, the critical residues of a POSH protein which are involved in molecular recognition of a substrate protein can be determined and used to generate POSH protein-derived peptidomimetics which bind to the substrate protein, and by inhibiting POSH protein binding, act to inhibit its biological activity. By employing, for example, scanning mutagenesis to map the amino acid residues of a POSH protein which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

The following table provides the sequences of the RING domain and the various SH3 domains.

TABLE 6

Amino Acid Sequences and related SEQ ID NOs for domains
in human POSH

| Name of | | |
|---|---|---|
| the | | SEQ ID |
| sequence | Sequence | NO. |
| RING | CPVCLERLDASAKVLPCQHTFCKRCLLGIVGSRNELRCPEC | 26 |
| domain | | |
| 1^{st} SH₃ | PCAKALYNYEGKEPGDLKFSKGDIIILRRQVDENWYHGEVNGIHGF | 27 |
| domain | FPTNFVQIIK | |
| 2^{nd} SH₃ | PQCKALYDFEVKDKEADKDCLPFAKDDVLTVIRRVDENWAEGMLAD | 28 |
| domain | KIGIFPISYVEFNS | |
| 3^{rd} SH₃ | SVYVAIYPYTPRKEDELELRKGEMFLVFERCQDGWFKGTSMHTSKI | 29 |
| domain | GVFPGNYVAPVT | |
| 4^{th} SH₃ | ERHRVVVSYPPQSEAELELKEGDIVFVHKKREDGWFKGTLQRNGKT | 30 |
| domain | GLFPGSFVENI | |

10. Antibodies and Uses Thereof

Another aspect of the invention pertains to an antibody specifically reactive with a POSH or HERPUD1 protein. For example, by using immunogens derived from a POSH or HERPUD1 protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a POSH polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a POSH or HERPUD1 protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a POSH or HERPUD1 protein of a mammal.

The term antibody as used herein is intended to include fragments thereof which are also specifically reactive with one of the subject POSH or HERPUD1 proteins. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antibodies of the present invention are further intended to include bispecific, single-chain, and chimeric and humanized molecules having affinity for a POSH or HERPUD1 protein conferred by at least one CDR region of the antibody. In preferred embodiments, the antibody further comprises a label attached thereto and able to be detected, (e.g., the label can be a radioisotope, fluorescent compound, enzyme or enzyme co-factor).

Anti-POSH or anti-HERPUD1 protein antibodies can be used, e.g., to monitor POSH or HERPUD1 protein levels, respectively, in an individual and in certain instances, particularly the presence of POSH protein at the plasma membrane for determining whether or not said patient is infected with a virus such as an RNA virus, a retroid virus, and an envelope virus, or allowing determination of the efficacy of a given treatment regimen for an individual afflicted with such a disorder. In addition, POSH protein polypeptides are expected to localize, occasionally, to the released viral particle. Viral particles may be collected and assayed for the presence of a POSH protein. The level of POSH and/or HERPUD1 protein may be measured in a variety of sample types such as, for example, cells and/or in bodily fluid, such as in blood samples.

11. Drug Screening Assays

In certain aspects, the present invention also provides assays for identifying therapeutic agents that interfere with the trafficking and/or assembly of protein assemblies in the trans Golgi network, including proteins associated with viral-related disorders, such as AIDS. In certain embodiments, agents of the invention are antiviral agents, optionally interfering with viral maturation, and preferably where the virus is a retroid virus, an RNA virus and an envelope virus.

In certain preferred embodiments, an antiviral agent interferes with an activity of a HERPUD1 protein. In certain embodiments, an antiviral agent interferes with the expression of a HERPUD1 polypeptide, for example, by decreasing the level of HERPUD1 polypeptide in a cell. In certain preferred embodiments, an antiviral agent interferes with the interaction between HERPUD1 and a HERPUD1-AP polypeptide, for example an antiviral agent may disrupt or render irreversible the interaction between a HERPUD1 protein and a HERPUD1-AP, such as, for example, a HERPUD1-AP selected from among a Nef polypeptide and a POSH polypeptide. In additional embodiments, an antiviral agent interferes with the ubiquitination of a HERPUD1 polypeptide. For example, in one embodiment, an antiviral agent interferes with the ubiquitination of HERPUD1 by a POSH polypeptide.

In certain embodiments, the invention provides assays to identify, optimize or otherwise assess agents that increase or decrease a ubiquitin-related activity of a POSH protein. Ubiquitin-related activities of POSH proteins may include, for example, the self-ubiquitination activity of a POSH protein, generally involving the transfer of ubiquitin from an E2 enzyme to the POSH protein, and the ubiquitination of a target protein, generally involving the transfer of a ubiquitin from a POSH protein to the target protein, such as for example, to a HERPUD1 protein. In certain embodiments, a POSH protein activity is mediated, at least in part, by a RING domain.

In certain embodiments, an assay comprises forming a mixture comprising a POSH protein that is an E3 polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the POSH E3 polypeptide. One or more of a variety of parameters may be detected, such as POSH protein-ubiquitin conjugates, E2-ubiquitin thioesters, free ubiquitin and target polypeptide-ubiquitin complexes. The term “detect” is used herein to include a determination of the presence or absence of the subject of detection (e.g., POSH-ubiqutin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the POSH protein-ubiquitin conjugate.

In certain embodiments, an assay comprises forming a mixture comprising a POSH protein, a target polypeptide (e.g., HERPUD1) and a source of ubiquitin (which may be the POSH polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as POSH protein-ubiquitin conjugates and target polypeptide-ubiquitin conjugates. In a preferred embodiment, an assay comprises detecting the target polypeptide-ubiquitin conjugate. In another preferred embodiment, an assay comprises detecting the POSH protein-ubiquitin conjugate.

An assay described above may be used in a screening assay to identify agents that modulate a ubiquitin-related activity of a POSH polypeptide. A screening assay will generally involve adding a test agent to one of the above assays, or any other assay designed to assess a ubiquitin-related activity of a POSH protein. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g., the POSH protein), followed by removing the test agent and addition of other components to complete the assay. In this manner, the effects of the agent solely on the POSH protein may be assessed. In certain preferred embodiments, a screening assay for an antiviral agent employs a target polypeptide comprising an L domain, and preferably an HIV L domain.

In certain embodiments, an assay is performed in a high-throughput format. For example, one of the components of a mixture may be affixed to a solid substrate and one or more of the other components is labeled. For example, the POSH protein may be affixed to a surface, such as a 96-well plate, and the ubiquitin is in solution and labeled. An E2 and E1 are also in solution, and the POSH protein-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., POSH protein or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the POSH protein or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As described above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a POSH protein.

In an alternative embodiment, a POSH protein, E2 or target polypeptide is bound to a bead, optionally with the assistance of a tag. Following ligation, the beads may be separated from the unbound ubiquitin and the bound ubiquitin measured. In a preferred embodiment, POSH protein is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, POSH protein is combined at a final concentration of from 1 ng to 500 ng per 100 microliter reaction solution.

Generally, an assay mixture is prepared so as to favor ubiquitin ligase activity and/or ubiquitination acitivty. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40 degrees C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a POSH protein ubiquitin-related activity may be used to inhibit POSH protein function in vivo, while a test agent that increases a POSH protein ubiquitin-related activity may be used to stimulate POSH protein function in vivo. A test agent may be modified for use in vivo, e.g., by addition of a hydrophobic moiety, such as an ester.

Certain embodiments of the invention relate to assays for identifying agents that bind to a HERPUD1 protein or to a POSH protein, optionally a particular domain of POSH protein such as an SH3 or RING domain. In certain embodiments, a POSH protein is a POSH polypeptide that is a polypeptide comprising the fourth SH3 domain of HPOSH (SEQ ID NO: 30). A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject POSH polypeptides with a POSH-AP. In another embodiment, the assay detects agents which modulate the intrinsic biological activity of a POSH protein or POSH protein complex, such as an enzymatic activity, binding to other cellular components, cellular compartmentalization, and the like. In another embodiment, an assay detects agents which inhibit interaction of one or more subject HERPUD1 polypeptides with a HERPUD1-AP. In another embodiment, the assay detects agents which modulate the intrinsic biological activity of a HERPUD1 protein or HERPUD1 protein complex, such as an enzymatic activity, binding to other cellular components, cellular compartmentalization, and the like.

In one aspect, the invention provides methods and compositions for the identification of compositions that interfere with the function of a POSH or HERPUD1 protein. Given the role of POSH and HERPUD1 proteins in viral production, compositions that perturb the formation or stability of the protein-protein interactions between POSH and/or HERPUD1 proteins and the proteins that they interact with are candidate pharmaceuticals for the treatment of viral infections.

While not wishing to be bound to mechanism, it is postulated that POSH proteins promote the assembly of protein complexes that are important in release of virions and other biological processes. Complexes of the invention may include a combination of a POSH polypeptide and one or more of the following POSH-APs: a Nef polypeptide, a PACS-1 polypeptide, an HLA-A polypeptide, and an HLA-B polypeptide, a POSH polypeptide and POSH-AP polypeptide such as another POSH polypeptide (as in the case of a POSH dimer, a heterodimer of two different POSH, homomultimers and heteromultimers); Cbl-b; an MSTP028; a HERPUD1; a GTPase (eg. Rac, Rac1, Rho, Ras); an E2 enzyme; ubiquitin; PAK1, PAK2, PAK family, Vav, Cdc42, PI3K (e.g., p85 or p110), a Gag, particularly an HIV Gag (e.g., p160); Vpu; as well as, in certain embodiments, proteins known to be associated with clathrin-coated vesicles and or proteins involved in the protein sorting pathway.

The type of complex formed by a POSH polypeptide will depend upon the domains present in the protein. While not intended to be limiting, exemplary domains of potential interacting proteins are provided below. A RING domain is expected to interact with cullins, E2 enzymes, AP-1, AP-2, and/or a substrate for ubiquitination (e.g., in some instances, a protein comprising a Gag L domain or a Gag polypeptide such as Gag-Pol, such as HIV p160). An SH3 domain may interact with Gag L domains and other proteins having the sequence motif P(T/S)AP, RXXP(T/S)AP, PXXDY, PXXP, PPXY, RXXPXXP, or PXXPXR such as, for example, an HIV Gag sequence such as RQGPKEPFR, PFRDY, PTAP and RPEPTAP.

In a preferred assay for an antiviral or antiapoptotic agent, the test agent is assessed for its ability to disrupt or inhibit the formation of a complex of a POSH polypeptide and a Rac polypeptide, particularly a human Rac polypeptide, such as Rac1.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a POSH protein-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to a POSH or HERPUD1 protein. Such binding assays may also identify agents that act by disrupting the interaction between a POSH or HERPUD1 protein and an interacting protein, or the binding of a POSH or HERPUD1 protein or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted POSH protein complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in POSH protein complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure POSH protein complex assembly and/or disassembly.

Assaying POSH or HERPUD1 protein complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of a POSH or HERPUD1 protein complex. In an exemplary binding assay, the compound of interest is contacted with a mixture comprising a POSH or HERPUD1 protein and at least one interacting polypeptide. Detection and quantification of POSH or HERPUD1 protein complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between POSH or HERPUD1 proteins and a substrate polypeptide may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST- POSH protein fusion proteins can be adsorbed onto glutathione sepharose beads (Sigma Chemical, St. Louis, Mo.) or glutathione derivatized microtitre plates, which are then combined with a potential interacting protein, e.g., an 35S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a POSH or HERPUD1 protein may be identified by using an immobilized POSH or HERPUD1 protein. In an illustrative embodiment, a fusion protein can be provided which adds a domain that permits the protein to be bound to an insoluble matrix. For example, GST-POSH protein fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the POSH or HERPUD1 protein and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a POSH protein of sufficient length to bind to a potential interacting protein. The second hybrid protein encodes a transcriptional activation domain fused in frame to a gene encoding a “fish” protein, e.g., a potential interacting protein of sufficient length to interact with the POSH protein portion of the bait fusion protein. If the bait and fish proteins are able to interact, e.g., form a POSH protein complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S. cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector. Interaction trap assays may also be performed in mammalian and bacterial cell types.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a POSH protein sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, lcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative or other mutants of a POSH protein can be used.

Continuing with the illustrated example, the POSH protein-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a POSH protein complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations including a POSH or HERPUD1 protein and one or more interacting polypeptides.

In still further embodiments of the present assay, a POSH or HERPUD1 protein complex is generated in whole cells, taking advantage of cell culture techniques to support the subject assay. For example, as described below, the POSH or HERPUD1 protein complex can be constituted in a eukaryotic cell culture system, including mammalian and yeast cells. Often it will be desirable to express one or more viral proteins (e.g., Nef or Gag or Env or Vpu) in such a cell along with a subject POSH or HERPUD1 protein. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the POSH or HERPUD1 protein complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a POSH or HERPUD1 protein activity. In certain embodiments a POSH or HERPUD1 protein activity is represented by production of virus like particles. As demonstrated herein, an agent that disrupts POSH protein activity can cause a decrease in the production of viral like particles. Other bioassays for POSH protein activities may include apoptosis assays (e.g., cell survival assays, apoptosis reporter gene assays, etc.) and NF-kB nuclear localization assays (see e.g., Tapon et al. (1998) EMBO J. 17: 1395-1404).

In certain embodiments, POSH protein activities may include, without limitation, complex formation, ubiquitination and membrane fusion events (e.g., release of viral buds or fusion of vesicles). POSH or HERPUD1 protein complex formation may be assessed by immunoprecipitation and analysis of co-immunoprecipiated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, the POSH or HERPUD1 polypeptide and the interacting protein of interest are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria Victoria. Variants may be brighter, dimmer, or have different excitation and/or emission spectra. Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (eg. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808; Levine et al. (1982) Comp. Biochem. Physiol., 72B:77-85; Tersikh et al. (2000) Science 290: 1585-88.

Other exemplary fluorescent moieties well known in the art include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri (Baldwin et al. (1990) Biochemistry 29:5509-15), peridinin-chlorophyll a binding protein from the dinoflagellate Symbiodinium sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

FRET-based assays may be used in cell-based assays and in cell-free assays. FRET-based assays are amenable to high-throughput screening methods including Fluorescence Activated Cell Sorting and fluorescent scanning of microtiter arrays.

In a further embodiment, transcript levels may be measured in cells having higher or lower levels of POSH or HERPUD1 protein activity in order to identify genes that are regulated by POSH or HERPUD1 proteins, respectively. Promoter regions for such genes (or larger portions of such genes) may be operatively linked to a reporter gene and used in a reporter gene-based assay to detect agents that enhance or diminish POSH or HERPUD1 protein-regulated gene expression. Transcript levels may be determined in any way known in the art, such as, for example, Northern blotting, RT-PCR, microarray, etc. Increased POSH or HERPUD1 protein activity may be achieved, for example, by introducing a strong POSH or HERPUD1 protein expression vector, respectively. Decreased POSH or HERPUD1 protein activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the invention provides methods for identifying targets for therapeutic intervention. A polypeptide that interacts with a POSH or HERPUD1 protein or participates in a POSH or HERPUD1 protein-mediated process (such as viral maturation) may be used to identify candidate therapeutics. Such targets may be identified by identifying proteins that associated with POSH or HERPUD1 proteins by, for example, immunoprecipitation with an anti-POSH or anti-HERPUD1 protein antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti- microbial agents, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In certain embodiments, a test agent may be assessed for its ability to perturb the localization of a POSH or HERPUD1 protein.

12. Effective Dose

Toxicity and therapeutic efficacy of such compounds can be determined by standard pharmaceutical procedures in cell cultures or experimental animals, e.g., for determining The Ld₅ (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

13. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the invention comprises an RNAi mixed with a delivery system, such as a liposome system, and optionally including an acceptable excipient. In a preferred embodiment, the composition is formulated for topical administration for, e.g., herpes virus infections.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

### Exemplification

The application now being generally described, it will be more readily understood by reference to the following examples, which are included merely for purposes of illustration of certain aspects and embodiments of the present invention, and are not intended to limit the invention.

### EXAMPLES

### Example 1

### Role of POSH in Virus-like Particle (VLP) Budding

1. Objective:

Use RNAi to inhibit POSH gene expression and compare the efficiency of viral budding and GAG expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods

i. Transfections according to manufacturer's protocol and as described in procedure.

ii. Protein determined by Bradford assay.

iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life | 11668-019 | 1112496 |
| (LF2000) | Technologies | | |
| OptiMEM | Life | 31985-047 | 3063119 |
| | Technologies | | |
| RNAi Lamin A/C | Self | 13 | |
| RNAi TSG101 688 | Self | 65 | |
| RNAi Posh 524 | Self | 81 | |
| plenvl1 PTAP | Self | 148 | |
| plenvl1 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life | NP0321 | 1081371 |
| Glycine SDS-PAGE gel | Technologies | | |
| Nitrocellulose | Schleicher & | 401353 | BA-83 |
| membrane | Schuell | | |
| NuPAGE 20× transfer | Life | NP0006-1 | 224365 |
| buffer | Technologies | | |
| 0.45 μm filter | Schleicher & | 10462100 | CS1018-1 |
| | Schuell | | |

c. Solutions

| | Compound | Concentration | |
|---|---|---|---|
| Lysis Buffer | Tris-HCl pH 7.6 | 50 | mM |
| | MgCl₂ | 15 | mM |
| | NaCl | 150 | mM |
| | Glycerol | 10% | |
| | EDTA | 1 | mM |
| | EGTA | 1 | mM |
| | ASB-14 (add immediately | 1% | |
| | before use) | | |
| 6× Sample | Tris-HCl, pH = 6.8 | 1 | M |
| Buffer | Glycerol | 30% | |
| | SDS | 10% | |
| | DTT | 9.3% | |
| | Bromophenol Blue | 0.012% | |
| TBS-T | Tris pH = 7.6 | 20 | mM |
| | NaCl | 137 | mM |
| | Tween-20 | 0.1% | |

4. Procedure

a. Schedule

Day

| 1 | 2 | 3 | 4 | 5 |
|---|---|---|---|---|
| Plate | Transfection | Passage | Transfection II | Extract RNA |
| cells | I | cells | (RNAi and | for RT-PCR |
| | (RNAi only) | (1:3) | pNlenv) | (post |
| | | | (12:00, PM) | transfection) |
| | | | Extract RNA for | Harvest VLPs |
| | | | RT-PCR | and cells |
| | | | (pre-transfection) | |

b. Day 1

Plate HeLa SS-6 cells in 6-well plates (35 mm wells) at concentration of 5×10⁵ cells/well.
c. Day2

2 hours before transfection replace growth medium with 2 ml growth medium without antibiotics.

Transfecfion I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:
Prepare LF2000 mix: 250 μl OptiMEM+5 μl LF2000 for each reaction. Mix by inversion, 5 times. Incubate 5 minutes at room temperature.
Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.
Add 500 μl transfection mixture to cells dropwise and mix by rocking side to side.

Incubate overnight.
d. Day3
Split 1:3 after 24 hours. (Plate 4 wells for each reaction, except reaction 2 which is plated into 3 wells.)
e. Day4

2 hours pre-transfection replace medium with DMEM growth medium without antibiotics.

Transfection II

| | | | | | B | | |
|---|---|---|---|---|---|---|---|
| | | | | A | RNAi | | |
| | | | | Plasmid | [20 μM] for | C | D |
| RNAi | | | Reaction | for 2.4 μg | 10 nM | OPtiMEM | LF2000 mix |
| name | TAGDA# | Plasmid | # | (μl) | (μl) | (μl) | (μl) |
| Lamin A/C | 13 | PTAP | 3 | 3.4 | 3.75 | 750 | 750 |
| Lamin A/C | 13 | ATAP | 3 | 2.5 | 3.75 | 750 | 750 |
| TSG101 688 | 65 | PTAP | 3 | 3.4 | 3.75 | 750 | 750 |
| Posh 524 | 81 | PTAP | 3 | 3.4 | 3.75 | 750 | 750 |

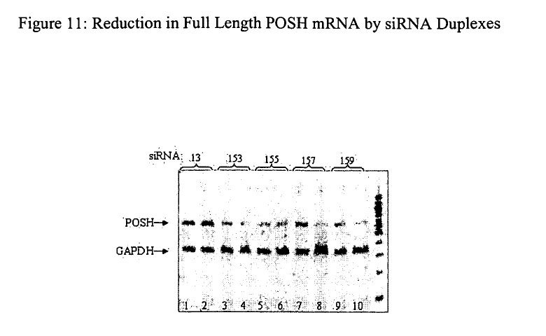

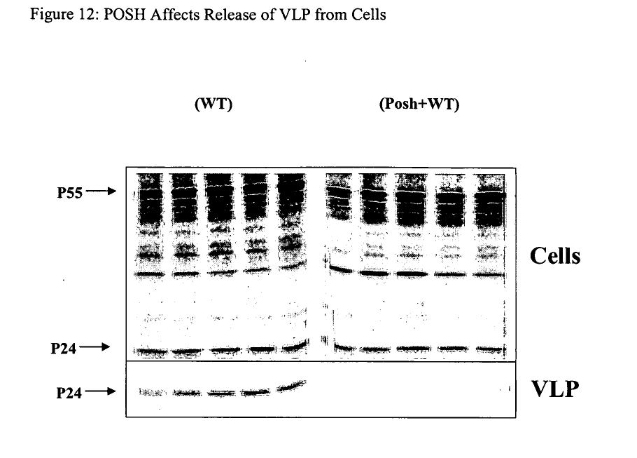

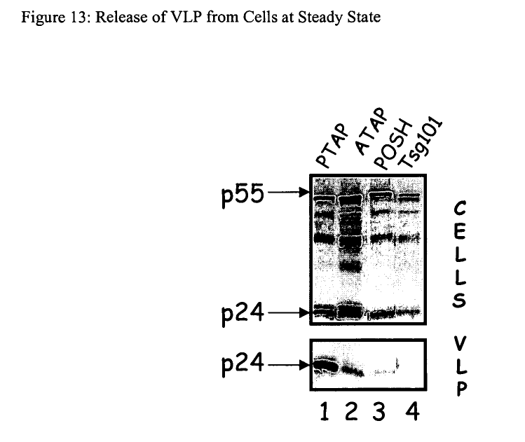

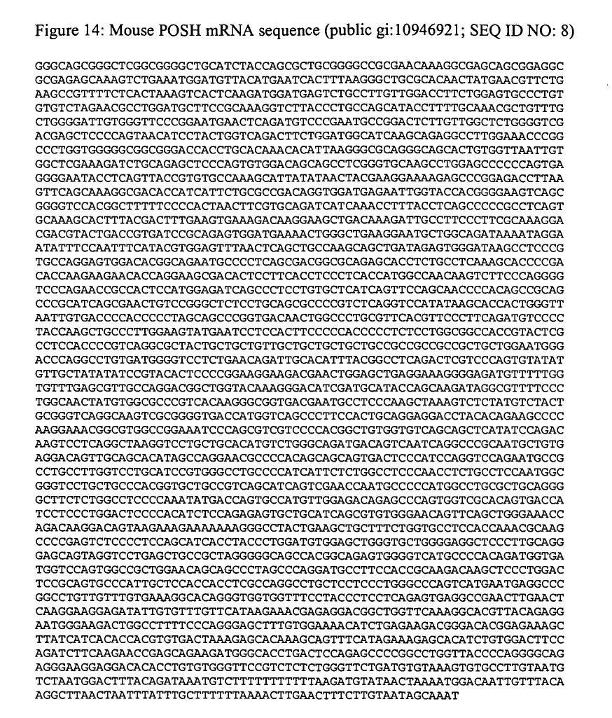

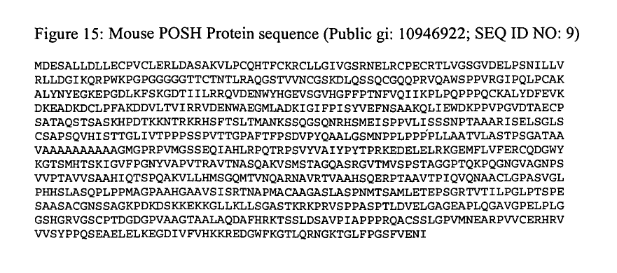

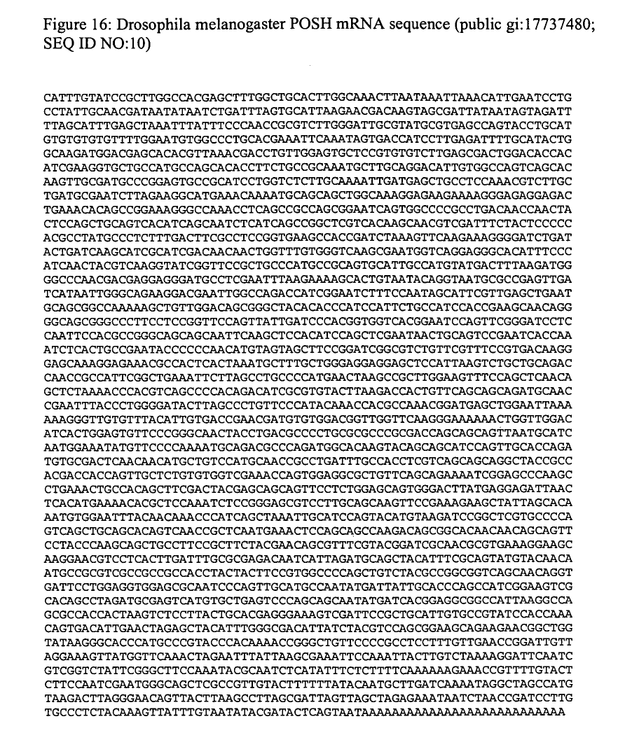

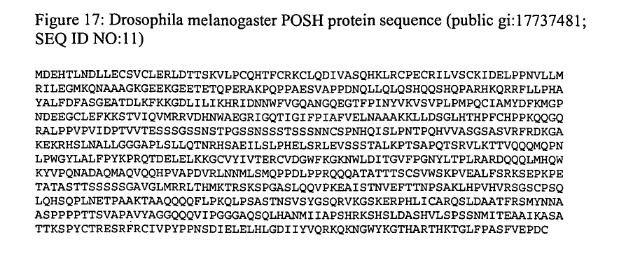

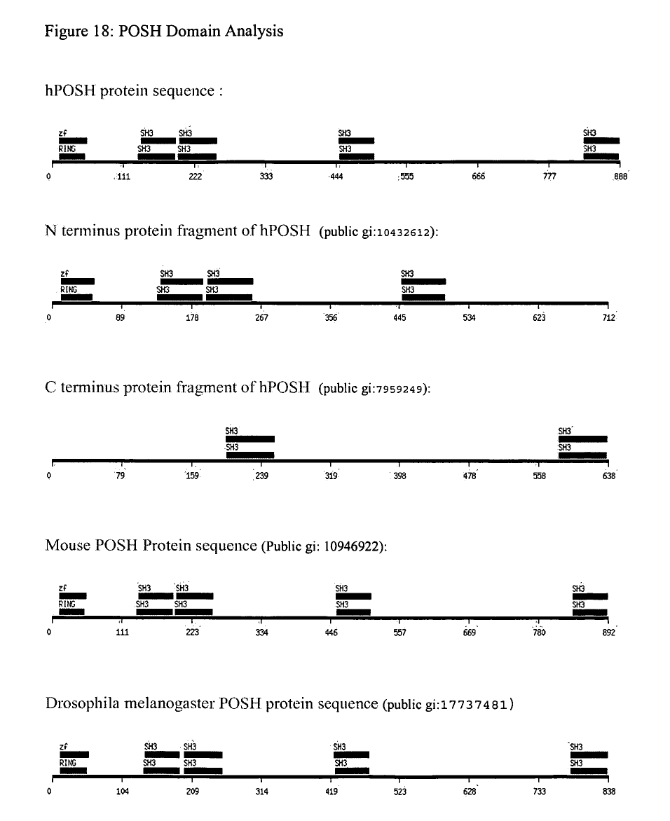

Prepare LF2000 mix: 250 μl OptiMEM+5 μI LF2000 for each reaction. Mix by inversion, 5 times. Incubate 5 minutes at room temperature.
Prepare RNA+DNA diluted in OptiMEM (Transfection II, A+B+C) Add LF2000 mix (Transfection II, D) to diluted RNA+DNA dropwise, mix by gentle vortex, and incubate 1 h while protected from light with aluminum foil.
Add LF2000 and DNA+RNA to cells, 500 μl/well, mix by gentle rocking and incubate overnight.
f. Day5
Collect samples for VLP assay (approximately 24 hours post-transfection) by the following procedure (cells from one well from each sample is taken for RNA assay, by RT-PCR).
g. Cell Extracts
   i. Pellet floating cells by centrifugation (5min, 3000 rpm at 4° C), save supernatant (continue with supernatant immediately to step h), scrape remaining cells in the medium which remains in the well, add to the corresponding floating cell pellet and centrifuge for 5 minutes, 1800 rpm at 4° C.
   ii. Wash cell pellet twice with ice-cold PBS.
   iii. Resuspend cell pellet in 100 μl lysis buffer and incubate 20 minutes on ice.
   iv. Centrifuge at 14,000 rpm for 15 min. Transfer supernatant to a clean tube. This is the cell extract.
   v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.
h. Purification of VLPs from cell media
   i. Filter the supernatant from step g through a 0.45 m filter.
   ii. Centrifuge supernatant at 14,000 rpm at 4° C. for at least 2 h.
   iii. Aspirate supernatant carefully.
   iv. Re-suspend VLP pellet in hot (100° C. warmed for 10 min at least) 1× sample buffer.
   v. Boil samples for 10 minutes, 100° C.
i. Western Blot analysis
   i. Run all samples from stages A and B on Tris-Glycine SDS-PAGE 10% (120V for 1.5 h).
   ii. Transfer samples to nitrocellulose membrane (65V for 1.5 h).
   iii. Stain membrane with ponceau S solution.
   iv. Block with 10% low fat milk in TBS-T for 1 h.
   v. Incubate with anti p24 rabbit 1:500 in TBS-T o/n.
   vi. Wash 3 times with TBS-T for 7 min each wash.
   vii. Incubate with secondary antibody anti rabbit cy5 1:500 for 30 min.
   viii. Wash five times for 10 min in TBS-T.
   ix. View in Typhoon gel imaging system (Molecular Dynamics/APBiotech) for fluorescence signal.
      Results are Shown in FIGS. 11-13.

### Example 2

### Exemplary POSH RT-PCR Primers and siRNA Duplexes

RT-PCR Primers

| | | |
|---|---|---|
| Name | Position | Sequence |

| | | | | |
|---|---|---|---|---|
| Sense primer | POSH = 271 | 271 | 5′ CTTGCCTTGCCAGCATAC 3′ | (SEQ ID NO: 12) |
| Anti-sense | POSH = 926c | 926C | 5′ CTGCCAGCATTCCTTCAG 3′ | (SEQ ID NO: 13) |
| primer | | | | |

siRNA Duplexes:

| | | |
|---|---|---|
| siRNA No: | 153 | |
| siRNA Name: | POSH-230 | |
| Position in | 426-446 | |
| mRNA | | |
| Target | 5′ | SEQ ID NO: 14 |
| sequence: | AACAGAGGCCTTGGAAACCTG 3′ | |
| siRNA sense | 5′ | SEQ ID NO: 15 |
| strand: | dTdTCAGAGGCCUUGGAAACCUG | |
| | 3′ | |
| siRNA anti- | 5′ | |
| sense strand: | dTdTCAGGUUUCCAAGGCCUCUG | SEQ ID NO: 16 |
| | 3′ | |
| siRNA No: | 155 | |
| siRNA Name: | POSH-442 | |
| Position in | 638-658 | |
| mRNA | | |
| Target | 5′ | |
| sequence: | AAAGAGCCTGGAGACCTTAAA 3′ | SEQ ID NO: 17 |
| siRNA sense | 5′ | SEQ ID NO: 18 |
| strand: | ddTdTAGAGCCUGGAGACCUUAAA | |
| | 3′ | |
| siRNA anti- | 5′ | SEQ ID NO: 19 |
| sense strand: | ddTdTUUUAAGGUCUCCAGGCUCU | |
| | 3′ | |
| siRNA No: | 157 | |
| siRNA Name: | POSH-U111 | |
| Position in | 2973-2993 | |
| mRNA | | |
| Target | 5′ | SEQ ID NO: 20 |
| sequence: | AAGGATTGGTATGTGACTCTG 3′ | |
| siRNA sense | 5′ | SEQ ID NO: 21 |
| strand: | dTdTGGAUUGGUAUGUGACUCUG | |
| | 3′ | |
| siRNA anti- | 5′ | SEQ ID NO: 22 |
| sense strand: | dTdTCAGAGUCACAUACCAAUCC | |
| | 3′ | |
| siRNA No: | 159 | |
| siRNA Name: | POSH-U410 | |
| Position in | 3272-3292 | |
| mRNA | | |
| Target | 5′ | SEQ ID NO: 23 |
| sequence: | AAGCTGGATTATCTCCTGTTG 3′ | |
| siRNA sense | 5′ | SEQ ID NO: 24 |
| strand: | ddTdTGCUGGAUUAUCUCCUGUUG | |
| | 3′ | |
| siRNA anti- | 5′ | SEQ ID NO: 25 |
| sense strand: | ddTdTCAACAGGAGAUAAUCCAGC | |
| | 3′ | |
| siRNA No: | 187 | |
| siRNA Name: | POSH-control | |
| Position in | None. Reverse to #153 | |
| mRNA: | | |
| Target | 5′ | SEQ ID NO: 36 |
| sequence: | AAGTCCAAAGGTTCCGGAGAC 3′ | |

3. Knock-Down of hPOSH Entraps HIV Virus Particles in Intracellular Vesicles

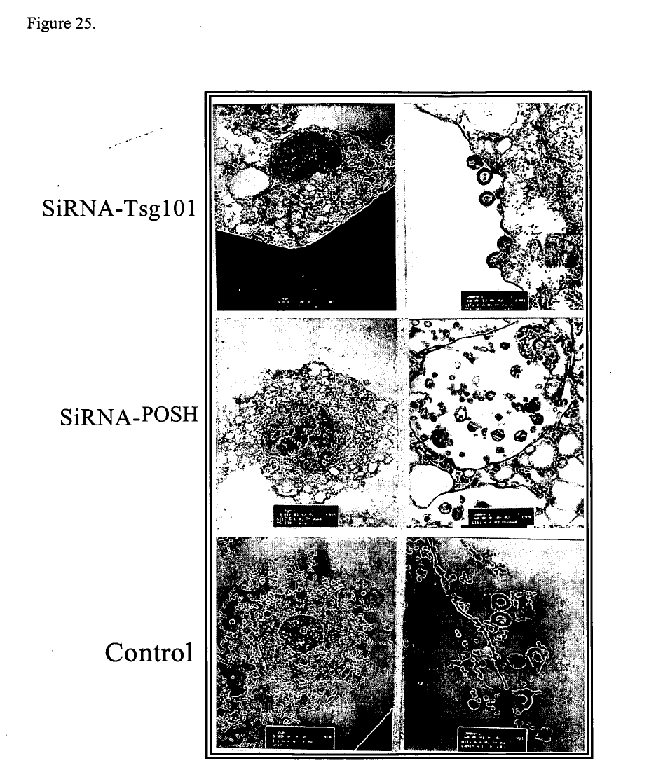

HIV virus release was analyzed by electron microscopy following siRNA and full-length HIV plasmid (missing the envelope coding region) transfection. Mature viruses were secreted by cells transfected with HIV plasmid and non-relevant siRNA (control, lower panel). Knockdown of Tsg101 protein resulted in a budding defect, the viruses that were released had an immature phenotype (upper panel). Knockdown of hPOSH levels resulted in accumulation of viruses inside the cell in intracellular vesicles (middle panel). Results, shown in FIG. 25, indicate that inhibiting hPOSH entraps HIV virus particles in intracellular vesicles. As accumulation of HIV virus particles in the cells accelerate cell death, inhibition of HPOSH therefore destroys HIV reservoir by killing cells infected with HIV.

### Example 4

### In-Vitro Assay of Human POSH Self-Ubiquitination

Recombinant hPOSH was incubated with ATP in the presence of E1, E2 and ubiquitin as indicated in each lane. Following incubation at 37° C. for 30 minutes, reactions were terminated by addition of SDS-PAGE sample buffer. The samples were subsequently resolved on a 10% polyacrylamide gel. The separated samples were then transferred to nitrocellulose and subjected to immunoblot analysis with an anti ubiquitin polyclonal antibody. The position of migration of molecular weight markers is indicated on the right.
Poly-Ub: Ub-hPOSH conjugates, detected as high molecular weight adducts only in reactions containing E1, E2 and ubiquitin. hPOSH-176 and hPOSH-178 are a short and a longer derivatives (respectively) of bacterially expressed hPOSH; C, control E3.
   Preliminary Steps in a High-Throughput Screen
   Materials
1. E1 recombinant from bacculovirus
2. E2 Ubch5c from bacteria
3. Ubiquitin
4. POSH #178 (1-361) GST fusion-purified but degraded
5. POSH # 176 (1-269) GST fusion-purified but degraded
6. hsHRD1 soluble ring containing region
5. Bufferx12 (Tris 7.6 40 mM, DTT 1 mM, MgCl₂ 5 mM, ATP 2 uM)

6. Dilution buffer (Tris 7.6 40 mM, DTT 1 mM, ovalbumin 1 ug/ul) protocol

| | 0.1 ug/ul | 0.5 ug/ul | 5 ug/ul | 0.4 ug/ul | 2.5 ug/u/ | 0.8 ug/ul | |
|---|---|---|---|---|---|---|---|
| | E1 | E2 | Ub | 176 | 178 | Hrd1 | Bx12 |
| −E1 (E2 + 176) | — | 0.5 | 0.5 | 1 | — | — | 10 |
| −E2 (E1 + 176) | 1 | — | 0.5 | 1 | — | — | 9.5 |
| −ub (E1 + E2 + 176) | 1 | 0.5 | — | 1 | — | — | 9.5 |
| E1 + E2 + 176 + Ub | 1 | 0.5 | 0.5 | 1 | | — | 9 |
| −E1 (E2 + 178) | — | 0.5 | 0.5 | — | 1 | — | 10 |
| −E2 (E1 + 178) | 1 | — | 0.5 | — | 1 | — | 9.5 |
| −ub (E1 + E2 + 178) | 1 | 0.5 | — | — | 1 | — | 9.5 |
| E1 + E2 + 178 + Ub | 1 | 0.5 | 0.5 | — | 1 | —1 | 9 |
| Hrd1, E1 + E2 + Ub | 1 | 0.5 | 0.5 | — | — | 1 | 8.5 |
| | | | | | | | * |

1. Incubate for 30 minutes at 37° C.
2. Run 12% SDS PAGE gel and transfer to nitrocellulose membrane
3. Incubate with anti-Ubiquitin antibody.

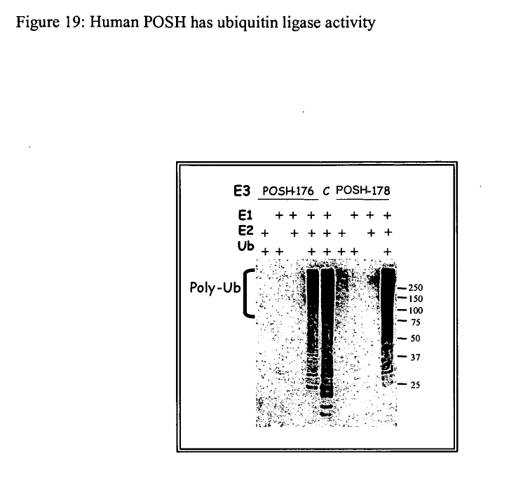

Results, shown in FIG. 19, demonstrate that human POSH has ubiquitin ligase activity.

### Example 5

### Co-Immunoprecipitation of hPOSH with mvc-Tagged Activated (V12) and Dominant-Negative (N17) Rac1

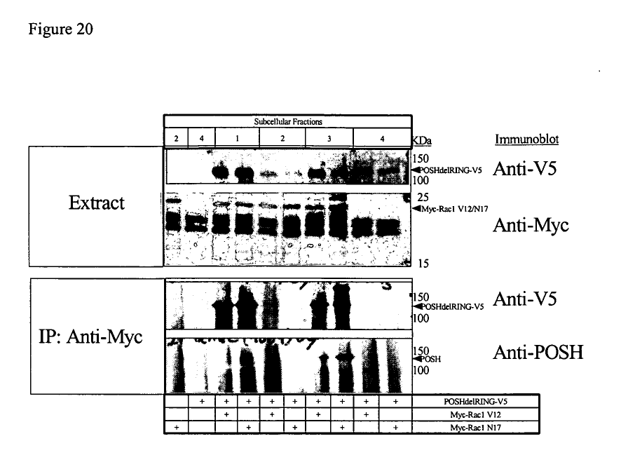

HeLa cells were transfected with combinations of myc-Rac1 V12 or N17 and hPOSHdelRING-V5. 24 hours after transfection (efficiency 80% as measured by GFP) cells were collected, washed with PBS, and swollen in hypotonic lysis buffer (10 mM HEPES pH=7.9, 15 mM KCl, 0.1 mM EDTA, 2 mM MgCl2, 1 mM DTT, and protease inhibitors). Cells were lysed by 10 strokes with dounce homogenizer and centrifuged 3000×g for 10 minutes to give supernatant (Fraction 1) and nucleii. Nucleii were washed with Fraction 2 buffer (0.2% NP-40, 10 mM HEPES pH=7.9, 40 mM KCl, 5% glycerol) to remove peripheral proteins. Nucleii were spun-down and supernatant collected (Fraction 2). Nuclear proteins were eluted in Fraction 3 buffer (20 mM HEPES pH=7.9, 0.42 M KCl, 25% glycerol, 0.1 mM EDTA, 2 mM MgCl₂, 1 mM DTT) by rotating 30 minutes in cold. Insoluble proteins were spun-down 14000×g and solubilized in Fraction 4 buffer (1% Fos-Choline 14, 50 mM HEPES pH=7.9, 150 mM NaCl, 10% glycerol, 1 mM EDTA, 1.5 mM MgCl₂, 2 mM DTT). Half of the total extract was pre-cleared against Protein A sepharose for 1.5 hours and used for IP with 1 μg anti-myc (9E10, Roche 1-667-149) and Protein A sepharose for 2 hours. Immune complexes were washed extensively, and eluted in SDS-PAGE sample buffer. Gels were run, and proteins electro-transferred to nitrocellulose for immunoblot as in FIG. 20. Endogenous POSH and transfected hPOSHdelRING-V5 are precipitated as a complex with Myc-Rac1 V12/N17. Results, shown in FIG. 20, demonstrate that POSH co-immunoprecipitates with Rac1.

### Example 6

### Effect of hPOSH on Gag-EGFP Intracellular Distribution

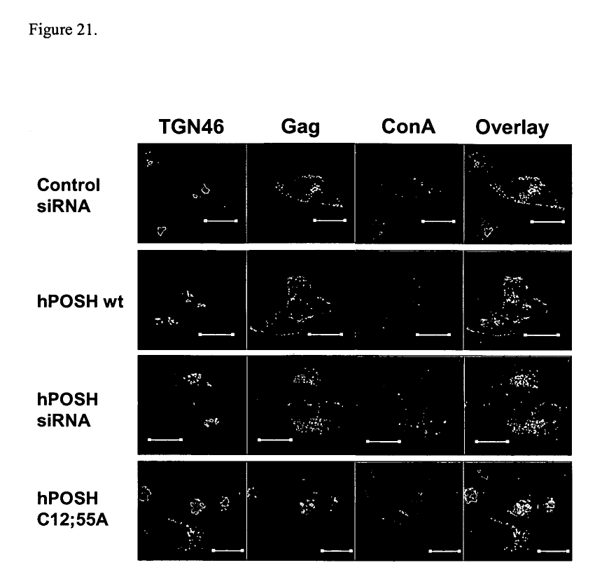

HeLa SS6 were transfected with Gag-EGFP, 24 hours after an initial transfection with either hPOSH-specific or scrambled siRNA (control) (100 nM) or with plasmids encoding either wild type hPOSH or hPOSH C(12,55)A. Fixation and staining was preformed 5 hours after Gag-EGFP transfection. Cells were fixed, stained with Alexa fluor 647-conjugated Concanavalin A (ConA) (Molecular Probes), permeabilized and then stained with sheep anti-human TGN46. After the primary antibody incubation cells were incubated with Rhodamin-conjugated goat anti-sheep. Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown. See FIG. 21.

### Example 7

### POSH-Regulated Intracellular Transport of Myristoylated Proteins

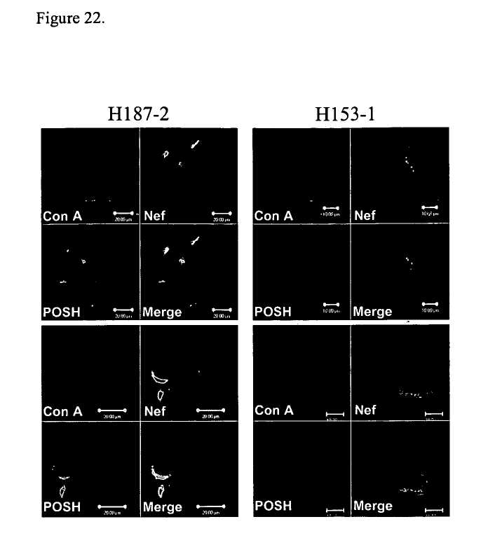

The localization of myristoylated proteins, Gag (see FIG. 21), HIV-1 Nef, Src and Rapsyn, in cells depleted of hPOSH were analyzed by immunofluorescence. In control cells, HIV-1 Nef was found in a perinuclear region co-localized with hPOSH, indicative of a TGN localization (FIG. 22). When hPOSH expression was reduced by siRNA treatment, Nef expression was weaker relative to control and Nef lost its TGN, perinuclear localization. Instead it accumulated in punctated intracellular loci segregated from the TGN.

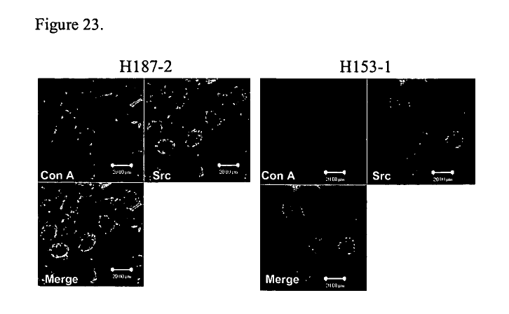

Src is expressed at the plasma membrane and in intracellular vesicles, which are found close to the plasma membrane (FIG. 23, H187 cells). However, when hPOSH levels were reduced, Src was dispersed in the cytoplasm and loses its plasma membrane proximal localization detected in control (H187) cells (FIG. 23, compare H153-1 and H187-2 panels).

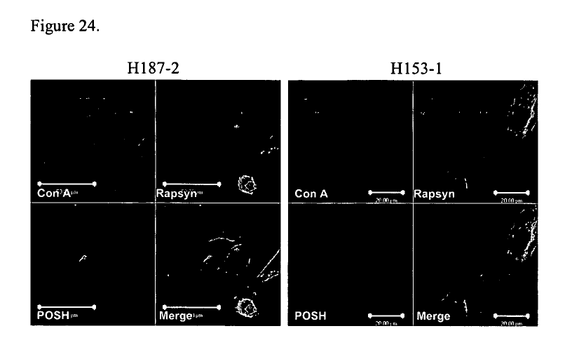

Rapsyn, a peripheral membrane protein expressed in skeletal muscle, plays a critical role in organizing the structure of the nicotinic postsynaptic membrane (Sanes and Lichtman, Annu. Rev. Neurosci. 22: 389-442 (1999)). Newly synthesized Rapsyn associates with the TGN and than transported to the plasma membrane (Marchand et al., J. Neurosci. 22: 8891-01 (2002)). In hPOSH-depleted cells (H153-1) Rapsyn was dispersed in the cytoplasm, while in control cells it had a punctuated pattern and plasma membrane localization, indicating that hPOSH influences its intracellular transport (FIG. 24).

Materials and Methods Used:

Antibodies:

Src antibody was purchased from Oncogene research products( Darmstadt, Germany). Nef antibodies were pusrchased from ABI (Columbia, Mass.) and Fitzgerald Industries Interantional (Concord, Mass.). Alexa Fluor conjugated antibodies were pusrchased from Molecular Probes Inc. (Eugene, Oreg.).

hPOSH antibody: Glutathione S-transferase (GST) fusion plasmids were constructed by PCR amplification of hPOSH codons 285-430. The amplified PCR products was cloned into pGEX-6P-2 (Amersham Pharmacia Biotech, Buckinghamshire, UK). The truncated hPOSH protein was generated in E. coli BL21. Bacterial cultures were grown in LB media with carbenicillin (100 μg/ml) and recombinant protein production was induced with 1 mM IPTG for 4 hours at 30° C. Cells were lysed by sonication and the recombinant protein was then isolated from the cleared bacterial lysate by affinity chromatography on a glutathione-sepharose resin (Amersham Pharmacia Biotech, Buckinghamshire, UK). The hPOSH portion of the fusion protein was then released by incubation with PreScission protease (Amersham Pharmacia Biotech, Buckinghamshire, UK) according to the manufacturer's instructions and the GST portion was then removed by a second glutathione-sepharose affinity chromatography. The purified partial hPOSH polypeptide was used to immunize New Zealand white rabbits to generate antibody 15B (Washington Biotechnology, Baltimore, Md.).

Construction of siRNA Retroviral Vectors:

hPOSH scrambled oligonucleotide (5′-CACACACTGCCG TCAACT GTTCAAGAGAC AGTTGACGGCAGTGTGTGTTTTTT-3′; and 5′-AATTAAAAAACACA CACTGCCGTCAACTGTC TCTTGAACAGTTGA CGGCAGTGTGTGGGCC-3′) were annealed and cloned into the ApaI-EcoRI digested pSilencer 1.0-US (Ambion) to generate pSIL-scrambled. Subsequently, the U6-promoter and RNAi sequences were digested with BamHI, the ends filled in and the insert cloned into the Oli1 site in the retroviral vector, pMSVhyg (Clontech), generating pMSCVhyg-U6-scrambled. hPOSH oligonucleotide encoding RNAi against hPOSH (5′-AACAGAGGCCTTGGAAA CCTGGAAGC TTGCAGGTTT CCAAGGCCTCTGTT-3′; and 5′-GATCAACAGAG GCCTTGGAAACCTGC AAGCTTCCAGGTTTCCAA GGCCTCTGTT-3′) were annealed and cloned into the BamHI-EcoRI site of pLIT-U6, generating pLIT-U6 hPOSH-230. pLIT-U6 is an shRNA vector containing the human U6 promoter (amplified by PCR from human genomic DNA with the primers, 5′-GGCCCACTAGTCA AGGTCG GGCA GGAAGA-3′ and 5′-GCCGAATT CAAAAAGGATC CGGCGATATCCGG TGTTTCGTCCTTTCCA -3′) cloned into pLITMUS38 (New England Biolabs) digested with SpeI-EcoRI. Subsequently, the U6 promoter-hPOSH shRNA (PLIT-U6 hPOSH-230 digested with SnaBI and PvuI) was cloned into the Oli1 site of pMSVhyg (Clontech), generating pMSCVhyg U6-hPOSH-230.

Generation of Stable Clones:

HEK 293T cells were transfected with retroviral RNAi plasmids (pMSCVhyg-U6-Prt3-230 and pMSCVhyg-U6-scrambled and with plasmids encoding VSV-G and moloney gag-pol. Two days post transfection, medium containing retroviruses was collected and filtered and polybrene was added to a final concentration of 8 μg/ml. This was used to infect HeLa SS6 cells grown in 60 mm dishes. Forty-eight hours post-infection cells were selected for RNAi expression by the addition of hygromycin to a final concentration of 300 μg/ml. Clones expressing RNAi against hPOSH were named H153, clones expressing scrambled RNAi were named H187.

Transfection and Immunofluorescent Analysis:

Gag-EGFP experiments are described in FIG. 21.

H153 or H187 cells were transfected with Src or Rapsyn-GFP (Image clone image: 3530551 or pNLenv-1). Eighteen hours post transfection cells were washed with PBS and incubated on ice with Alexa Fluor 647 conjugated Con A to label plasma membrane glycoproteins. Subsequently cells were fixed in 3% paraformaldehyde, blocked with PBS containing 4% bovine serum albumin and 1% gelatin. Staining with rabbit anti-Src, rabbit anti-hPOSH (15B) or mouse anti-nef was followed with secondary antibodies as indicated.

Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown.

### Example 8

### POSH Protein-Protein Interactions by Yeast Two Hybrid Assay

POSH-associated proteins were identified by using a yeast two-hybrid assay.

Procedure:

Bait plasmid (GAL4-BD) was transformed into yeast strain AH109 (Clontech) and transformants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:

Plasmid vector: pGBK-T7 (Clontech)
Plasmid name: pPL269-pGBK-T7 GAL4 POSHdR

Protein sequence: Corresponds to aa 53-888 of POSH (RING domain deleted)

RTLVGSGVEELPSNILLVRLLDGIKQRPWKPGPGGGSGTNCTNALRSQSSTVANCSSKDL
QSSQGGQQPRVQSWSPPVRGIPQLPCAKALYNYEGKEPGDLKFSKGDIIILRRQVDENWY
HGEVNGIHGFFPTNFVQIIKPLPQPPPQCKALYDFEVKDKEADKDCLPFAKDDVLTVIRR
VDENWAEGMLADKIGIFPISYVEFNSAAKQLIEWDKPPVPGVDAGECSSAAAQSSTAPKH
SDTKKNTKKRHSFTSLTMANKSSQASQNRHSMEISPPVLISSSNPTAAARISELSGLSCS
APSQVHISTTGLIVTPPPSSPVTTGPSFTFPSDVPYQAALGTLNPPLPPPPLLAATVLAS
TPPGATAAAAAAGMGPRPMAGSTDQIAHLRPQTRPSVYVAIYPYTPRKEDELELRKGEMF
LVFERCQDGWFKGTSMHTSKIGVFPGNYVAPVTRAVTNASQAKVPMSTAGQTSRGVTMVS
PSTAGGPAQKLQGNGVAGSPSVVPAAVVSAAHIQTSPQAKVLLHMTGQMTVNQARNAVRT
VAAHNQERPTAAVTPIQVQNAAGLSPASVGLSHHSLASPQPAPLMPGSATHTAAISISRA
SAPLACAAAAPLTSPSITSASLEAEPSGRIVTVLPGLPTSPDSASSACGNSSATKPDKDS
KKEKKGLLKLLSGASTKRKPRVSPPASPTLEVELGSAELPLQGAVGPELPPGGGHGRAGS
CPVDGDGPVTTAVAGAALAQDAFHRKASSLDSAVPIAPPPRQACSSLGPVLNESRPVVCE
RHRVVVSYPPQSEAELELKEGDIVFVHKKREDGWFKGTLQRNGKTGLFPGSFVENI

Library screened: Hela pretransformed library (Clontech).

POSH-APs identified by yeast two-hybrid assay include PACS-1, HLA-A, and HLA-B. See International Application No. PCT/US2004/006308, published as WO 2004/078130.

### Example 9

### HPOSH is Phosphorylated by Protein Kinase A (PKA) Materials and Methods

PKA-Dependent Phosphorylation of hPOSH.

Bacterially expressed recombinant maltose-binding-protein (MBP)-hPOSH (3 μg) or GST-c-Cbl were incubated at 30° C. for 30 minutes with (*) or without 10 ng PKA catalytic subunit (PKAc) in a buffer containing 40 mM Tris-HCl pH 7.4, 10 mM MgCl₂, 4 mM ATP, 0.1 mg/ml BSA, 1 μM cAMP, 23 mM K₃PO₄, 7 nM DTT, and PKA peptide protection solution (Promega, Cat.No. V5340). The reaction was stopped by the addition of SDS-sample buffer, and boiling for 3 minutes. Samples were separated by SDS-PAGE on a 10% gel, and transferred to nitrocellulose and immunoblotted as detailed in the figure.

Binding of Rac1 to hPOSH

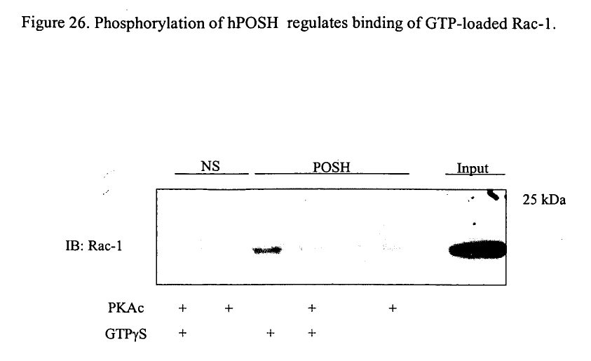

Bacterially expressed hPOSH (1 μg) or GST (1 μg) were phosphorylated as above. The reaction was terminated by the addition 0.5 ml of ice-cold 200 mM Tris-HCl pH 7.4, 5 mM EDTA. hPOSH and GST were then immobilized on NiNTA or reduced glutathione beads, respectively, by gentle mixing for 30 minutes. The immobilized proteins were washed three times with wash buffer (50 mM Tris-HCl pH 7.4, 100 mM NaCl, 5 mM MgCl2, 0.1 mM DTT). Recombinant Rac-1 (0.2 μ) (Sigma catalog #R3012) was incubated with or without 0.3 mM GTPγS (Sigma Cat. No. G8638) on ice for 15 minutes. The GTP/mock-loaded Rac-1 was then added to wash buffer (25 μl, final) and incubated for 30 minutes at 30° C. The beads were then washed three times with wash buffer containing 0.1% Tween 20. Sample buffer was added to the bead pellet and boiled for 3 minutes. Immobilized and associating proteins were then separated by SDS-PAGE on a 12% gel and immunobloted with anti-Rac-1 (Santa Cruz Biotechnology, Cat. No. sc-217). Input is 0.25 μg of Rac-1. See FIG. 26.

### Example 10

### Nef Protein Levels are Undetectable in Cell Lines Expressing RNAi Directed Against hPOSH

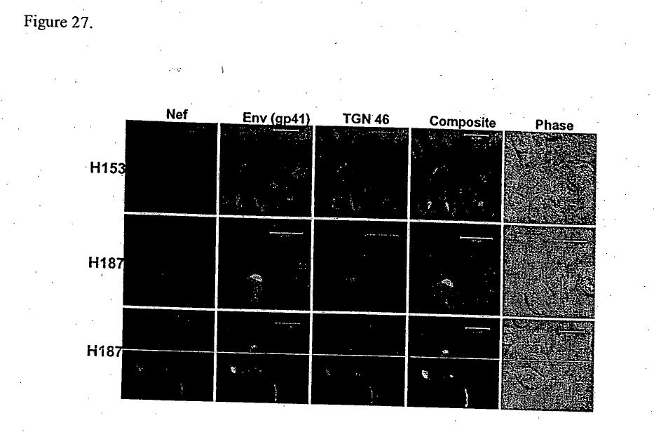

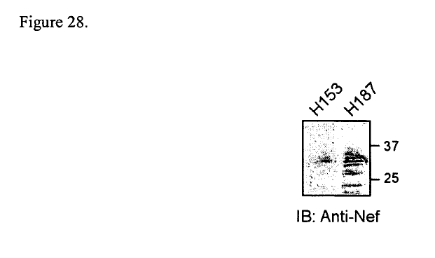

Stable Hela SS6 cell lines expressing RNAi directed against hPOSH or control RNAi were generated (H153 and H187, respectively). H153 and H187 cells were transfected with a plasmid encoding HIV-1 and Nef protein levels were assessed by immunoblot and immunofluorescence analysis. In H153 cells Nef protein was undetectable by both methods (FIGS. 27 and 28), while other viral proteins are expressed, for example gag, env and vpu (FIG. 27).

### Example 11

### POSH Protein-Protein Interactions by Yeast Two Hybrid Assay

POSH-associated proteins were identified by using a yeast two-hybrid assay.

Procedure:

Bait plasmid (GAL4-BD) was transformed into yeast strain AH109 (Clontech) and transformants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:

Plasmid vector: pGBK-T7 (Clontech)
Plasmid name: pPL269-pGBK-T7 GAL4 POSHdR

Protein sequence: Corresponds to aa 53-888 of POSH (RING domain deleted)

RTLVGSGVEELPSNILLVRLLDGIKQRPWKPGPGGGSGTNCTNALRSQSSTVANCSSKDL
QSSQGGQQPRVQSWSPPVRGIPQLPCAKALYNYEGKEPGDLKFSKGDIIILRRQVDENWY
HGEVNGIHGFFPTNFVQIIKPLPQPPPQCKALYDFEVKDKEADKDCLPFAKDDVLTVIRR
VDENWAEGMLADKIGIFPISYVEFNSAAKQLIEWDKPPVPGVDAGECSSAAAQSSTAPKH
SDTKKNTKKRHSFTSLTMANKSSQASQNRHSMEISPPVLISSSNPTAAARISELSGLSCS
APSQVHISTTGLIVTPPPSSPVTTGPSFTFPSDVPYQAALGTLNPPLPPPPLLAATVLAS
TPPGATAAAAAAGMGPRPMAGSTDQIAHLRPQTRPSVYVAIYPYTPRKEDELELRKGEMF
LVFERCQDGWFKGTSMHTSKIGVFPGNYVAPVTRAVTNASQAKVPMSTAGQTSRGVTMVS
PSTAGGPAQKLQGNGVAGSPSVVPAAVVSAAHIQTSPQAKVLLHMTGQMTVNQARNAVRT
VAAHNQERPTAAVTPIQVQNAAGLSPASVGLSHHSLASPQPAPLMPGSATHTAAISISRA
SAPLACAAAAPLTSPSITSASLEAEPSGRIVTVLPGLPTSPDSASSACGNSSATKPDKDS
KKEKKGLLKLLSGASTKRKPRVSPPASPTLEVELGSAELPLQGAVGPELPPGGGHGRAGS
CPVDGDGPVTTAVAGAALAQDAFHRKASSLDSAVPIAPPPRQACSSLGPVLNESRPVVCE
RHRVVVSYPPQSEAELELKEGDIVFVHKKREDGWFKGTLQRNGKTGLFPGSFVENI

Library screened: Hela pretransformed library (Clontech).

The POSH-AP, HERPUD1 (Hs.146393), was identified by yeast two-hybrid assay. Examples of nucleic acid and amino acid sequences of HERPUD1 are provided below.

Human HERPUD1 mRNA sequence - var1 (public gi: 16507801)
(SEQ ID NO: 37)
AGAGACGTGAACGGTCGTTGCAGAGATTGCCGGCGGCTGAGACGCCGCCTGCCTGGCACCTAGGAGCGCA
GCGGAGCCCCGACACCGCCGCCGCCGCCATGGAGTCCGAGACCGAACCCGAGCCCGTCACGCTCCTGGTG
AAGAGCCCCAACCAGCGCCACCGCGACTTGGAGCTGAGTGGCGACCGCGGCTGGAGTGTGGGCCACCTCA
AGGCCCACCTGAGCCGCGTCTACCCCGAGCGTCCGCGTCCAGAGGACCAGAGGTTAATTTATTCTGGGAA
GCTGTTGTTGGATCACCAATGTCTCAGGGACTTGCTTCCAAAGGAAAAACGGCATGTTTTGCATCTGGTG
TGCAATGTGAAGAGTCCTTCAAAAATGCCAGAAATCAACGCCAAGGTGGCTGAATCCACAGAGGAGCCTG
CTGGTTCTAATCGGGGACAGTATCCTGAGGATTCCTCAAGTGATGGTTTAAGGCAAAGGGAAGTTCTTCG
GAACCTTTCTTCCCCTGGATGGGAAAACATCTCAAGGCATCACGTTGGGTGGTTTCCATTTAGACCGAGG
CCGGTTCAGAACTTCCCAAATGATGGTCCTCCTCCTGACGTTGTAAATCAGGACCCCAACAATAACTTAC
AGGAAGGCACTGATCCTGAAACTGAAGACCCCAACCACCTCCCTCCAGACAGGGATGTACTAGATGGCGA
GCAGACCAGCCCCTCCTTTATGAGCACAGCATGGCTTGTCTTCAAGACTTTCTTTGCCTCTCTTCTTCCA
GAAGGCCCCCCAGCCATCGCAAACTGATGGTGTTTGTGCTGTAGCTGTTGGAGGCTTTGACAGGAATGGA
CTGGATCACCTGACTCCAGCTAGATTGCCTCTCCTGGACATGGCAATGATGAGTTTTTAAAAAACAGTGT
GGATGATGATATGCTTTTGTGAGCAAGCAAAAGCAGAAACGTGAAGCCGTGATACAAATTGGTGAACAAA
AAATGCCCAAGGCTTCTCATGTCTTTATTCTGAAGAGCTTTAATATATACTCTATGTAGTTTAATAAGCA
CTGTACGTAGAAGGCCTTAGGTGTTGCATGTCTATGCTTGAGGAACTTTTCCAAATGTGTGTGTCTGCAT
GTGTGTTTGTACATAGAAGTCATAGATGCAGAAGTGGTTCTGCTGGTACGATTTGATTCCTGTTGGAATG
TTTAAATTACACTAAGTGTACTACTTTATATAATCAATGAAATTGCTAGACATGTTTTAGCAGGACTTTT
CTAGGAAAGACTTATGTATAATTGCTTTTTAAAATGCAGTGCTTTACTTTAAACTAAGGGGAACTTTGCG
GAGGTGAAAACCTTTGCTGGGTTTTCTGTTCAATAAAGTTTTACTATGAATGACCCTGAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAA
Human HERPUD1 mRNA sequence - var2 (public gi: 10441910)
(SEQ ID NO: 38)
GCTGTGTGGCCCAGGCTTTTCTCAAACTCCTGAGGGCAAGCGATCCTCCCACCTCAGCCTCCTGAGTAGC
TGGGACTACAGGCATGTGCCACTAGACCTGGCTCTAAAGACATATATGACACACGAAACCATTTATTTTT
CATTTCACAATGTTTATTCACATATATGGTATTAGTATTCTAATGTAGTGATGCACTCTAAATTTGCATT
ATATTTCCTAGAACATCTGAACAGAGCATAGGAAATTCCCTATTTTGCCATTATCAGTTCTAACAAAAAT
CTTAAAAGCACTTTATCATTTCATTTCCCTGCACTGTAATTTTTTTAAATGATCAAAAACAGTATCATAC
CAAGGCTTACTTATATTGGAATACTATTTTAGAAAGTTGTGGGCTGGGTTGTATTTATAAATCTTGTTGG
TCAGATGTCTGCAATGAGTAAATTTAGCACCATTATCAGGAAGCTTTCTCACCAATGACAACTTCATTGG
AAGATTTTAATGAAAGTGTAGCATACTCTAGGGAAAAAATATGAATATTTTAGCATCTATGTATTGAAAA
TTATGTTGAATAAATGTCAGACTATTTTTTACATAACGTTGCTTCTGTTTAATTTTGTCACGTTCAGAGG
TGGGGGGTAGGAGATGTAAGCCCTTGACAGCAAAATAATTCCTTTTGCTTGATTTCAGACAGTTGCATCA
GCTCCTTTGTTCTGTGTTCATGTTACACTTATTTAGGTGGCTGAATCCACAGAGGAGCCTGCTGGTTCTA
ATCGGGGACAGTATCCTGAGGATTCCTCAAGTGATGGTTTAAGGCAAAGGGAAGTTCTTCGGAACCTTTC
TTCCCCTGGATGGGAAAACATCTCAAGGCCTGAAGCTGCCCAGCAGGCATTCCAAGGCCTGGGTCCTGGT
TTCTCCGGTTACACACCCTATGGGTGGCTTCAGCTTTCCTGGTTCCAGCAGATATATGCACGACAGTACT
ACATGCAATATTTAGCAGCCACTGCTGCATCAGGGGCTTTTGTTCCACCACCAAGTGCACAAGAGATACC
TGTGGTCTCTGCACCTGCTCCAGCCCCTATTCACAACCAGTTTCCAGCTGAAAACCAGCCTGCCAATCAG
AATGCTGCTCCTCAAGTGGTTGTTAATCCTGGAGCCAATCAAAATTTGCGGATGAATGCACAAGGTGGCC
CTATTGTGGAAGAAGATGATGAAATAAATCGAGATTGGTTGGATTGGACCTATTCAGCAGCTACATTTTC
TGTTTTTCTCAGTATCCTCTACTTCTACTCCTCCCTGAGCAGATTCCTCATGGTCATGGGGGCCACCGTT
GTTATGTACCTGCATCACGTTGGGTGGTTTCCATTTAGACCGAGGCCGGTTCAGAACTTCCCAAATGATG
GTCCTCCTCCTGACGTTGTAAATCAGGACCCCAACAATAACTTACAGGAAGGCACTGATCCTGAAACTGA
AGACCCCAACCACCTCCCTCCAGACAGGGATGTACTAGATGGCGAGCAGACCAGCCCCTCCTTTATGAGC
ACAGCATGGCTTGTCTTCAAGACTTTCTTTGCCTCTCTTCTTCCAGAAGGCCCCCCAGCCATCGCAAACT
GATGGTGTTTGTGCTGTAGCTGTTGGAGGCTTTGACAGGAATGGACTGGATCACCTGACTCCAGCTAGAT
TGCCTCTCCTGGACATGGCAATGATGAGTTTTTAAAAAACAGTGTGGATGATGATATGCTTTTGTGAGCA
AGCAAAAGCAGAAACGTGAAGCCGTGATACAAATTGGTGAACAAAAAATGCCCAAGGCTTCTCATGTCTT
TATTCTGAAGAGCTTTAATATATACTCTATGTAGTTTAATAAGCACTGTACGTAGAAGGCCTTAGGTGTT
GCATGTCTATGCTTGAGGAACTTTTCCAAATGTGTGTGTCTGCATGTGTGTTTGTACATAGAAGTCATAG
ATGCAGAAGTGGTTCTGCTGGTACGATTTGATTCCTGTTGGAATGTTTAAATTACACTAAGTGTACTACT
TTATATAATCAATGAAATTGCTAGACATGTTTTAGCAGGACTTTTCTAGGAAAGACTTATGTATAATTGC
TTTTTAAAATGCAGTGCTTTACTTTAAACTAAGGGGAACTTTGCGGAGGTGAAAACCTTTGCTGGGTTTT
CTGTTCAATAAAGTTTTACTATGAATGACAAAAAAAAAAAAAAAAAA
Human HERPUD1 mRNA sequence - var3 (public gi: 3005722)
(SEQ ID NO: 39)
GGCCACCTCAAGGCCCACCTGAGCCGCGTCTACCCCGAGCGTCCGCGTCCAGAGGACCAGAGGTTAATTT
ATTCTGGGAAGCTGTTGTTGGATCACCAATGTCTCAGGGACTTGCTTCCAAAGGAAAAACGGCATGTTTT
GCATCTGGTGTGCAATGTGAAGAGTCCTTCAAAAATGCCAGAAATCAACGCCAAGGTGGCTGAATCCACA
GAGGAGCCTGCTGGTTCTAATCGGGGACAGTATCCTGAGGATTCCTCAAGTGATGGTTTAAGGCAAAGGG
AAGTTCTTCGGAACCTTTCTTCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTCAGCTTTCCTGGTTCCAGCAG
ATATATGCACGACAGTACTACATGCAATATTTAGCAGCCACTGCTGCATCAGGGGCTTTTGTTCCACCAC
CAAGTGCACAAGAGATACCTGTGGTCTCTGCACCTGCTCCAGCCCCTATTCACAACCAGTTTCCAGCTGA
AAACCAGCCTGCCAATCAGAATGCTGCTCCTCAAGTGGTTGTTAATCCTGGAGCCAATCAAAATTTGCGG
ATGAATGCACAAGGTGGCCCTATTGTGGAAGAAGATGATGAAATAAATCGAGATTGGTTGGATTGGACCT
ATTCAGCAGCTACATTTTCTGTTTTTCTCAGTATCCTCTACTTCTACTCCTCCCTGAGCAGATTCCTCAT
GGTCATGGGGGCCACCGTTGTTATGTACCTGCATCACGTTGGGTGGTTTCCATTTAGACCGAGGCCGGTT
CAGAACTTCCCAAATGATGGTCCTCCTCCTGACGTTGTAAATCAGGACCCCAACAATAACTTACAGGAAG
GCACTGATCCTGAAACTGAAGACCCCAACCACCTCCCTCCAGACAGGGATGTACTAGATGGCGAGCAGAC
CAGCCCCTCCTTTATGAGCACAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCTTTGACAGGAATGGACTGGAT
CACCTGACTCCAGCTAGATTGCCTCTCCTGGACATGGCAATGATGAGTTTTTAAAAAACAGTGTGGATGA
TGATATGCTTTTGTGAGCAAGCAAAAGCAGAAACGTGAAGCCGTGATACAAATTGGTGAACAAAAAATGC
CCAAGGCTTCTCATGTCTTTATTCTGAAGAGCTTTAATATATACTCTATGTAGTTTAATAAGCACTGTAC
GTAGAAGGCCTTAGGTGTTGCATGTCTATGCTTGAGGAACTTTTCCAAATGTGTGTGTCTGCATGTGTGT
TTGTACATAGAAGTCATAGATGCAGAAGTGGTTCTGCTGGTACGATTTGATTCCTGTTGGAATGTTTAAA
TTACACTAAGTGTACTACTTTATATAATCAATGAAATTGCTAGACATGTTTTAGCAGGACTTTTCTAGGA
AAGACTTATGTATAATTGCTTTTTAAAATGCAGTGCTTTACTTTAAACTAAGGGGAACTTTGCGGAGGTG
AAAACCTTTGCTGGGTTTTCTGTTCAATAAAGTTTTACTATGAATGACCCTGAAAAAAAAAAAAAAAAAA
Human HERPUD1 mRNA sequence - var4 (public gi: 21619176)
(SEQ ID NO: 40)
CCACGCGTCCGGGTCGTTGCAGAGATTGCGGGCGGCTGAGACGCCGCCTGCCTGGCACCTAGGAGCGCAG
CGGAGCCCCGACACCGCCGCCGCCGCCATGGAGTCCGAGACCGAACCCGAGCCCGTCACGCTCCTGGTGA
AGAGCCCCAACCAGCGCCACCGCGACTTGGAGCTGAGTGGCGACCGCGGCTGGAGTGTGGGCCACCTCAA
GGCCCACCTGAGCCGCGTCTACCCCGAGCGTCCGCGTCCAGAGGACCAGAGGTTAATTTATTCTGGGAAG
CTGTTGTTGGATCACCAATGTCTCAGGGACTTGCTTCCAAAGCAGGAAAAACGGCATGTTTTGCATCTGG
TGTGCAATGTGAAGAGTCCTTCAAAAATGCCAGAAATCAACGCCAAGGTGGCTGAATCCACAGAGGAGCC
TGCTGGTTCTAATCGGGGACAGTATCCTGAGGATTCCTCAAGTGATGGTTTAAGGCAAAGGGAAGTTCTT
CGGAACCTTTCTTCCCCTGGATGGGAAAACATCTCAAGGCCTGAAGCTGCCCAGCAGGCATTCCAAGGCC
TGGGTCCTGGTTTCTCCGGTTACACACCCTATGGGTGGCTTCAGCTTTCCTGGTTCCAGCAGATATATGC
ACGACAGTACTACATGCAATATTTAGCAGCCACTGCTGCATCAGGGGCTTTTGTTCCACCACCAAGTGCA
CAAGAGATACCTGTGGTCTCTGCACCTGCTCCAGCCCCTATTCACAACCAGTTTCCAGCTGAAAACCAGC
CTGCCAATCAGAATGCTGCTCCTCAAGTGGTTGTTAATCCTGGAGCCAATCAAAATTTGCGGATGAATGC
ACAAGGTGGCCCTATTGTGGAAGAAGATGATGAAATAAATCGAGATTGGTTGGATTGGACCTATTCAGCA
GCTACATTTTCTGTTTTTCTCAGTATCCTCTACTTCTACTCCTCCCTGAGCAGATTCCTCATGGTCATGG
GGGCCACCGTTGTTATGTACCTGCATCACGTTGGGTGGTTTCCATTTAGACCGAGGCCGGTTCAGAACTT
CCCAAATGATGGTCCTCCTCCTGACGTTGTAAATCAGGACCCCAACAATAACTTACAGGAAGGCACTGAT
CCTGAAACTGAAGACCCCAACCACCTCCCTCCAGACAGGGATGTACTACATGGCGAGCAGACCAGCCCCT
CCTTTATGAGCACAGCATGGCTTGTCTTCAAGACTTTCTTTGCCTCTCTTCTTCCAGAAGGCCCCCCAGC
CATCGCAAACTGATGGTGTTTGTGCTGTAGCTGTTGGAGGCTTTGACAGGAATGGACTGGATCACCTGAC
TCCAGCTAGATTGCCTCTCCTGGACATGGCAATGATGAGTTTTTAAAAAACAGTGTGGATGATGATATGC
TTTTGTGAGCAAGCAAAGCAGAAACGTGAAGCCGTGATACAAATTGGTGAACAAAAAATGCCCAAGGCTT
CTCATGTCTTTATTCTGAAGAGCTTTAATATATACTCTATGTAGTTTAATAAGCACTGTACGTAGAAGGC
CTTAGGTGTTGCATGTCTATGCTTGAGGAACTTTTCCAAATGTGTGTGTCTGCATGTGTGTTTGTACATA
GAAGTCATAGATGCAGAAGTGGTTCTGCTGGTACGATTTGATTCCTGTTGGAATGTTTAAATTACACTAA
GTGTACTACTTTATATAATCAATGAAATTGCTAGACATGTTTTAGCAGGACTTTTCTAGGAAAGACTTAT
GTATAATTGCTTTTTAAAATGCAGTCCTTTACTTTAAACTAAGGGGAACTTTGCGGAGGTGAAAACCTTT
GCTGGGTTTTCTGTTCAATAAAGTTTTACTATGAATGACCCTGAAAAAAAAAAAAAAA
Human HERPUD1 mRNA sequence - var5 (public gi: 14249882)
(SEQ ID NO: 41)
AACGGTCGTTGCAGAGATTGCGGGCGGCTGAGACGCCGCCTGCCTGGCACCTAGGAGCGCAGCGGAGCCC
CGACACCGCCGCCGCCGCCATGGAGTCCGAGACCGAACCCGAGCCCGTCACGCTCCTGGTGAAGAGCCCC
AACCAGCGCCACCGCGACTTGGAGCTGAGTGGCGACCGCGGCTGGAGTGTGGGCCACCTCAAGGCCCACC
TGAGCCGCGTCTACCCCGAGCGTCCGCGTCCAGAGGACCAGAGGTTAATTTATTCTGGGAAGCTGTTGTT
GGATCACCAATGTCTCAGGGACTTGCTTCCAAAGCAGGAAAAACGGCATGTTTTGCATCTGGTGTGCAAT
GTGAAGAGTCCTTCAAAAATGCCAGAAATCAACGCCAAGGTGGCTGAATCCACAGAGGAGCCTGCTGGTT
CTAATCGGGGACAGTATCCTGAGGATTCCTCAAGTGATGGTTTAAGGCAAAGGGAAGTTCTTCGGAACCT
TTCTTCCCCTGGATGGGAAAACATCTCAAGGCCTGAAGCTGCCCAGCAGGCATTCCAAGGCCTGGGTCCT
GGTTTCTCCGGTTACACACCCTATGGGTGGCTTCAGCTTTCCTGGTTCCAGCAGATATATGCACGACAGT
ACTACATGCAATATTTAGCAGCCACTGCTGCATCAGGGGCTTTTGTTCCACCACCAAGTGCACAAGAGAT
ACCTGTGGTCTCTGCACCTGCTCCAGCCCCTATTCACAACCAGTTTCCAGCTGAAAACCAGCCTGCCAAT
CAGAATGCTGCTCCTCAAGTGGTTGTTAATCCTGGAGCCAATCAAAATTTGCGGATGAATGCACAAGGTG
GCCCTATTGTGGAAGAAGATGATGAAATAAATCGAGATTGGTTGGATTGGACCTATTCAGCAGCTACATT
TTCTGTTTTTCTCAGTATCCTCTACTTCTACTCCTCCCTGAGCAGATTCCTCATGGTCATGGGGGCCACC
GTTGTTATGTACCTGCATCACGTTGCGTGGTTTCCATTTAGACCGAGGCCGGTTCAGAACTTCCCAAATG
ATGGTCCTCCTCCTGACGTTGTAAATCAGGACCCCAACAATAACTTACAGGAAGGCACTGATCCTGAAAC
TGAAGACCCCAACCACCTCCCTCCAGACAGGGATGTACTAGATGGCGAGCAGACCAGCCCCTCCTTTATG
AGCACAGCATGGCTTGTCTTCAAGACTTTCTTTGCCTCTCTTCTTCCAGAAGGCCCCCCAGCCATCGCAA
ACTGATGGTGTTTGTGCTGTAGCTGTTGGAGGCTTTGACAGGAATGGACTGGATCACCTGACTCCAGCTA
GATTGCCTCTCCTGGACATGGCAATGATGAGTTTTTAAAAAACAGTGTGGATGATGATATGCTTTTGTGA
GCAAGCAAAAGCAGAAACGTGAAGCCGTGATACAAATTGGTGAACAAAAAATGCCCAAGGCTTCTCATGT
CTTTATTCTGAAGAGCTTTAATATATACTCTATGTAGTTTAATAAGCACTGTACGTAGAAGGCCTTAGGT
GTTGCATGTCTATGCTTGAGGAACTTTTCCAAATGTGTGTGTCTGCATGTGTGTTTGTACATAGAAGTCA
TAGATGCAGAAGTGGTTCTGCTGGTACGATTTGATTCCTGTTGGAATGTTTAAATTACACTAAGTGTACT
ACTTTATATAATCAATGAAATTGCTAGACATGTTTTAGCAGGACTTTTCTAGGAAAGACTTATGTATAAT
TGCTTTTTAAAATGCAGTGCTTTACTTTAAACTAAGGGGAACTTTGCGGAGGTGAAAACCTTTGCTGGGT
TTTCTGTTCAATAAAGTTTTACTATGAAAAAAAAAAAAAAAAAA
Human HERPUD1 mRNA sequence - var6 (public gi: 12652674)
(SEQ ID NO: 42)
GAACTGTCGTTGCAGAGATTGCGGGCGGCTGAGACGCCGCCTGCCTGGCACCTAGGAGCGCAGCGGAGCC
CCGACACCGCCGCCGCCGCCATGGAGTCCGAGACCGAACCCGAGCCCGTCACGCTCCTGGTGAAGAGCCC
CAACCAGCGCCACCGCGACTTGGAGCTGAGTGGCGACCGCGGCTGGAGTGTGGGCCACCTCAAGGCCCAC
CTGAGCCGCGTCTACCCCGAGCGTCCGCGTCCAGAGGACCAGAGGTTAATTTATTCTGGGAAGCTGTTGT
TGGATCACCAATGTCTCAGGGACTTGCTTCCAAAGCAGGAAAAACGGCATGTTTTGCATCTGGTGTCCAA
TGTGAAGAGTCCTTCAAAAATGCCAGAAATCAACGCCAAGGTGGCTGAATCCACAGAGGAGCCTGCTGGT
TCTAATCGGGGACAGTATCCTGAGGATTCCTCAAGTGATGGTTTAAGGCAAAGGGAAGTTCTTCGGAACC
TTTCTTCCCCTGGATGGGAAAACATCTCAAGGCCTGAAGCTGCCCAGCAGGCATTCCAAGGCCTGGGTCC
TGGTTTCTCCGGTTACACACCCTATGGGTGGCTTCAGCTTTCCTGGTTCCAGCAGATATATGCACGACAG
TACTACATGCAATATTTAGCAGCCACTGCTGCATCAGGGGCTTTTGTTCCACCACCAAGTGCACAAGAGA
TACCTGTGGTCTCTGCACCTGCTCCAGCCCCTATTCACAACCAGTTTCCAGCTGAAAACCAGCCTGCCAA
TCAGAATGCTGCTCCTCAAGTGGTTGTTAATCCTGGAGCCAATCAAAATTTGCGGATGAATGCACAAGGT
GGCCCTATTGTGGAAGAAGATGATGAAATAAATCGAGATTGGTTGGATTGGACCTATTCAGCAGCTACAT
TTTCTGTTTTTCTCAGTATCCTCTACTTCTACTCCTCCCTGAGCAGATTCCTCATGGTCATGGGGGCCAC
CGTTGTTATGTACCTGCATCACGTTGGGTGGTTTCCATTTAGACCGAGGCCGGTTCAGAACTTCCCAAAT
GATGGTCCTCCTCCTGACGTTGTAAATCAGGACCCCAACAATAACTTACAGGAAGGCACTGATCCTGAAA
CTGAAGACCCCAACCACCTCCCTCCAGACAGGGATGTACTAGATGGCGAGCAGACCAGCCCCTCCTTTAT
GAGCACAGCATGGCTTGTCTTCAAGACTTTCTTTGCCTCTCTTCTTCCAGAAGGCCCCCCAGCCATCGCA
AACTGATGGTGTTTGTGCTGTAGCTGTTGGAGGCTTTGACAGGAATGGACTGGATCACCTGACTCCAGCT
AGATTGCCTCTCCTGGACATGGCAATGATGAGTTTTTAAAAAACAGTGTGGATGATGATATGCTTTTGTG
AGCAAGCAAAAGCAGAAACGTGAAGCCGTGATACAAATTGGTGAACAAAAAATGCCCAAGGCTTCTCATG
TCTTTATTCTGAAGAGCTTTAATATATACTCTATGTAGTTTAATAAGCACTGTACGTAGAAGGCCTTAGG
TGTTGCATGTCTATGCTTGAGGAACTTTTCCAAATGTGTGTGTCTGCATGTGTGTTTGTACATAGAAGTC
ATAGATGCAGAAGTGGTTCTGCTGGTACGATTTGATTCCTGTTGGAATGTTTAAATTACACTAAGTGTAC
TACTTTATATAATCAATGAAATTGCTAGACATGTTTTAGCAGGACTTTTCTAGGAAAGACTTATGTATAA
TTGCTTTTTAAAATGCAGTGCTTTACTTTAAACTAAGGGGAACTTTGCGGAGGTGAAAACCTTTGCTGGG
TTTTCTGTTCAATAAAGTTTTACTATGAATGAAAAAAAAAAAAAAAAAAAA
Human HERPUD1 mRNA sequence - var7 (public gi: 9711684)
(SEQ ID NO: 43)
AGAGACGTGAACTGTCGTTGCAGAGATTGCGGGCGGCTGAGACGCCGCCTGCCTGGCACCTAGGAGCGCA
GCGGAGCCCCGACACCGCCGCCGCCGCCATGGAGTCCGAGACCGAACCCGAGCCCGTCACGCTCCTGGTG
AAGAGCCCCAACCAGCGCCACCGCGACTTGGAGCTGAGTGGCGACCGCGGCTGGAGTGTGGGCCACCTCA
AGGCCCACCTGAGCCGCGTCTACCCCGAGCGTCCGCGTCCAGAGGACCAGAGGTTAATTTATTCTGGGAA
GCTGTTGTTGGATCACCAATGTCTCACGGACTTGCTTCCAAAGCAGGAAAAACGGCATGTTTTGCATCTG
GTGTGCAATGTGAAGAGTCCTTCAAAAATGCCAGAAATCAACGCCAAGGTGGCTGAATCCACAGAGGAGC
CTGCTGGTTCTAATCGGGGACAGTATCCTGAGGATTCCTCAAGTGATGGTTTAAGGCAAAGGGAAGTTCT
TCGGAACCTTTCTTCCCCTGGATGGGAAAACATCTCAAGGCCTGAAGCTGCCCAGCAGGCATTCCAAGGC
CTGGGTCCTGGTTTCTCCGGTTACACACCCTATGGGTGGCTTCAGCTTTCCTGGTTCCAGCAGATATATG
CACGACAGTACTACATGCAATATTTAGCAGCCACTGCTGCATCAGGGGCTTTTGTTCCACCACCAAGTGC
ACAAGAGATACCTGTGGTCTCTGCACCTGCTCCAGCCCCTATTCACAACCAGTTTCCAGCTGAAAACCAG
CCTGCCAATCAGAATCCTGCTCCTCAAGTGGTTGTTAATCCTGGAGCCAATCAAAATTTGCGGATGAATG
CACAAGGTGGCCCTATTGTGGAAGAAGATGATGAAATAAATCGAGATTGGTTGGATTGGACCTATTCAGC
AGCTACATTTTCTGTTTTTCTCAGTATCCTCTACTTCTACTCCTCCCTGAGCAGATTCCTCATGGTCATG
GGGGCCACCGTTGTTATGTACCTGCATCACGTTGGGTGGTTTCCATTTAGACCGAGGCCGGTTCAGAACT
TCCCAAATGATGGTCCTCCTCCTGACGTTGTAAATCAGGACCCCAACAATAACTTACAGGAAGGCACTGA
TCCTGAAACTGAAGACCCCAACCACCTCCCTCCAGACAGGGATGTACTAGATGGCGAGCAGACCAGCCCC
TCCTTTATGAGCACAGCATGGCTTGTCTTCAAGACTTTCTTTGCCTCTCTTCTTCCAGAAGGCCCCCCAG
CCATCGCAAACTGATGGTGTTTGTGCTGTAGCTGTTGGAGGCTTTGACAGGAATGGACTGGATCACCTGA
CTCCAGCTAGATTGCCTCTCCTGGACATGGCAATGATGAGTTTTTAAAAAACAGTGTGGATGATGATATG
CTTTTGTGAGCAAGCAAAAGCAGAAACGTGAAGCCGTGATACAAATTGGTGAACAAAAAATGCCCAAGGC
TTCTCATGTCTTTATTCTGAAGAGCTTTAATATATACTCTATGTAGTTTAATAAGCACTGTACGTAGAAG
GCCTTAGGTGTTGCATGTCTATGCTTGAGGAACTTTTCCAAATGTGTGTGTCTGCATGTGTGTTTGTACA
TAGAAGTCATAGATGCAGAAGTGGTTCTGCTGGTACGATTTGATTCCTGTTGGAATGTTTAAATTACACT
AAGTGTACTACTTTATATAATCAATGAAATTGCTAGACATGTTTTAGCAGGACTTTTCTAGGAAAGACTT
ATGTATAATTGCTTTTTAAAATGCAGTGCTTTACTTTAAACTAAGGGGAACTTTGCGGAGGTGAAAACCT
TTGCTGGGTTTTCTGTTCAATAAAGTTTTACTATGAATGACCCTG
Human HERPUD1 mRNA sequence - var8 (public gi: 3005718)
(SEQ ID NO: 44)
GACGTGAACGGTCGTTGCAGAGATTGCGGGCGGCTGAGACGCCGCCTGCCTGGCACCTAGGAGCGCAGCG
GAGCCCCGACACCGCCGCCGCCGCCATGGAGTCCGAGACCGAACCCGAGCCCGTCACGCTCCTGGTGAAG
AGCCCCAACCAGCGCCACCGCGACTTGGAGCTGAGTGGCGACCGCGGCTGGAGTGTGGGCCACCTCAAGG
CCCACCTGAGCCGCGTCTACCCCGAGCGTCCGCGTCCAGAGGACCAGAGGTTAATTTATTCTGGGAAGCT
GTTGTTGGATCACCAATGTCTCAGGGACTTGCTTCCAAAGCAGGAAAAACGGCATGTTTTGCATCTGGTG
TGCAATGTGAAGAGTCCTTCAAAAATGCCAGAAATCAACGCCAAGGTGGCTGAATCCACAGAGGAGCCTG
CTGGTTCTAATCGGGGACAGTATCCTGAGGATTCCTCAAGTGATGGTTTAAGGCAAAGGGAAGTTCTTCG
GAACCTTTCTTCCCCTGGATGGGAAAACATCTCAAGGCCTGAAGCTGCCCAGCAGGCATTCCAAGGCCTG
GGTCCTGGTTTCTCCGGTTACACACCCTATGGGTGGCTTCAGCTTTCCTGGTTCCAGCAGATATATGCAC
GACAGTACTACATGCAATATTTAGCAGCCACTGCTGCATCAGGGGCTTTTGTTCCACCACCAAGTGCACA
AGAGATACCTGTGGTCTCTGCACCTGCTCCAGCCCCTATTCACAACCAGTTTCCAGCTGAAAACCAGCCT
GCCAATCAGAATGCTGCTCCTCAAGTGGTTGTTAATCCTGGAGCCAATCAAAATTTGCGGATGAATGCAC
AAGGTGGCCCTATTGTGGAAGAAGATGATGAAATAAATCGAGATTGGTTGGATTGGACCTATTCAGCAGC
TACATTTTCTGTTTTTCTCAGTATCCTCTACTTCTACTCCTCCCTGAGCAGATTCCTCATGGTCATGGGG
GCCACCGTTGTTATGTACCTGCATCACGTTGGGTGGTTTCCATTTAGACCGAGGCCGGTTCAGAACTTCC
CAAATGATGGTCCTCCTCCTGACGTTGTAAATCAGGACCCCAACAATAACTTACAGGAAGGCACTGATCC
TGAAACTGAAGACCCCAACCACCTCCCTCCAGACAGGGATGTACTAGATGGCGAGCAGACCAGCCCCTCC
TTTATGAGCACAGCATGGCTTGTCTTCAAGACTTTCTTTGCCTCTCTTCTTCCAGAAGGCCCCCCAGCCA
TCGCAAACTGATGGTGTTTGTGCTGTAGCTGTTGGAGGCTTTGACAGGAATGGACTGGATCACCTGACTC
CAGCTAGATTGCCTCTCCTGGACATGGCAATGATGAGTTTTTAAAAAACAGTGTGGATGATGATATGCTT
TTGTGAGCAAGCAAAAGCAGAAACGTGAAGCCGTGATACAAATTGGTGAACAAAAAATGCCCAAGGCTTC
TCATGTCTTTATTCTGAAGAGCTTTAATATATACTCTATGTAGTTTAATAAGCACTGTACGTAGAAGGCC
TTAGGTGTTGCATGTCTATGCTTGAGGAACTTTTCCAAATGTGTGTGTCTGCATGTGTGTTTGTACATAG
AAGTCATAGATGCAGAAGTGGTTCTGCTGGTACGATTTGATTCCTGTTGGAATGTTTAAATTACACTAAG
TGTACTACTTTATATAATCAATGAAATTGCTAGACATGTTTTAGCAGGACTTTTCTAGGAAAGACTTATG
TATAATTGCTTTTTAAAATGCAGTGCTTTACTTTAAACTAAGGGGAACTTTGCGGAGGTGAAAACCTTTG
CTGGGTTTTCTGTTCAATAAAGTTTTACTATGAATGACCCTGAAAAAAAAAAAAAAAAAAAAAA
Human HERPUD1 mRNA sequence - var9 (public gi: 285960)
(SEQ ID NO: 45)
CGTGAACGGTCGTTGCAGAGATTGCGGGCGGCTGAGACGCCGCCTGCCTGGCACCTAGGAGCGCAGCGGA
GCCCCGACACCGCCGCCCCCGCCATGGAGTCCGAGACCGAACCCGAGCCCGTCACGCTCCTGGTGAAGAG
CCCCAACCAGCGCCACCGCGACTTGGAGCTGAGTGGCGACCGCGGCTGGAGTGTGGGCCACCTCAAGGCC
CACCTGAGCCGCGTCTACCCCGAGCGTCCGCGTCCAGAGGACCAGAGGTTAATTTATTCTGGGAAGCTGT
TGTTGGATCACCAATGTCTCAGGGACTTGCTTCCAAAGCAGGAAAAACGGCATGTTTTGCATCTGGTGTG
CAATGTGAAGAGTCCTTCAAAAATGCCAGAAATCAACGCCAAGGTGGCTGAATCCACAGAGGAGCCTGCT
CGTTCTAATCGGGGACAGTATCCTGAGGATTCCTCAAGTGATGGTTTAAGGCAAAGGGAAGTTCTTCGGA
ACCTTTCTTCCCCTGGATGGGAAAACATCTCAAGGCCTGAAGCTGCCCAGCAGGCATTCCAAGGCCTGGG
TCCTGGTTTCTCCGGTTACACACCCTATGGGTGGCTTCAGCTTTCCTGGTTCCAGCAGATATATGCACGA
CAGTACTACATGCAATATTTAGCAGCCACTGCTGCATCAGGCGCTTTTGTTCCACCACCAAGTGCACAAG
AGATACCTGTGGTCTCTGCACCTGCTCCAGCCCCTATTCACAACCAGTTTCCAGCTCAAAACCAGCCTGC
CAATCAGAATGCTGCTCCTCAAGTGGTTGTTAATCCTGGAGCCAATCAAAATTTGCCGATGAATGCACAA
GGTGGCCCTATTGTGGAAGAAGATGATGAAATAAATCGAGATTGGTTGGATTGGACCTATTCAGCAGCTA
CATTTTCTGTTTTTCTCAGTATCCTCTACTTCTACTCCTCCCTGAGCAGATTCCTCATGGTCATGGGGGC
CACCGTTGTTATGTACCTGCATCACGTTGGGTGGTTTCCATTTAGACCGAGGCCGGTTCAGAACTTCCCA
AATGATGGTCCTCCTCCTGACGTTGTAAATCAGGACCCCAACAATAACTTACAGGAAGGCACTGATCCTG
AAACTGAAGACCCCAACCACCTCCCTCCAGACAGGGATGTACTAGATGGCGAGCAGACCAGCCCCTCCTT
TATGAGCACAGCATGGCTTGTCTTCAAGACTTTCTTTGCCTCTCTTCTTCCAGAAGGCCCCCCAGCCATC
GCAAACTGATGGTGTTTGTGCTGTAGCTGTTGGAGGCTTTGACAGGAATGGACTGGATCACCTGACTCCA
GCTAGATTGCCTCTCCTGGACATGGCAATGATGAGTTTTTAAAAAACAGTGTGGATGATGATATGCTTTT
GTGAGCAAGCAAAAGCAGAAACGTGAAGCCGTGATACAAATTGGTGAACAAAAAATGCCCAAGGCTTCTC
ATGTGTTTATTCTGAAGAGCTTTAATATATACTCTATGTAGTTTAATAAGCACTGTACGTAGAAGGCCTT
AGGTGTTGCATGTCTATGCTTGAGGAACTTTTCCAAATGTGTGTGTCTGCATGTGTGTTTGTACATAGAA
GTCATAGATGCAGAAGTGGTTCTGCTGGTAAGATTTGATTCCTGTTGGAATGTTTAAATTACACTAAGTG
TACTACTTTATATAATCAATGAAATTGCTAGACATGTTTTAGCAGGACTTTTCTAGGAAAGACTTATGTA
TAATTGCTTTTTAAAATGCAGTGCTTTACTTTAAACTAAGGGGAACTTTGCGGAGGTGAAAACCTTTGCT
GGGTTTTCTGTTCAATAAAGTTTTACTATGAATGACCCTG
Human HERPUD1 mRNA sequence - var10 (public gi: 7661869)
(SEQ ID NO: 46)
GACGTGAACGGTCGTTGCAGAGATTGCGGGCGGCTGAGACGCCGCCTGCCTGGCACCTAGGAGCGCAGCG
GAGCCCCGACACCGCCGCCGCCGCCATGGAGTCCGAGACCGAACCCGAGCCCGTCACGCTCCTGGTGAAG
AGCCCCAACCAGCGCCACCGCGACTTGGAGCTGAGTCGCGACCGCGGCTGGAGTGTGGGCCACCTCAAGG
CCCACCTGAGCCGCGTCTACCCCGAGCGTCCGCGTCCAGAGGACCAGAGGTTAATTTATTCTGGGAAGCT
GTTGTTGGATCACCAATGTCTCAGGGACTTGCTTCCAAAGCAGGAAAAACGGCATGTTTTGCATCTGGTG
TGCAATGTGAAGAGTCCTTCAAAAATGCCAGAAATCAACGCCAAGGTGGCTGAATCCACAGAGGAGCCTG
CTGGTTCTAATCGGGGACAGTATCCTGAGGATTCCTCAAGTGATGGTTTAAGGCAAAGGGAAGTTCTTCG
GAACCTTTCTTCCCCTGGATGGGAAAACATCTCAAGGCCTGAAGCTGCCCAGCAGGCATTCCAAGGCCTG
GGTCCTGGTTTCTCCGGTTACACACCCTATGGGTGGCTTCAGCTTTCCTGGTTCCAGCAGATATATGCAC
GACAGTACTACATGCAATATTTAGCAGCCACTGCTGCATCAGGGGCTTTTGTTCCACCACCAAGTGCACA
AGAGATACCTGTGGTCTCTGCACCTGCTCCAGCCCCTATTCACAACCAGTTTCCAGCTGAAAACCAGCCT
GCCAATCAGAATGCTGCTCCTCAAGTGGTTGTTAATCCTGGAGCCAATCAAAATTTGCCGATGAATGCAC
AAGGTGGCCCTATTGTGGAAGAAGATGATGAAATAAATCGAGATTGGTTGGATTGGACCTATTCAGCAGC
TACATTTTCTGTTTTTCTCAGTATCCTCTACTTCTACTCCTCCCTGAGCAGATTCCTCATGGTCATGGGG
GCCACCGTTGTTATGTACCTGCATCACGTTGGGTGGTTTCCATTTAGACCGAGGCCGGTTCAGAACTTCC
CAAATGATGGTCCTCCTCCTGACGTTGTAAATCAGGACCCCAACAATAACTTACAGGAAGGCACTGATCC
TGAAACTGAAGACCCCAACCACCTCCCTCCAGACAGGGATGTACTAGATGGCGAGCAGACCAGCCCCTCC
TTTATGAGCACAGCATGGCTTGTCTTCAAGACTTTCTTTGCCTCTCTTCTTCCAGAAGGCCCCCCAGCCA
TCGCAAACTGATGGTGTTTGTGCTGTAGCTGTTGGAGGCTTTGACAGGAATGGACTGGATCACCTGACTC
CAGCTAGATTGCCTCTCCTGGACATGGCAATGATGAGTTTTTAAAAAACAGTGTGGATGATGATATGCTT
TTGTGAGCAAGCAAAAGCAGAAACGTGAAGCCGTGATACAAATTGGTGAACAAAAAATGCCCAAGGCTTC
TCATGTCTTTATTCTGAAGAGCTTTAATATATACTCTATGTAGTTTAATAAGCACTGTACGTAGAAGGCC
TTAGGTGTTGCATGTCTATGCTTGAGGAACTTTTCCAAATGTGTGTGTCTGCATGTGTGTTTGTACATAG
AAGTCATAGATGCAGAAGTGGTTCTGCTGGTACGATTTGATTCCTGTTGGAATGTTTAAATTACACTAAG
TGTACTACTTTATATAATCAATGAAATTGCTAGACATGTTTTAGCAGGACTTTTCTAGGAAAGACTTATG
TATAATTGCTTTTTAAAATGCAGTGCTTTACTTTAAACTAAGGGGAACTTTGCGGAGGTGAAAACCTTTG
CTGGGTTTTCTGTTCAATAAAGTTTTACTATGAATGACCCTGAAAAAAAAAAAAAAAAAAAAAA
Human HERPUD1 Protein sequence - var1 (public gi: 16507802)
(SEQ ID NO: 47)
MESETEPEPVTLLVKSPNQRHRDLELSGDRGWSVGHLKAHLSRVYPERPRPEDQRLIYSGKLLLDHQCLR
DLLPKEKRHVLHLVCNVKSPSKMPEINAKVAESTEEPAGSNRGQYPEDSSSDGLRQREVLRNLSSPGWEN
ISRHHVGWFPFRPRPVQNFPNDGPPPDVVNQDPNNNLQEGTDPETEDPNHLPPDRDVLDGEQTSPSEMST
AWLVFKTFFASLLPEGPPAIAN
Human HERPUD1 Protein sequence - var2 (public gi: 10441911)
(SEQ ID NO: 48)
MQYLAATAASGAFVPPPSAQEIPVVSAPAPAPIHNQFPAENQPANQNAAPQVVVNPGANQNLRMNAQGGP
IVEEDDEINRDWLDWTYSAATFSVFLSILYFYSSLSRFLMVMGATVVMYLHHVGWFPFRPRPVQNFPNDG
PPPDVVNQDPNNNLQEGTDPETEDPNHLPPDRDVLDGEQTSPSFMSTAWLVFKTFFASLLPEGPPAIAN
Human HERPUD1 Protein sequence - var3 (public gi: 3005723)
(SEQ ID NO: 49)
GHLKSHLSRVYPERPRPEDQRLIYSGKLLLDHQCLRDLLPKEKRHVLHLVCNVKSPSKMPEINAKVAEST
EEPAGSNRGQYPEDSSSDGLRQREVLRNLSSPGWENISRPEAAQQAFQGLGPGFSGYTPYGWLQLSWFQQ
IYARQYYMQYLAATAASGAFVPPPSAQEIPVVSAPAPAPIHNQFPAENQPANQNAAPQVVVNPGANQNLR
MNAQCGPIVEEDDEINRDWLDWTYSAATFSVFLSILYFYSSLSRFLMVMGATVVMYLHHVGWFPFRPRPV
QNFPNDGPPPDVVNQDPNNNLQEGTDPETEDPNHLPPDRDVLDGEQTSPSFMSTAWLVFKTFFASLLPEG
PPAIAN
Human HERPUD1 Protein sequence - var4 (public gi: 7661870)
(SEQ ID NO: 50)
MESETEPEPVTLLVKSPNQRHRDLELSGDRGWSVGHLKAHLSRVYPERPRPEDQRLIYSGKLLLDHQCLR
DLLPKQEKRHVLHLVCNVKSPSKMPEINAKVAESTEEPAGSNRGQYPEDSSSDGLRQREVLRNLSSPGWE
NISRPEAAQQAFQGLGPGFSGYTPYGWLQLSWFQQIYARQYYMQYLAATAASGAFVPPPSAQEIPVVSAP
APAPIHNQFPAENQPANQNAAPQVVVNPGANQNLRMNAQGGPIVEEDDEINRDWLDWTYSAATFSVFLSI
LYFYSSLSRFLMVMGATVVMYLHHVGWFPFRPRPVQNFPNDGPPPDVVNQDPNNNLQEGTDPETEDPNHL
PPDRDVLDGEQTSPSFMSTAWLVFKTFFASLLPEGPPAIAN
Rat HERPUD1 mRNA sequence (public gi: 16758961)
AAGACACCAAGTGTCGTTGTCGGGTCGCAGACGGCTGCGTCGCCGCCCGTTCGGCATCCCTGAGCGCAGT
CGAGCCTCCAGCGCCGCAGACATGGAGCCCGAGCCACAGCCCGAGCCGGTCACGCTGCTGGTGAAGAGCC
CCAATCAGCGCCACCGCGACTTGGAGCTGAGTGGCGACCGCGGTTGGAGTGTGAGTCGCCTCAAGGCCCA
CCTGAGCCGAGTCTACCCCGAACGCCCGCGCCCAGACGACCAGAGGTTAATTTATTCTGGGAAGCTGCTG
TTGGATCACCAATGTCTCCAAGACTTGCTTCCAAAGCAGGAAAAGCGACATGTTTTGCACCTCGTGTGCA
ATGTGAGGAGTCCCTCAAAAAAGCCAGAAGCCAGCACAAAGGGTGCTGAGTCCACAGAGCAGCCGGACAA
CACTAGTCAGGCACAGTATCCTGGGGATTCCTCAAGCGATGGCTTACGGGAAAGGGAAGTCCTTCGGAAC
CTTCCTCCCTCTGGATGGGAGAACGTCTCTAGGCCTGAAGCCGTCCAGCAGACTTTCCAAGGCCTCGGGC
CCGGCTTCTCTGGCTACACCACCTACGGGTGGCTGCAGCTCTCCTGGTTCCAGCAGATCTATGCAAGACA
GTACTACATGCAATACTTGGCTGCCACTGCTGCTTCAGGAGCTTTTGGCCCTACACCAAGTGCACAAGAA
ATACCTGTGGTCTCTACACCGGCTCCCGCCCCTATACACAACCAGTTTCCGGCAGAAAACCAGCCGGCCA
ATCAGAATGCAGCCGCTCAAGCGGTTGTTAATCCCGGAGCCAATCAGAACTTGCGGATGAATGCACAAGG
CGGCCCTCTGGTGGAAGAAGATGATGAGATAAACCGAGACTGGTTGGATTGGACCTACTCAGCAGCGACA
TTTTCCGTTTTCCTCAGCATTCTTTACTTCTACTCCTCCCTGAGCAGATTCCTCATGGTCATGGGCGCCA
CCGTAGTCATGTACCTGCACCACGTCGGGTGGTTTCCATTCAGACAGAGGCCAGTTCAGAACTTCCCAGA
TGACGGTCCCCCTCAGGAAGCTGCCAACCAGGACCCCAACAATAACCTCCAGGGAGGTTTGGACCCTGAA
ATGGAAGACCCCAACCGCCTCCCCGTAGGCCGTGAAGTGCTGGACCCTGAGCATACCAGCCCCTCGTTCA
TGAGCACAGCATGGCTAGTCTTCAAGACTTTCTTTGCCTCTCTTCTTCCGGAAGGCCCACCAGCCCTAGC
AAACTGATGGCCCCTGTGCTCTGTTGCTGGAGGCTTTCACAGCTTGGACTGGATCGTCCCCTGGCGTGGA
CTCGAGAGAGTCATTGAAAACCCACAGGATGACGATGTGCTTCTGTGCCAAGCAAAAGCACAAACTAAGA
CATGAAGCCGTGGTACAAACTGAACAGGGCCCCTCATGTCGTTATTCTGAAGAGCTTTAATGTATACTGT
ATGTAGTCTCATAGGCACTGTAAACAGAAGGCCCAGGGTCGCATGTTCTGCCTGAGCACCTCCCCAGACG
TGTGTGCATGTGTGCCGTACATGGAAGTCATAGACGTGTGTGCATGTGTGCTCTACATGGAAGTCATAGA
TGCAGAAACGGTTCTGCTGGTTCGATTTGATTCCTGTTGGAATGTTGCAATTACACTAAGTGTACTACTT
TATATAATCAGTGACTTGCTAGACATGTTAGCAGGACTTTTCTAGGAGAGACTTATTGTATCATTGCTTT
TTAAAACGCAGTGCTTACTTACTGAGGGCGGCGACTTGGCACAGGTAAAGCCTTTGCCGGGTTTTCTGTT
CAATAAGTTTTGCTATGAACGACAAAAAAAAAAAAA
Rat HERPUD1 Protein sequence (public gi: 16758962)
MEPEPQPEPVTLLVKSPNQRHRDLELSGDRGWSVSRLKAHLSRVYPERPRPEDQRLIYSGKLLLDHQCLQ
DLLPKQEKRHVLHLVCNVRSPSKKPEASTKGAESTEQPDNTSQAQYPGDSSSDGLREREVLRNLPPSGWE
NVSRPEAVQQTFQGLGPGFSGYTTYGWLQLSWFQQIYARQYYMQYLAATAASGAFGPTPSAQEIPVVSTP
APAPIHNQFPAENQPANQNAAAQAVVNPGANQNLRMNAQGGPLVEEDDEINRDWLDWTYSAATFSVFLSI
LYFYSSLSRFLMVMGATVVMYLHHVGWFPFRQRPVQNFPDDGPPQEAANQDPNNNLQGGLDPEMEDPNRL
PVGREVLDPEHTSPSPMSTAWLVFKTFFASLLPEGPPALAN
Mouse HERPUD1 mRNA sequence (public gi: 11612514)
AAAGACGCCAAGTGTCGTTGTGTGGTCTCAGACGGCTGCGTCGCCGCCCGTTCGGCATCCCTGAGCGCAG
TCGAGCCGCCAGCGACGCAGACATGCAGCCCGAGCCACAGCCCGAGCCGGTCACGCTGCTGGTGAAGAGT
CCCAATCAGCGCCACCGCGACTTGGAGCTGAGTGGCGACCGCAGTTGGAGTGTGAGTCGCCTCAACGCCC
ACCTGAGCCGAGTCTACCCCGAGCGCCCGCGTCCAGAGGACCAGAGGTTAATTTATTCTGGGAAGCTGCT
GTTGGATCACCAGTGTCTCCAAGATTTGCTTCCAAAGCAGGAAAAGCGACATGTTTTGCACCTTGTGTGC
AATGTGAAGAATCCCTCCAAAATGCCAGAAACCAGCACAAAGGGTGCTGAATCCACAGAGCAGCCGGACA
ACTCTAATCAGACACAGCATCCTGGGGACTCCTCAAGTGATGGTTTACGGCAAAGAGAAGTTCTTCGGAA
CCTTTCTCCCTCCGGATGGGAGAACATCTCTAGGCCTGAGGCTGTCCAGCAGACTTTCCAAGGCCTGGGG
CCTGGCTTCTCTGGCTACACAACGTATGGGTGGCTCCAGCTCTCCTGGTTCCAGCAGATCTATGCAAGGC
AGTACTACATGCAATACTTAGCTGCCACTGCTGCATCAGGAACTTTTGTCCCGACACCAAGTGCACAAGA
GATACCTGTGGTCTCTACACCTGCTCCGGCTCCTATACACAACCAGTTTCCGGCAGAAAACCAGCCGGCC
AATCAGAATGCAGCTGCTCAAGCGGTTGTCAATCCCGGAGCCAATCAGAACTTGCGGATGAATGCACAAG
GTGGCCCCCTGGTGGAGGAAGATGATGAGATAAACCGAGACTGGTTGGATTGGACCTATTCCGCAGCGAC
GTTTTCTGTTTTCCTCAGCATCCTTTACTTCTACTCCTCGCTGAGCAGATTTCTCATGGTCATGGGTGCC
ACTGTAGTCATGTACCTGCACCACGTCGGGTGGTTTCCGTTCAGACAGAGGCCAGTTCAGAACTTCCCGG
ATGATGGTGGTCCTCGAGATGCTGCCAACCAGGACCCCAACAATAACCTCCAGGGAGGTATGGACCCAGA
AATGGAAGACCCCAACCGCCTCCCCCCAGACCGCGAAGTGCTGGACCCTGAGCACACCAGCCCCTCGTTT
ATGAGCACAGCATGGCTAGTCTTCAAGACTTTCTTTGCCTCTCTTCTTCCAGAAGGCCCACCAGCCCTAG
CCAACTGATGGCCCTTGTGCTCTGTCGCTGGTGGCTTTGACAGCTCGGACTGGATCGTCTGGCTCCGGCT
CCTTTTCCTCCCCTGGCGTGGACTCGACAGAGTCATTGAAAACCCACAGGATGACATGTGCTTCTGTGCC
AAGCAAAAGCACAAACTAAGACATGAAGCCGTGGTACAAACTGAACAGGGCCCCTCATGTCGTTATTCTG
AAGAGCTTTAATGTATACTGTATGTAGTTTCATAGGCACTGTAAGCAGAAGGCCCAGGGTCGCATGTTCT
GCCTGAGCACCTCCCCAGATGTGTGTGCATGTGTGCTGTACATGGAAGTCATAGACGTGTGTGCATGTGT
GCTCTACATGGAAGTCATAGATGCAGAAACGGTTCTGCTGGTTCGATTTGATTCCTGTTGGAATGTTCAA
ATTACACTAAGTGTACTACTTTATATAATCAGTGAATTGCTAGACATGTTAGCAGGACTTTTCTAGGAGA
GACTTATGTATAATTGCTTTTTAAAATGCAGTGCTTTCCTTTAAACCGAGGGTGGCGACTTGGCAGAGGT
AAAACCTTTGCCGAGTTTTCTGTTCAATAAAGTTTTGCTATGAATGACTGT
Mouse HERPUD1 Protein sequence (public gi: 11612515)
MEPEPQPEPVTLLVKSPNQRHRDLELSGDRSWSVSRLKAHLSRVYPERPRPEDQRLIYSGKLLLDHQCLQ
DLLPKQEKRHVLHLVCNVKNPSKMPETSTKGAESTEQPDNSNQTQHPGDSSSDGLRQREVLRNLSPSGWE
NISRPEAVQQTFQGLGPGFSGYTTYGWLQLSWFQQIYARQYYMQYLAATAASGTFVPTPSAQEIPVVSTP
APAPIHNQFPAENQPANQNAAAQAVVNPGANQNLRMNAQGGPLVEEDDEINRDWLDWTYSAATFSVFLSI
LYFYSSLSRFLMVMGATVVMYLHHVGWFPFRQRPVQNFPDDGGPRDAANQDPNNNLQGGMDPEMEDPNRL
PPDREVLDPEHTSPSFMSTAWLVFKTFFASLLPEGPPALAN

### Example 12

### HERPUD1 Depletion by siRNA Reduces HIV Maturation

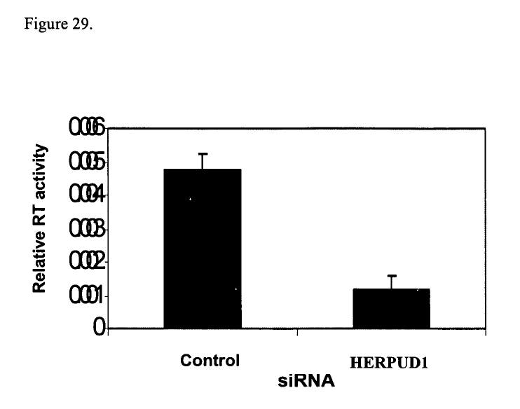

Hela SS6 cells were transfeted with siRNA directed against HERPUD1 and with a plsmid encoding HIV proviral genome (pNLenv-1). Twenty four hours post-HIV transfection, virus-like particles (VLP) secreted into the medium were isolated and reverse transcriptase activity was determined. HIV release of active RT is an indication for a release of processed and mature virus. When the levels of HERPUD1 were reduced RT activity was inhibited by 80%, demonstrating the importance of HERPUD1 in HIV-maturation. See FIG. 29.

Experimental Outline

Cell Culture and Transfection:

HeLa SS6 were kindly provided by Dr. Thomas Tuschl (the laboratory of RNA Molecular Biology, Rockefeller University, New York, N.Y.). Cells were grown in Dulbecco's modified Eagle's medium (DMEM) supplemented with 10% heat-inactivated fetal calf serum and 100 U/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) (HERPUD1: 5′-GGGAAGUUCUUCGGAACCUdTdT-3′ and 5′-dTdTCCCUUCAAGAAGCCUUGGA-5′) using lipofectamin 2000 (Invitrogen, Paisley, UK). A day following the initial transfection cells were split 1:3 in complete medium and co-transfected 24 hours later with HIV-1NLenv1 (2 μg per 6-well) (Schubert et al., J. Virol. 72:2280-88 (1998)) and a second portion of double-stranded siRNA.

Assay for Virus Release

Virus and virus-like particle (VLP) release was determined one day after transfection with the proviral DNA as previously described (Adachi et al., J. Virol. 59: 284-91 (1986); Fukumori et al., Vpr. Microbes Infect. 2: 1011-17 (2000); Lenardo et al., J. Virol. 76: 5082-93 (2002)). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in SDS-PAGE sample buffer. The corresponding cells were washed three times with phosphate-buffered saline (PBS) and then solubilized by incubation on ice for 15 minutes in lysis buffer containing the following components: 50 mM HEPES-NaOH, (pH 7.5), 150 mM NaCl, 1.5 mM MgCl₂, 0.5% NP-40, 0.5% sodium deoxycholate, 1 mM EDTA, 1 mM EGTA and 1:200 dilution of protease inhibitor cocktail (Calbiochem, La Jolla, Calif.). The cell detergent extract was then centrifuged for 15 minutes at 14,000×g at 4° C. The VLP sample and a sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected either after incubation with a secondary anti-rabbit horseradish peroxidase-conjugated antibody and detected by Enhanced Chemi-Luminescence (ECL) (Amersham Pharmacia) or after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry and the amount of released VLP was then determined by calculating the ratio between VLP-associated CA and intracellular CA and Pr55 as previously described (Schubert et al., J. Virol. 72:2280-88 (1998)).

Analysis of Reverse Transcriptase Activity in Supernatants

RT activity was determined in pelleted VLP (see above) by using an RT assay kit (Roche, Germany; Cat.No. 1468120). Briefly, VLP pellets were resuspended in 40 μl RT assay lysis buffer and incubated at room temperature for 30 minutes. At the end of incubation 20 μl RT assay reaction mix was added to each sample and incubation continued at 37° C. overnight. Samples (60 μl) were than transferred to MTP strip wells and incubated at 37° C. for 1 hour. Wells were washed five times with wash buffer and DIG-POD added for a one-hour incubation at 37° C. At the end of incubation wells were washed five times with wash buffer and ABST substrate solution was added and incubated until color developed. The absorbance was read in an ELISA reader at 405 nm (reference wavelength 492 nm). The resulting signal intensity is directly proportional to RT activity; RT concentration was determined by plotting against a known amount of RT enzyme included in separate wells of the reaction.

### Example 13

### POSH-Depleted Cells Have Lower Levels of Herp and it is not Monoubiguitinated

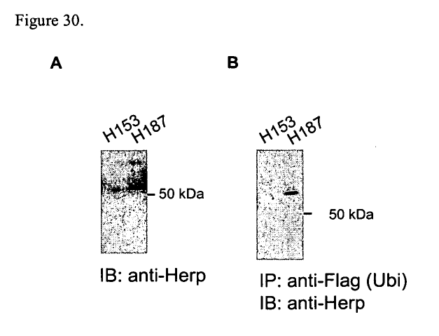

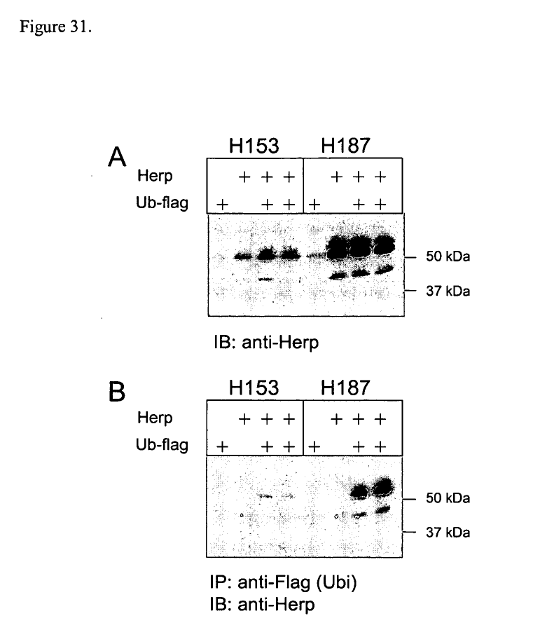

POSH-depleted cells and their control counterparts were lysed and immunoblotted with anti-herp antibodies. Cells depleted of POSH (H153 RNAi stables cell lines) cells have lower levels of Herp compared with control cells (H187 RNAi) (FIG. 30 panel A). When cells were transfected with a plasmid encoding flagged-tagged ubiquitin, and immunoprecipitated with anti-flag antibodies to immunoprecipitate ubiquitinated proteins, Herp was ubiquitinated only in H187 cells and not in H153 cells (FIG. 30 panel B). When the aforementioned cells were transfected with Herp-encoding plasmid, exogenous herp levels were also reduced in H153 cells compared to H187 cells (FIG. 31 panel A) and the ubiquitination of exogenous herp was reduced in the former cells, similar to endogenous Herp. The molecular weight of ubiquitinated Herp is as predicated to full-length Herp and does not seem as a high molecular weight smear, a characteristic of polyubiquitinated proteins. Thus POSH is responsible for the mono-ubiquitination of Herp, and in the absence of this modification herp is subjected to degradation, which may be mediated by the proteosome.

Materials and Methods

Plasmid generation
Full-length Herp was cloned from image clone MGC:45131 IMAGE:5575914 (GeneBank Accesion BC032673) into pCMV-SPORT6.
   (i)
   (ii) Antibody production
      Herp1 (amino acids 1 to 251) was amplified from a plasmid (3Gd4) obtained by yeast two hybrid screen for interactors of POSH. The amplified open reading frame was cloned into pGEX-6P, expressed in E. coli BL21 by induction with 1 mM IPTG and purified on glutathione-agarose. The purified protein was cleaved with Precision™ protease (Amersham Biosciences) and the GST moiety removed by glutathione chromatography. The protein was injected into rabbits (Washington Biotechnology) to produce anti-Herp1 sera.

(b) Transfections and antibody detection

Twenty-four hours prior to transfection POSH-RNAi clones (H153) or control-RNAi clones (H187) cells were plated in 10 cm dishes in growth medium (DMEM containing 10% fetal calf serum without antibiotics). Cells were transfected with lipofectamin 2000 (Invitrogen Corporation) and either Herp-expression plasmid (2.5 μg) or empty vector (2.5 μg) and a vector encoding Flag-tagged ubiquitin (1 μg). Twenty-four hours post-trasnfection cells were lysed in lysis buffer (50 mM Tris-HCl, pH7.6, 1.5 mM MgCl2, 150 mM NaCL, 10% glycerol, 1 mM EDTA, 1 mM EGTA, 0.5% NP-40 and 0.5% sodium deoxycholate, containing protease inhibitors) and subjected to immunoprecipitation with anti-Flag antibodies (Sigma, F7425) to precipitate ubiquitinated proteins. Immunoprecipitated material and total cell lysates were separated on 10% SDS-PAGE and transferred to nitrocellulose membranes which were immunoblotted with anti-Herp antibodies.

Generation of H187 and H153 Cell Lines

To relieve the necessity for multiple transfections and to improve the reproducibility of HPOSH reduction, we have generated two cell lines, H187 and H153 constitutively expressing an integrated control and hPOSH siRNA (respectively).

Construction of shRNA retroviral vectors—hPOSH scrambled oligonucleotide (5′-CACACACTGCCGTCAACTGTTCAAGAGACAGTTGACGGCAGTGTGTGTTT TTT-3′; and 5′-AATTAAAAAACACACACTGCCGTCAACTGTCTCTTGAACA GTTGACGGCAGTGTGTGGGCC-3′) were annealed and cloned into the ApaI-EcoRI digested pSilencer 1.0-U6 (Ambion, Inc.) to generate pSIL-scrambled. Subsequently, the U6-promoter and RNAi sequences were digested with BamHI, and blunted by end filling. The insert was cloned into the OliI site in the retroviral vector, pMSCVhyg (BD Biosciences Clontech), generating pMSCVhyg-U6-scrambled. The hPOSH oligonucleotide encoding RNAi against hPOSH (5′-AACAGAGGCCTTGGAAACCTGGAAGCTTGCAGGTTTCCAAGGCCTCT GTT-3′; and 5′-GATCAACAGAGGCCTTGGAAACCTGCAAGCTTCCAGGTTTCCAAGGC CTCTGTT-3′) were annealed and cloned into the BamHI-EcoRV site of pLIT-U6, generating pLIT-U6 hPOSH-230. The pLIT-U6 is an shRNA vector containing the human U6 promoter (amplified by PCR from human genomic DNA with the primers, 5′-GGCCCACTAGTCAAGGTCGGGCAGGAAGA-3′ and 5′-GCCGAATTCAAAAAGGATCCGGCGATATCCGGTGTTTCGTCCTTTCCA-3′) cloned into pLITMUS38 (New England Biolabs, Inc.) digested with SpeI-EcoRI. Subsequently, the U6 promoter-hPOSH shRNA (pLIT-U6 hPOSH-230 digested with SnaBI and PvuI) was cloned into the Oli1 site of pMSCVhyg (BD Biosciences Clontech) generating pMSCVhyg U6-hPOSH-230.

Recombinant retrovirus production—HEK 293T cells were transfected with retroviral RNAi plasmids (pMSCVhyg-U6-POSH-230 and pMSCVhyg-U6-scrambled and with plasmids encoding VSV-G and Moloney Gag-pol. Two days post-transfection, the retrovirus-containing medium was collected and filtered.

Infection and selection—Polybrene (Hexadimethrine bromide) (Sigma) (8 μg/ml) was added to the filtered and the treated medium was subsequently used to infect HeLa SS6 cells. Forty-eight hours post-infection clones were selected for RNAi expression by the addition of hygromycin (300 μg/ml). Clones expressing the scrambled and the hPOSH RNAi were termed H187 and H153 (respectively).

### Example 14

### HERPUD1 Associates with HIV-1 Nef

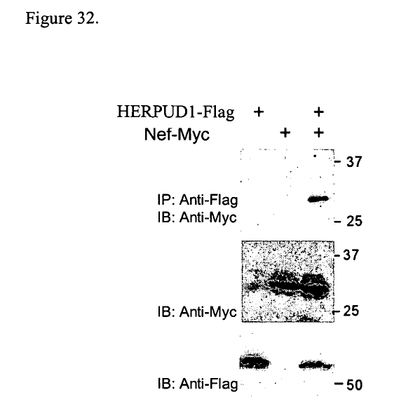

Hela SS6 cells were transfected with plasmids encoding HERPUD1-Flag or Nef-Myc. Twenty-four hours post transfection cells were lysed and subjected to immunoprecipitation with anti-Flag antibodies. Cell lysates and immunoprecipitated material were separated by SDS-PAGE and immunoblotted with anti-Flag and anti-Myc antibodies (as indicated) to detect HERPUD1 or Nef, respectively. (See FIG. 32).

Nef protein from pNLEnvl vector (SEQ ID NO: 51):

MGGKWSKSSVIGWPAVRERMRRAEPAADGVGAVSRDLEKHGAITSSNTAA
NNAACAWLEAQEEEEVGFPVTPQVPLRPMTYKAAVDLSHFLKEKGGLEGLI
HSQRRQDILDLWIYHTQGYFPDWQNYTPGPGVRYPLTFGWCYKLVPVEPD
KVEEANKGENTSLLHPVSLHGMDDPEREVLEWRFDSRLAFHHVARELHPEY
FKNC

### Example 15

### HERP Depletion Reduces Nef Protein Level

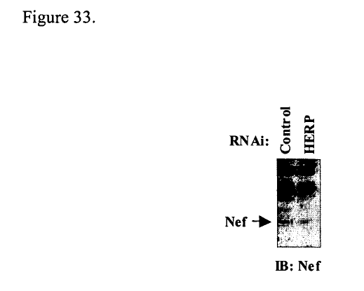

Hela SS6 cells were transfeted with siRNA directed against HERP and with a plsmid encoding HIV proviral genome (pNLenv-1). Twenty four hours post-HIV transfection, Nef protein level was determined by immunoblot with anti-Nef specific antibodies (CisBio). (See FIG. 33). The siRNA at 100 nM (HERP: 5′-GGGAAGUUCUUCGGAACCUdTdT-3′ (SEQ ID NO: 52) and 5′-dTdTCCCUUCAAGAAGCCUUGGA-5′(SEQ ID NO: 53)) transfected using lipofectamin 2000 (Invitrogen, Paisley, UK).

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject applications have been discussed, the above specification is illustrative and not restrictive. Many variations of the applications will become apparent to those skilled in the art upon review of this specification and the claims below. The full scope of the applications should be determined by reference to the claims, along with their full scope of equivalents, and the specification, along with such variations.

1. A method for identifying an agent that modulates an activity of a HERPUD1 polypeptide, the method comprising identifying an agent that modulates a complex comprising a HERPUD1 polypeptide and a Nef polypeptide, wherein an agent that modulates a complex comprising a HERPUD1 polypeptide and a Nef polypeptide is an agent that modulates an activity of the HERPUD1 polypeptide.

2. The method of claim 1, further comprising evaluating the effect of the test agent on a function of a virus, wherein an agent that inhibits a pro-infective or pro-replicative function of a virus is an antiviral agent.

3. The method of claim 1, further comprising contacting a cell infected with HIV with the agent and measuring the effect of the agent on a Nef-mediated process selected from the group consisting of: down-regulation of CD4 receptors; down-regulation of surface MHC class I molecules; enhancement of infectivity of HIV; and T cell activation.

4. The method of claim 1, wherein the agent decreases the level of HERPUD1 polypeptide in a cell.

5. The method of claim 1, wherein the agent decreases the amount of Nef polypeptide in a cell.

6. A method of inhibiting a Nef-mediated process in a cell infected with HIV, comprising contacting the cell with a POSH antagonist, whereby a Nef-mediated process is inhibited in the cell.

7. The method of claim 6, wherein the POSH antagonist inhibits a POSH activity.

8. The method of claim 7, wherein the POSH antagonist inhibits the ubiquitin ligase activity of a POSH polypeptide.

10. The method of claim 7, wherein the POSH antagonist decreases the level of POSH polypeptide in the cell.

9. The method of claim 6, wherein the POSH antagonist inhibits one or more of the following Nef-mediated processes: down-regulation of CD4 receptors; down-regulation of surface MHC class I molecules; enhancement of infectivity of HIV; and T cell activation.

10. The method of claim 6, wherein contacting the cell with the POSH antagonist causes a decrease in the amount of Nef polypeptide in the cell.

11. The method of claim 6, wherein contacting the cell with the POSH antagonist causes a decrease in the amount of Nef polypeptide that is membrane localized in the cell.

12. The method of claim 6, wherein the cell is situated in a subject that is infected with HIV and wherein contacting the cell with the POSH antagonist comprises administering the POSH antagonist to the subject.

13. The method of claim 6, wherein the cell is a cultured cell.

14. A method of inhibiting the progression of AIDS in a subject infected with HIV comprising administering to the subject a POSH antagonist, whereby the progression of AIDS is inhibited in the subject.

15. The method of claim 14, wherein the POSH antagonist inhibits a Nef-mediated process.

16. The method of claim 14, wherein the progression of AIDS is inhibited in the subject by inhibiting a decline in CD4⁺ T-cell counts.

17. The method of claim 14, wherein the POSH antagonist inhibits the ubiquitin ligase activity of a POSH polypeptide.

18. The method of claim 15, wherein the POSH antagonist inhibits one or more of the following Nef-mediated processes in an HIV infected cell of the subject: down-regulation of CD4 receptors; down-regulation of surface MHC class I molecules; enhancement of infectivity of HIV; and T cell activation.

19. The method of claim 15, wherein the POSH antagonist causes a decrease in the amount of Nef polypeptide in an HIV infected cell of the subject.

20. The method of claim 15, wherein the POSH antagonist causes a decrease in the amount of Nef polypeptide that is membrane localized in an HIV infected cell of the subject.
